SEQUENCE LISTING (58 sequences)

SEQ ID NO: 1 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 4 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 5 - protein, 485 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 263 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 7 - DNA, 789 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 8 - protein, 20 aa, Homo sapiens
MYRMQLLSCIALSLALVTNS

SEQ ID NO: 9 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 10 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 11 - protein, 69 aa, Unknown
  Description of Unknown CD8 sequence
STTTKPVLRTPSPVHPTGTSQPQRPEDCRPRGSVKGTGLDFACDIYIWAPLAGICVALLLSLIITLICY

SEQ ID NO: 12 - DNA, 207 bp, Unknown
  Description of Unknown CD8 sequence
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 13 - protein, 188 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQRRRNPQEGVYNALQKDKMAEAYSEIGTKGERRRGKGHDGLYQDSHFQAVQFGNRREREGSELTRTLGLRARPKACRHKKPLSLPAAVS

SEQ ID NO: 14 - protein, 113 aa, Unknown
  Description of Unknown CD3 zeta sequence
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 15 - DNA, 342 bp, Unknown
  Description of Unknown CD3 zeta sequence
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 16 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 17 - DNA, 123 bp, Mus musculus
aatagtagaaggaacagactccttcaaagtgactacatgaacatgactccccggaggcctgggctcactcgaaagccttaccagccctacgcccctgccagagactttgcagcgtaccgcccc

SEQ ID NO: 18 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 19 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 20 - DNA, 1455 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - protein, 178 aa, Mus musculus
MEICWGPYSHLISLLLILLFHSEAACRPSGKRPCKMQAFRIWDTNQKTFYLRNNQLIAGYLQGPNIKLEEKIDMVPIDLHSVFLGIHGGKLCLSCAKSGDDIKLQLEEVNITDLSKNKEEDKRFTFIRSEKGPTTSFESAACPGWFLCTTLEADRPVSLTNTPEEPLIVTKFYFQEDQ

SEQ ID NO: 22 - DNA, 81 bp, Homo sapiens
ttttgggtgctggtggtggttggtggagtcctggcttgctatagcttgctagtaacagtggcctttattattttctgggtg

SEQ ID NO: 23 - protein, 15 aa, Artificial Sequence
  Description of Artificial Sequence Synthetic peptide
GGGGSGGGGSGGGGS

SEQ ID NO: 24 - protein, 20 aa, Mus musculus
MYSMQLASCVTLTLVLLVNS

SEQ ID NO: 25 - protein, 20 aa, Homo sapiens
METPAQLLFLLLLWLPDTTG

SEQ ID NO: 26 - protein, 20 aa, Mus musculus
METDTLLLWVLLLWVPGSTG

SEQ ID NO: 27 - protein, 21 aa, Homo sapiens
MALPVTALLLPLALLLHAARP

SEQ ID NO: 28 - protein, 16 aa, Homo sapiens
MKWVTFISLLFSSAYS

SEQ ID NO: 29 - protein, 30 aa, Homo sapiens
MDSKGSSQKGSRLLLLLVVSNLLLCQGVVS

SEQ ID NO: 30 - protein, 41 aa, Homo sapiens
RSKRSRLLHSDYMNMTPRRPGPTRKHYQPYAPPRDFAAYRS

SEQ ID NO: 31 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 32 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - protein, 27 aa, Homo sapiens
FWVLVVVGGVLACYSLLVTVAFIIFWV

SEQ ID NO: 35 - DNA, 81 bp, Homo sapiens; identical to SEQ ID NO: 22

SEQ ID NO: 36 - protein, 108 aa, Artificial Sequence
  Description of Artificial Sequence Synthetic polypeptide
AIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 37 - DNA, 324 bp, Artificial Sequence
  Description of Artificial Sequence Synthetic polynucleotide
gcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 38 - RNA, 20 nt, Artificial Sequence
  Description of Artificial Sequence Synthetic oligonucleotide
ccaaacuucuccccgaucug

SEQ ID NO: 39 - RNA, 20 nt, Artificial Sequence
  Description of Artificial Sequence Synthetic oligonucleotide
uguguaucuucauagaaggu

SEQ ID NO: 40 - RNA, 20 nt, Artificial Sequence
  Description of Artificial Sequence Synthetic oligonucleotide
ucuucauagaagguuggaca

SEQ ID NO: 41 - RNA, 20 nt, Artificial Sequence
  Description of Artificial Sequence Synthetic oligonucleotide
caaaauagauagaagaugaa

SEQ ID NO: 42 - RNA, 20 nt, Artificial Sequence
  Description of Artificial Sequence Synthetic oligonucleotide
acgauacagagaugcaacac

SEQ ID NO: 43 - DNA, 21 bp, Artificial Sequence
  Description of Artificial Sequence Synthetic oligonucleotide
ggctatgcattcagtagctac

SEQ ID NO: 44 - DNA, 18 bp, Artificial Sequence
  Description of Artificial Sequence Synthetic oligonucleotide
tatcctggagatggtgat

SEQ ID NO: 45 - DNA, 39 bp, Artificial Sequence
  Description of Artificial Sequence Synthetic oligonucleotide
aagaccattagttcggtagtagatttctactttgactac

SEQ ID NO: 46 - DNA, 33 bp, Artificial Sequence
  Description of Artificial Sequence Synthetic oligonucleotide
aaggccagtcagaatgtgggtactaatgtagcc

SEQ ID NO: 47 - DNA, 21 bp, Artificial Sequence
  Description of Artificial Sequence Synthetic oligonucleotide
tcggcaacctaccggaacagt

SEQ ID NO: 48 - protein, 7 aa, Artificial Sequence
  Description of Artificial Sequence Synthetic peptide
GYAFSSY

SEQ ID NO: 49 - protein, 6 aa, Artificial Sequence
  Description of Artificial Sequence Synthetic peptide
YPGDGD

SEQ ID NO: 50 - protein, 13 aa, Artificial Sequence
  Description of Artificial Sequence Synthetic peptide
KTISSVVDFYFDY

SEQ ID NO: 51 - protein, 11 aa, Artificial Sequence
  Description of Artificial Sequence Synthetic peptide
KASQNVGTNVA

SEQ ID NO: 52 - protein, 7 aa, Artificial Sequence
  Description of Artificial Sequence Synthetic peptide
SATYRNS

SEQ ID NO: 53 - protein, 9 aa, Artificial Sequence
  Description of Artificial Sequence Synthetic peptide
QQYNRYPYT

SEQ ID NO: 54 - protein, 122 aa, Artificial Sequence
  Description of Artificial Sequence Synthetic polypeptide
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 55 - protein, 108 aa, Artificial Sequence
  Description of Artificial Sequence Synthetic polypeptide
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 56 - DNA, 27 bp, Artificial Sequence
  Description of Artificial Sequence Synthetic oligonucleotide
caacaatataacaggtatccgtacacg

SEQ ID NO: 57 - DNA, 366 bp, Artificial Sequence
  Description of Artificial Sequence Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctca

SEQ ID NO: 58 - DNA, 324 bp, Artificial Sequence
  Description of Artificial Sequence Synthetic polynucleotide
gacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

(19) United States
(12) Patent Application Publication
(10) Pub. No.: US 2020/0268793 A1
(43) Pub. Date: Aug. 27, 2020

(54) METHODS AND COMPOSITIONS FOR ALLEVIATING CYTOKINE RELEASE SYNDROME

(71) Applicant: MEMORIAL SLOAN-KETTERING CANCER CENTER, New York, NY (US)

(72) Inventors: Michel Sadelain, New York, NY (US); Theodoros Giavridis, New York, NY (US)

(73) Assignee: MEMORIAL SLOAN-KETTERING CANCER CENTER, New York, NY (US)

(21) Appl. No.: 15/931,027

(22) Filed: May 13, 2020

Related U.S. Application Data
(63) Continuation of application No. PCT/US2018/061795, filed on Nov. 19, 2018.
(60) Provisional application No. 62/587,965, filed on Nov. 17, 2017.

(51) Int. Cl. A61K 35/17; C07K 14/715; C07K 14/725; C07K 14/705; A01K 67/027

(52) U.S. Cl. CPC A61K 35/17; C07K 14/7155; C07K 14/7051; C07K 14/70575; A01K 2267/0331; C07K 2319/02; C07K 2319/03; A01K 2207/12; A01K 2227/105; A01K 67/0271

(57) ABSTRACT

The present disclosure provides methods and compositions for treating cancers and pathogens. It relates to an immunoresponsive cell comprising an antigen-recognizing receptor (e.g., a chimeric antigen receptor (CAR) or a T cell receptor (TCR)), and expressing a secretable IL-1Ra polypeptide.

### CROSS-REFERENCE TO RELATED APPLICATION

This application a Continuation of International Patent Application No. PCT/US18/61795 filed on Nov. 19, 2018, which claims priority to U.S. Provisional Application No. 62/587,965 filed on Nov. 17, 2017, the content of which is hereby incorporated by reference in its entirety, and to which priority is claimed.

### SEQUENCE LISTING

The specification further incorporates by reference the Sequence Listing submitted herewith via EFS on May 13, 2020. Pursuant to 37 C.F.R. § 1.52(e)(5), the Sequence Listing text file, identified as 0727341076_SL.txt, is 48,491 bytes and was created on May 13, 2020. The Sequence Listing electronically filed herewith, does not extend beyond the scope of the specification and thus does not contain new matter.

### INTRODUCTION

The presently disclosed subject matter provides methods and compositions for enhancing the immune response toward cancers and pathogens. It relates to immunoresponsive cells comprising antigen-recognizing receptors (e.g., chimeric antigen receptors (CARs) or T cell receptors (TCRs)) that are engineered to express an Interleukin-1 receptor antagonist (“IL-1Ra”) polypeptide. These engineered immunoresponsive cells are antigen-directed, promote recruitment of other cytokines and exhibit enhanced anti-target efficacy.

### BACKGROUND OF THE INVENTION

Chimeric Antigen Receptor (CAR) modified T cells have shown extraordinary promise in the clinic and are now an F.D.A. approved modality in relapse-refractory B cell Acute Lymphoblastic Leukemia (B-ALL) and Diffuse Large B cell Lymphoma (DLBCL). Despite its remarkable therapeutic benefit, CAR T cell therapy can induce toxicities, among which, Cytokine Release Syndrome (CRS) is a major concern. CRS is a commonly occurring and potentially lethal toxicity that typically presents itself within days after CAR T cell infusion. In its severe form, CRS can present symptoms such as fever, hypotension, respiratory failure and elevation of pro-inflammatory cytokines, including IL-6. Thus, CRS can be a hindrance to the broad application of CAR T cells¹⁻⁴. Therefore, there is a need for an effective treatment of CRS and/or a form of CART cell that reduces or avoids CRS.

Moreover, there are currently no reported mouse models in which current clinical CRS treatments can be validated and new treatment modalities tested. Therefore, there is a need for a suitable animal model for studying CRS.

### SUMMARY OF THE INVENTION

The presently disclosed subject matter provides immunoresponsive cells (e.g., T cells, Tumor Infiltrating Lymphocytes, Natural Killer (NK) cells, cytotoxic T lymphocytes (CTLs), Natural Killer T (NK-T) cells or regulatory T cells) that (a) express an antigen-recognizing receptor (e.g., CAR or TCR) directed toward a target antigen of interest, and (b) express (and secrete) an interleukin 1 receptor antagonist (“IL-1Ra”) polypeptide. In certain non-limiting embodiments, the immunoresponsive cell comprises a nucleic acid encoding an IL-1Ra polypeptide (e.g., IL-1Ra polypeptide-encoding nucleic acid), in expressible form.

In certain non-limiting embodiments, the presently disclosed subject matter provides an immunoresponsive cell (a) comprising an antigen-recognizing receptor that binds to an antigen, and (b) expressing or secreting an IL-1Ra polypeptide. In certain embodiments, the immunoresponsive cell comprises an exogenous IL-1Ra polypeptide. In certain embodiments, the immunoresponsive cell comprises a nucleic acid encoding an IL-1Ra polypeptide. In certain embodiments, binding of the antigen-recognizing receptor to the antigen is capable of activating the immunoresponsive cell. In certain embodiments, the antigen-recognizing receptor is a CAR.

In certain non-limiting embodiments, the presently disclosed subject matter provides an immunoresponsive cell comprising (a) an antigen-recognizing receptor (e.g., CAR or TCR) directed toward a target antigen of interest, and (b) a modified promoter at an endogenous (native) IL-1Ra gene locus. In certain embodiments, the modified promoter enhances the gene expression of the endogenous IL-1Ra gene locus. In certain non-limiting embodiments, the modification comprises replacement of an endogenous promoter with a constitutive promoter or an inducible promoter, or insertion of a constitutive promoter or inducible promoter to the promoter region of the endogenous IL-1Ra gene locus. In certain non-limiting embodiments, the constitutive promoter is selected from the group consisting of a CMV promoter, an EF1a promoter, a SV40 promoter, a PGK1 promoter, a Ubc promoter, a beta-actin promoter, and a CAG promoter. In certain non-limiting embodiments, the inducible promoter is selected from the group consisting of a tetracycline response element (TRE) promoter and an estrogen response element (ERE) promoter.

In certain embodiments, the immunoresponsive cell constitutively expresses the IL-1Ra polypeptide (mature or non-mature form of IL-1Ra protein). In certain embodiments, the IL-1Ra polypeptide is secreted. The antigen-recognizing receptor can be a TCR or a CAR. In certain embodiments, the antigen-recognizing receptor is a CAR. In certain embodiments, the immunoresponsive cell is selected from the group consisting of a T cell (e.g., a cytotoxic T lymphocyte (CTL), a regulatory T cell, or a Natural Killer T (NK-T) cell), a Natural Killer (NK) cell, a human embryonic stem cell, and a pluripotent stem cell from which lymphoid cells may be differentiated, a macrophage, a neutrophil, a monocyte, and a dendritic cell. In certain embodiments, the immunoresponsive cell is a T cell. In certain embodiments, the immunoresponsive cell is autologous or allogenic.

The presently disclosed subject matter further provides immunoresponsive cells comprising a modified CD40L. The modification can be selected from the group consisting of knock-down of CD40L, knock-out of CD40L, introduction of one or more mutation in a CD40L gene, modification of the endogenous promoter of a CD40L gene, modification of the endogenous enhancer elements of a CD40L gene, modification of the transcription factors that control CD40L expression, and combinations thereof.

The presently disclosed subject matter further provides methods for producing an immunoresponsive cell disclosed herein. In certain embodiments, the methods comprise introducing into an immunoresponsive cell (a) a first nucleic acid sequence that encodes an antigen-recognizing receptor that binds to an antigen, and (b) a second nucleic acid sequence that encodes an IL-1Ra polypeptide. In certain embodiments, the methods comprise introducing into an immunoresponsive cell (a) a first nucleic acid sequence that encodes an antigen-recognizing receptor that binds to an antigen, and (b) a second nucleic acid sequence that encodes a modified CD40L.

The presently disclosed subject matter further provides various nucleic acid compositions. In certain embodiments, the nucleic acid composition comprises (a) a first nucleic acid sequence encoding an antigen-recognizing receptor (e.g., a CAR or TCR) that binds to an antigen and (b) a second nucleic acid sequence encoding an IL-1Ra polypeptide (mature or non-mature form of IL-1Ra). In certain embodiments, the nucleic acid composition comprises (a) a first nucleic acid sequence encoding an antigen-recognizing receptor (e.g., a CAR or TCR) that binds to an antigen and (b) a second nucleic acid sequence encoding a modified CD40L.

In certain non-limiting embodiments, the first or the second nucleic acid sequence is operably linked to a promoter element constitutively or inducibly expressed in the immunoresponsive cell. The promoter for the first nucleic acid sequence may be the same or different from the promoter for the second nucleic acid sequence. In certain non-limiting embodiments, each of the first and second nucleic acid sequences is operably linked to a promoter element constitutively or inducibly expressed in the immunoresponsive cell. One or both of the first and second nucleic acid sequences may be comprised in a vector, which may be the same vector (bicistronic) or separate vectors. In certain non-limiting embodiments, the vector is a virus vector, e.g., a retroviral vector.

In certain embodiments, the nucleic acid composition is comprised in a vector. In certain non-limiting embodiments, the vector is a virus vector, e.g., a retroviral vector. The presently disclosed subject matter also provides a vector comprising the nucleic acid composition disclosed herein.

The presently disclosed subject matter also provides various methods of treatments. For example, the presently disclosed subject matter provides methods of treating and/or preventing a neoplasm in a subject, methods of reducing tumor burden in a subject, methods of lengthening survival of a subject having neoplasm (e.g., cancer), methods of reducing at least one symptom of cytokine release syndrome (CRS) in a subject, methods of reducing the level of a cytokine in a subject, methods of reducing the level of a chemokine in a subject, and methods of treating or alleviating CRS in a subject who receives an immunotherapy, and methods of treating blood cancer in a subject.

In certain embodiments, the level of a cytokine is reduced. In certain embodiments, the cytokine is a pro-inflammatory cytokine. In certain embodiments, the cytokine is selected from the group consisting of IL-1 alpha, IL-1 beta, IL-6, IL-8, IL-10, TNF-α, IFN-γ, IL-5, IL-2, IL-4, G-CSF, GM-CSF, M-CSF, IL-12, IL-15, and IL-17.

In certain embodiments, the chemokine is selected from the group consisting of CCL2, CCL3, CCL5, and CXCL1.

In certain non-limiting embodiments, the immunoresponsive cells reduce the level of one or more cytokine. In certain non-limiting embodiments, the one or more cytokine is selected from the group consisting of IL-1a, IL-1β, IL-6, IL-8, IL-10, TNF-α, IFN-γ, IL-5, IL-2, IL-4, G-CSF, GM-CSF, M-CSF, IL-12, IL-15, and IL-17. In certain non-limiting embodiments, the immunoresponsive cells reduce the level of one or more chemokine. In certain embodiments, the one or more chemokine is selected from the group consisting of CCL2, CCL3, CCL5, and CXCL1.

In certain embodiments, each of the various methods disclosed herein comprises administering to the subject an effective amount of the immunoresponsive cells or the pharmaceutical composition disclosed herein. In certain non-limiting embodiments, the method described herein does not comprise administering another therapy for preventing, treating and/or alleviating CRS.

In certain embodiments, each of the various methods disclosed herein comprises administering to the subject an antibody that binds to CD40L and an effective amount of the immunoresponsive cells, wherein the immunoresponsive cell comprises an antigen-recognizing receptor that binds to an antigen.

In certain embodiments, each of the various methods disclosed herein comprises administering to the subject an inhibitor of IL-1 signaling and an immunoresponsive cell comprising an antigen-recognizing receptor that binds to an antigen. In certain embodiments, the inhibitor of IL-1 signaling is selected from the group consisting of IL-1 blocking agents, IL-1R1 blocking agents, and combinations thereof. In certain embodiments, the IL-1 blocking agents are selected from the group consisting of IL-1Ra polypeptides, antibodies that bind to IL-1α, antibodies that bind to IL-1β, antibodies that bind to both IL-1α and IL-1β, and combinations thereof. In certain embodiments, the IL-1R1 blocking agents are selected from the group consisting of antibodies that bind to IL-1R1, antibodies that bind the IL-1 receptor accessory protein (IL-1RAP/IL-1RAcP), IL-1 receptor 2 (IL-1R2/IL-1RII) polypeptides, and combinations thereof. In certain embodiments, the IL-1Ra polypeptide is anakinra. In certain embodiments, the IL-1 blocking agent is rilonacept. In certain embodiments, the antibody that binds to IL-1β is canakinumab.

The presently disclosed subject matter provides uses of the immunoresponsive cell disclosed herein or the composition disclosed herein for use in a therapy, e.g., for use in reducing tumor burden, treating and/or preventing a neoplasm, lengthening survival of a subject having a neoplasm, and/or reducing at least one symptom of cytokine release syndrome (CRS) in response to a cancer or pathogen in a subject.

The presently disclosed subject matter provides uses of an antibody that binds to CD40L and an effective amounts of immunoresponsive cells, wherein the immunoresponsive cell comprises an antigen-recognizing receptor that binds to an antigen or the composition comprising thereof for use in a therapy, e.g., for use in reducing tumor burden, treating and/or preventing a neoplasm, lengthening survival of a subject having a neoplasm, and/or reducing at least one symptom of cytokine release syndrome (CRS) in response to a cancer or pathogen in a subject.

The presently disclosed subject matter provides uses of an inhibitor of IL-1 signaling and an immunoresponsive cells comprising an antigen-recognizing receptor that binds to an antigen or the composition comprising thereof for use in a therapy, e.g., for use in reducing tumor burden, treating and/or preventing a neoplasm, lengthening survival of a subject having a neoplasm, and/or reducing at least one symptom of cytokine release syndrome (CRS) in response to a cancer or pathogen in a subject.

The presently disclosed subject matter provides a kit for treating and/or preventing a neoplasm (e.g., cancer) or a pathogen infection, reducing tumor burden in a subject, lengthening survival of a subject having neoplasm (e.g., cancer), and/or treating or alleviating CRS in a subject who receives an immunotherapy. In certain embodiments, the kit comprises the immunoresponsive cells disclosed herein, the pharmaceutical composition disclosed herein, the nucleic acid composition disclosed herein, or the vector disclosed herein. In certain embodiments, the kit further comprises written instructions for treating and/or preventing a neoplasm or a pathogen infection, reducing tumor burden in a subject, lengthening survival of a subject having neoplasm (e.g., cancer), and/or treating or alleviating CRS in a subject who receives an immunotherapy.

In various non-limiting embodiments, the immunoresponsive cell is autologous or allogeneic to its intended recipient subject.

In various embodiments of any of the aspects delineated herein, the antigen-recognizing receptor is a TCR or a CAR. In various embodiments of any of the aspects delineated herein, the antigen-recognizing receptor is exogenous or endogenous. In various embodiments of any of the aspects delineated herein, the antigen-recognizing receptor is recombinantly expressed. In various embodiments of any of the aspects delineated herein, the antigen-recognizing receptor is expressed from a vector. In various embodiments of any of the aspects delineated herein, the antigen-recognizing receptor is a CAR. In certain embodiments, the CAR comprises an extracellular antigen-binding domain, a transmembrane domain, and an intracellular signaling domain. In certain embodiments, the CAR is 1928z.

In various embodiments of any of the aspects delineated herein, the antigen-recognizing receptor is a TCR. In certain embodiments, the TCR is a recombinant TCR. In certain embodiments, the TCR is a non-naturally occurring TCR. In certain embodiments, the TCR differs from any naturally occurring TCR by at least one amino acid residue. In certain embodiments, the TCR is modified from a naturally occurring TCR by at least one amino acid residue.

In various embodiments of any of the aspects delineated herein, the antigen to which the antigen-recognizing receptor binds is a tumor antigen or a pathogen antigen. In certain embodiments, the antigen is a tumor antigen. In various embodiments of any of the aspects delineated herein, the tumor antigen is selected from the group consisting of CD19, MUC16, MUC1, CA1X, CEA, CD8, CD7, CD10, CD20, CD22, CD30, CD33, CLL1 CD34, CD38, CD41, CD44, CD49f, CD56, CD74, CD133, CD138, a cytomegalovirus (CMV) infected cell antigen, EGP-2, EGP-40, EpCAM, erb-B2,3,4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, Mesothelin, ERBB2, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, ERBB, ITGB5, PTPRJ, SLC30A1, EMC10, SLC6A6, TNFRSF1B, CD82, ITGAX, CR1, DAGLB, SEMA4A, TLR2, LTB4R, P2RY13, LILRB2, EMB, CD96, LILRB3, LILRA6, LILRA2, ADGRE2, LILRB4, CD70, CCR1, CCR4, TACI, TRBC1, and TRBC2. In certain embodiments, the antigen is CD19. Amino acid sequences that specifically bind to said antigens are known in the art or may be prepared using methods known in the art; examples include immunoglobulins, variable regions of immunoglobulins (e.g. variable fragment (“Fv”) or bivalent variable fragment (“Fab”)), single chain antibodies, etc. In certain embodiments, the antigen is a pathogen antigen.

In various non-limiting embodiments of any of the aspects delineated herein, the exogenous IL-1Ra polypeptide is secreted. In various non-limiting embodiments of any of the aspects delineated herein, the IL-1Ra polypeptide is comprised in (and expressed from) a vector. In various non-limiting embodiments of any of the aspects delineated herein, the IL-1Ra polypeptide comprises a heterologous signal sequence at the amino-terminus (e.g., a signal sequence that is not naturally associated with IL-1Ra). In various embodiments of any of the aspects delineated herein, the heterologous signal sequence is selected from the group consisting of IL-2 signal sequence, the kappa leader sequence, the CD8 leader sequence, and combinations and/or synthetic variations thereof which retain the capacity to promote secretion of IL-1Ra polypeptide (either mature or non-mature). In certain embodiments, the IL-1Ra polypeptide is fused to a transmembrane polypeptide to obtain membrane-bound IL-1Ra on the immunoresponsive cells. In certain embodiments, the IL-1Ra peptide is a mature form of IL-1Ra protein, or a functional fragment thereof. In certain embodiments, the IL-1Ra peptide comprises an amino acid sequence that is at least about 80% homologous to the sequence set forth in SEQ ID NO: 4 or SEQ ID NO: 21. In certain embodiments, wherein the IL-1Ra peptide comprises the amino acid sequence set forth in SEQ ID NO: 4 or SEQ ID NO: 21. In various embodiments of any of the aspects delineated herein, the IL-1Ra polypeptide enhances an immune response of the immunoresponsive cell. In certain embodiments, the exogenous IL-1Ra polypeptide prevents or alleviates CRS. In certain embodiments, the exogenous IL-1Ra polypeptide reduces the production of one or more cytokine. In certain non-limiting embodiments, the one or more cytokine is selected from the group consisting of IL-1 alpha, IL-1 beta, IL-6, IL-8, IL-1β, TNF-α, IFN-γ, IL-5, IL-2, IL-4, G-CSF, GM-CSF, M-CSF, IL-12, IL-15, and IL-17. In certain embodiments, the exogenous IL-1Ra polypeptide reduces the production of one or more chemokine. In certain embodiments, the one or more chemokine is selected from the group consisting of CCL2, CCL3, CCL5, and CXCL1.

In various non-limiting embodiments of any of the aspects delineated herein, the immunoresponsive cell reduces and/or prevents the activation of an endogenous myeloid cell. In certain embodiments, the endogenous myeloid cell is selected from the group consisting of a monocyte, a macrophage, a neutrophil, a basophil, an eosinophil, an erythrocyte, a dendritic cell, a megakaryocyte, and immature myeloid cell of granulocytic or monocytic lineage. In certain embodiments, the endogenous myeloid cell is a macrophage.

In various embodiments of any of the aspects delineated herein, the method reduces the number of tumor cells, reduces tumor size, eradicates the tumor in the subject, reduces the tumor burden in the subject, eradicates the tumor burden in the subject, increases the period of time to relapse/recurrence, and/or increases the period of survival.

Illustrative neoplasia for which the presently disclosed subject matter can be used include, but are not limited to leukemias (e.g., acute leukemia, acute lymphocytic leukemia, acute myeloid leukemia (AML), acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In various non-limiting embodiments of any of the aspects delineated herein, the neoplasm is selected from the group consisting of blood cancer, B cell leukemia, multiple myeloma, acute lymphoblastic leukemia (ALL), acute myeloid leukemia (AML), chronic lymphocytic leukemia, non-Hodgkin's lymphoma, and ovarian cancer. In certain embodiments, the blood cancer is one or more of B cell leukemia, multiple myeloma, acute lymphoblastic leukemia (ALL), chronic lymphocytic leukemia, and non-Hodgkin's lymphoma. In certain embodiments, the antigen is CD19. In certain embodiments, the neoplasm is ovarian cancer, and the antigen is MUC16. In certain embodiments, the neoplasm is acute myeloid leukemia (AML).

Additionally, the presently disclosed subject matter provides novel mouse models. In certain embodiments, the mouse exhibits one or more cytokine release syndrome (CRS)-related symptom. In certain embodiments, the mouse comprises:

(a) a tumor cell;

(b) an immunoresponsive cell comprising an antigen-recognizing receptor that binds to an antigen, wherein the immunoresponsive cell is present in an amount sufficient to induce one or more CRS-related symptom.

In certain embodiments, the mouse is an immunocompetent mouse. In certain embodiments, the mouse is an immunodeficient mouse. In certain embodiments, the immunodeficient mouse is a SCID-beige mouse. In certain embodiments, the tumor cell is a human tumor cell or a murine tumor cell.

In certain embodiments, the mouse comprises at least about 10⁷ of the immunoresponsive cells. In certain embodiments, the mouse comprises at least about 10⁸ of the immunoresponsive cells. In certain embodiments, the immunoresponsive cell is a T cell. In certain embodiments, the antigen-recognizing receptor comprised in the immunoresponsive cell is a CAR.

In certain embodiments, the one or more CRS-related symptom is selected from the group consisting of elevated level of one or more pro-inflammatory cytokine, rapid weight loss, piloerection, reduced activity, general presentation of malaise, mortality and any combination thereof. In certain embodiments, the one or more CRS-related symptom is present about 12 hours after the introduction of the immunoresponsive cells to the mouse. In certain embodiments, the one or more pro-inflammatory cytokine is selected from the group consisting of IL-1 alpha, IL-1 beta, IL-6, IL-8, IL-10, TNF-α, and IFN-γ. In certain embodiments, the mouse does not exhibit Graft versus Host Disease (GvHD).

The presently disclosed subject matter further provides uses of the mouse model disclosed herein for screening an agent that is capable of preventing, alleviating and/or treating cytokine release syndrome (CRS). In certain embodiments, the method comprises: (a) administering a test agent to a mouse disclosed herein, and (b) measuring one or more CRS-related symptom in the mouse; and wherein alleviation of one or more CRS-related symptoms is indicates that the test agent is likely to be capable of preventing, alleviating and/or treating CRS. In certain embodiments, where the alleviation of one or more CRS-related symptoms comprises decreased level of one or more of pro-inflammatory cytokine, weight gain, reduced and/or eliminated piloerection, reduced and/or eliminated malaise, prolonged survival, or a combination thereof.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1T depict a mouse model of CRS recapitulating clinical features of the pathology. A) Schematic of mouse model. Raji tumor cells were intraperitoneally injected in mice and allowed to grow for three weeks. A high dose of CAR T cells was transferred, and mice were monitored over the following hours for symptoms of CRS. Mice were sacrificed, and cells were obtained for analysis through peritoneal lavage or tissue harvesting for further analysis. B) and Q) Percent weight change of tumor bearing mice after 1928z CAR T cell transfer. Weight per mouse was normalized to starting weight pre-CAR transfer (Tumor only n=12, Tumor+CAR n=18). C) and R) Percent survival of mice after 1928z CAR T cell transfer (Tumor only n=12, Tumor+CAR n=18). D) Serum levels of murine SAA3 at 42 hours post 1928z CAR T cell transfer as measured by ELISA (Baseline [tumor-free mice]/No tumor no CAR n=5, tumor only n=5, tumor+CAR n=5, CAR only n=5). E)-L) and S) Serum cytokine levels 4.5 hours before (pre-car) or 24 hours post 1928z CAR T cell transfer (CRS or Severe CRS). Mice that died from CRS were grouped under severe CRS while mice that survived but suffered greater than 10% weight loss were grouped under CRS. “m” prefix denotes murine while “h” prefix denotes human. Cytokine levels were measured by Cytokine Bead Array (CBA). M)-O) and T) Species of origin of pro-inflammatory cytokines. P) Percent survival of tumor bearing mice treated with 1928z CAR T cells that received murine IL-6R blocking antibody or isotype (vehicle). S) Serum levels of murine SAA3 at 42 hours post 1928z CART cell transfer as measured by ELISA (No tumor No CAR n=5, tumor only n=5, tumor+CAR n=5, CAR only n=5). *P<0.05, **P<0.01, ***P<0.001 (Two-way ANOVA (B); (two-tailed unpaired two-sample t-test was used; log-rank Mantel-Cox test (C and P). All data are means±s.e.m.

FIGS. 2A-2R depict that tumor−CAR T cell interactions selectively trigger myeloid cell recruitment and activation. A) and B) Immunohistochemical staining of sections from 3-week tumor explants for Mac2. C) and P) Absolute counts of myeloid cell populations obtained by peritoneal lavage 60 hours after 1928z CAR T cell transfer. Phenotypes were analyzed by flow cytometry and absolute quantification was performed by the addition of counting beads. (Baseline [tumor free mice]/No tumor no CAR n=5, CAR only n=5, Tumor only n=6, Tumor+CAR n=7). D) Representative flow cytometric plot showing Total Peritoneal Macrophages within the gated population [Resident Peritoneal Macrophages and CRS-Associated Macrophages (CAMs)]. Cells were obtained from peritoneal lavage. E)-G) and Q) Absolute counts of myeloid cell populations obtained from multiple organs 18 hours after 1928z CAR T cell transfer (Tumor only n=4, Tumor+CAR n=4). H)-O) and R) Fold change of pro-inflammatory gene expression in myeloid populations as determined by RNAseq analysis. Fold change was determined by comparing each population under tumor only and tumor+CAR conditions. Significant downregulation (green bars), significant upregulation (red bars), no significant change (gray bars). Gene expression levels were determined from three biological replicates for tumor only mice and three biological replicates for tumor+CAR mice. Each biological replicate consisted of pooled cells isolated from three mice. *P<0.05, **P<0.01, ***P<0.001 (Welch's two samples t-test (C and E-G); (binomial test, FDR-adjusted p-values (H-O). All data are means±s.e.m. except C-E which are means±s.d.

FIGS. 3A-3S depict that modulating macrophage function drastically alters CRS outcomes. A) Schematic of SFG retroviral cassette designed to co-express 1928z and murine CD40L. B) and M) Percent weight change of tumor bearing mice after 1928z CAR T cell transfer. Weight per mouse was normalized to starting weight pre-CAR transfer (Tumor only n=8, 1928z-LNGFR n=7, 1928z-mCD40L n=5). C) and D) Representative flow cytometric plot showing Total Peritoneal Macrophages within the gated population [Resident Peritoneal Macrophages and CRS-Associated Macrophages (CAMs)]. Cells were obtained from peritoneal lavage. E) and O) Percent of CD40⁺ total peritoneal macrophages, obtained by peritoneal lavage at 61 hours post 1928z-LNGFR or 1928z-mCD40L CAR T cell transfer, analyzed by flow cytometry. F)-I) and P) Serum levels of murine cytokines at 18 hours post CAR T cell transfer. Cytokine levels were measured by Cytokine Bead Array (CBA). (Tumor only n=8, 1928z-LNGFR n=7, 1928z-mCD40L n=5). J) and Q) Percent of myeloid populations from peritoneum, spleen and bone marrow expressing iNOS protein in tumor only mice and tumor+CAR mice. iNOS expression was determined by intracellular flow cytometry. (For peritoneum n=14 per group, for bone marrow and spleen n=10 per group). K) and R) Percent weight change of tumor bearing mice after 1928z CAR T cell transfer. Weight per mouse was normalized to starting weight pre-CAR transfer. Mice were treated with L-NIL or vehicle (PBS). (Tumor only n=7, CAR+L-NIL n=7, CAR+Vehicle n=8). L) and S) Percent survival of tumor bearing mice after 1928z CAR T cell transfer receiving 1400W or Vehicle (PBS). (Vehicle n=20, 1400W n=13). N) Percent survival of tumor bearing mice after 1928z CAR T cell transfer (1928z-LNGFR n=16, 1928z-mCD40L n=13). *P<0.05, **P<0.01, ***P<0.001 (Two-way ANOVA (B and K); (Two-tailed unpaired two-sample t-test was used; (log-rank Mantel-Cox test (I). All data are means±s.e.m.

FIGS. 4A-4R depict that augmented IL-1Ra response alleviated CRS-associated mortality without compromising antitumor efficacy. A)-H) Fold change of IL-1 signaling component gene expression in myeloid populations as determined by RNAseq analysis. Fold change was determined by comparing each population under tumor only and tumor+CAR conditions. Significant downregulation (green bars), significant upregulation (red bars), no significant change (grey bars). Gene expression levels were determined from three biological replicates for tumor only mice and three biological replicates for tumor+CAR mice. Each biological replicate consisted of pooled cells isolated from three mice. I) Percent survival of tumor bearing mice after 1928z CAR T cell transfer receiving Anakinra or Vehicle (PBS). (Anakinra n=11, Vehicle n=10). J) Percent of peritoneal macrophages expressing iNOS at 18 hours post CAR T cell transfer. Mice were treated with isotype, murine IL-6 blocking antibody, Anakinra or murine IL-6 blocking antibody+Anakinra. (Tumor only=4, Isotype n=3, Anti-mIL-6 n=3, Anakinra n=3, Anti-mIL-6+Anakinra n=4). K) Schematic of SFG retroviral cassette designed to co-express 1928z and murine IL-1Ra. L) Levels of murine IL-1Ra in supernatants of 1928z-LNGFR and 1928z-mIL-1Ra transduced CAR T cells after 48 hours in culture as determined by ELISA. M) Percent survival of tumor bearing mice after 1928z-LNGFR or 1928z-mIL-1Ra CAR T cell transfer. (1928z-LNGFR n=22, 1928z-mIL-1Ra n=18). N)-P) Serum levels of murine cytokines at 18 hours post CAR T cell transfer. Tumor bearing mice received 1928z-LNGFR or 1928z-mIL-1Ra CAR T cells. Cytokine levels were measured by Cytokine Bead Array (CBA). Q)-R) Percent tumor free survival of NSG mice receiving 0.2e6 or 0.5e6 1928z-LNGFR or 1928z-mIL-1Ra CAR T cells. Tumors were injected intravenously on Day-4 and CAR T cells on Day 0. (Tumor only n=4, 0.2e6 1928z-LNGFR n=7, 0.2e6 1928z-mIL-1Ra n=7, 0.5e6 1928z-LNGFR n=11, 0.5e6 1928z-mIL-1Ra n=11). *P<0.05, **P<0.01, ***P<0.001 (binomial test, FDR-adjusted p-values (A-H); (Two-tailed unpaired two-sample t-test and one-way ANOVA were used; log-rank Mantel-Cox test (I, M, Q and R). All data are means±s.e.m.

FIGS. 5A-5K depict cytokine levels and the effects to mouse tissues. A) and E) Serum levels of human and murine IFNγ at 18 hours post 1928z CAR T cell transfer as measured by Cytokine Bead Array (CBA) (n=6). B) and G) Serum of murine IL-6 levels at 18 hours post 1928z CAR T cell transfer as measured by Cytokine Bead Array (CBA). Mice were treated with a blocking antibody specific for the murine IL-6 receptor or isotype (Isotype, n=3, Anti-mIL-6R n=3). C) and H) Representative tissue sections stained with H&E obtained from mice sacrificed after 2 days or 5 days of 1928z CAR T cell transfer and respective controls. D) Serum cytokine levels after 24 hours of 1928z CART cell treatment (No tumor no CAR n=5, tumor only n=4, CAR only n=5, Tumor+CAR n=3). F) Serum of murine IL-15/IL-15R complex levels at 18 hours post 1928z CART cell transfer as measured by ELISA. All data are means±s.e.m. FIGS. 5I-5K depict representative tissue sections of mouse brains stained with H&E, obtained from tumor only or tumor+CAR treated mice one, two and five days after CAR T cell transfer. (Day 1: Tumor only n=2 mice, Tumor+CAR n=3 mice), (Day 2: Tumor only n=3 mice, Tumor+CAR n=3 mice), (Day 5: Tumor only n=3 mice, Tumor+CAR n=2 mice). Day 1, Day 2 and Day 5 mice were derived from three independent experiments. (I. top row) Coronal section of the skull and the brain at the level of the hippocampus (H) and thalamus (T). The space between the cranial vault and the cerebrum on the right image is artefactual. (I. bottom row) Detail of the hippocampus and its regions (CAL CA3, DG). A portion of the choroid plexus (Cp) of the ventricular system, the cerebral meninges (arrowhead), brain cortex (C) are shown. (J. top row) Coronal section of the brain at the level of the frontal lobes. (J. bottom row) Detail of the dorsal aspect of the cortex (C) including the meninges (arrowhead). (K. top row) Coronal section of the brain at the level of the striatum (S) and corpus callosum (Cc). (K. bottom row) Detail of the dorsal aspect of the cortex (C), including the cerebral meninges (arrowhead).

FIGS. 6A-6G depict myeloid cell and T cell populations in various tissues. A) and F) Percent weight change of tumor bearing or tumor free mice after 1928z CAR T cell transfer. Weight per mouse was normalized to starting weight pre-CAR transfer (Baseline [tumor free mice]/No tumor no CAR n=5, CAR only n=5, Tumor only n=6, Tumor+CAR n=7). B)-D) and G) Absolute counts of myeloid cell populations obtained from various organs 18 hours after 1928z CAR T cell transfer. Phenotypes were analyzed by flow cytometry and absolute quantification was performed by the addition of counting beads. (Tumor only n=4, Tumor+CAR n=4). E) Representative flow cytometric plots of T cell distribution in various tissues 18 hours after 1928z CART cell transfer. *P<0.05, **P<0.01, ***P<0.001 (Two-way ANOVA (A); (Two-tailed unpaired two-sample t-test (B-D). Data are means±s.e.m (A) and means±s.d. (B-D).

FIGS. 7A-7B depict gating strategy to phenotype and FACS sort myeloid populations. A) Gating strategy to phenotype and FACS sort myeloid populations in cells obtained from peritoneal lavage. B) Gating strategy to phenotype and FACS sort myeloid populations in cells obtained from murine spleens.

FIGS. 8A-8H depict effects of 1928z-LNGFR treatment and 1928z-mCD40L treatment. A) Flow cytometric histogram of T cells transduced with 1928z-LNGFR. B) Percent survival of tumor bearing mice treated with 1928z-LNGFR or 1928z-mCD40L CAR T cells. (Tumor only n=9, 1928z-LNGFR n=7, 1928z-mCD40L n=7). C) and F) Absolute counts of myeloid cell populations obtained by peritoneal lavage 61 hours after 1928z-LNGFR or 1928z-mCD40L CAR T cell transfer. Phenotypes were analyzed by flow cytometry and absolute quantification of cells was performed by the addition of counting beads. (Tumor only n=8, 1928z-LNGFR n=7, 1928z-mCD40L n=5). D) and G) Percent of CD40+ DCs, obtained by peritoneal lavage at 61 hours post 1928z-LNGFR or 1928z-mCD40L CAR T cell transfer, analyzed by flow cytometry. (Tumor only n=9, 1928z-LNGFR n=7, 1928z-mCD40L n=5). E) Representative flow cytometric plots of murine CD40 expression on the surface of the indicated myeloid populations. H) Representative flow cytometric plots of murine CD40 expression on the surface of the indicated myeloid populations. *P<0.05, **P<0.01, ***P<0.001 (Two-tailed unpaired two-sample t-test (C) and One-way ANOVA (D) were used. All data are means±s.e.m.

FIGS. 9A-9E. A) and C) Absolute counts of iNOS+ myeloid cell populations obtained by peritoneal lavage after 1928z CAR T cell transfer. iNOS expression was determined by intracellular flow cytometry and absolute quantification of cells was performed by the addition of counting beads. (Tumor only n=14, Tumor+CAR n=14). B) and D) Percent weight change of tumor bearing mice after 1928z CAR T cell transfer. Mice received 1400W or vehicle (PBS) Weight per mouse was normalized to starting weight pre-CAR transfer (Tumor only n=10, CAR+Vehicle n=8, CAR+1400W n=8). E) Percent peritoneal macrophages expressing iNOS at 18 hours post CAR T cell transfer. Mice were treated with isotype, murine IL-6 blocking antibody, or murine IL-1b blocking antibody. (Tumor only n=6, Isotype n=3, Anti-mIL-6 n=8, Anti mIL-1b n=4). *P<0.05, **P<0.01, ***P<0.001 (Two-tailed unpaired two-sample t-test (A); Two-way ANOVA (B); one-way ANOVA (E)) All data are means±s.e.m.

FIGS. 10A-10D. A) Flow cytometric histogram showing percentage of transduced CAR T cells with 1928z-LNGFR and 1928z-mIL-1Ra constructs prior to transfer to SCID-beige mice. B) Flow cytometric histogram showing percentage of transduced CAR T cells with 1928z-LNGFR and 1928z-mIL-1Ra constructs prior to transfer to NSG mice. C) and D) Tumor derived (NALM-6) bioluminescent signal from NSG mice receiving 0.2e6 or 0.5e6 1928z-LNGFR or 1928z-mIL-1Ra CAR T cells. Tumors were injected intravenously on Day-4 and CAR T cells on Day 0. (Tumor only: n=4, 0.2e6 1928z-LNGFR: n=7, 0.2e6 1928z-mIL-1Ra: n=7, 0.5e6 1928z-LNGFR: n=11, 0.5e6 1928z-mIL-1Ra: n=11).

### DETAILED DESCRIPTION OF THE INVENTION

The presently disclosed subject matter provides cells, including genetically modified immunoresponsive cells (e.g., T cells, NK cells, or CTL cells) comprising a combination of an antigen-recognizing receptor (e.g., TCR or CAR) and a secretable IL-1Ra polypeptide (e.g., an exogenous IL-1Ra polypeptide, or a nucleic acid encoding an IL-1Ra polypeptide). The presently disclosed subject matter also provides methods of using such cells for treating and/or preventing a neoplasm or other diseases/disorders, reducing tumor burden in a subject, lengthening survival of a subject having neoplasm (e.g., cancer), and/or treating or alleviating CRS in a subject who receives an immunotherapy. The presently disclosed subject matter is based, at least in part, on the discovery that a secretable IL-1Ra polypeptide alleviated cytokine release syndrome (CRS) in subjects receiving an immunotherapy (e.g., CAR-T cells).

The presently disclosed subject matter is at least based on the discovery of a novel genetic construct that allows to prevent and/or reduce the severity of CRS effectively without the requirement for external administration of pharmacological agents, by co-expressing a CAR and IL-1Ra (encoded by IL-1RN gene) in T cells. This approach takes advantage of the natural function of endogenous IL-1Ra. This novel genetic construct when introduced into T cells allows for the constitutive co-expression of both the CAR protein and the IL-1Ra protein. Treatment of mice that experience CRS, with the T cells comprising such genetic construct (e.g., 1928z-IL-1Ra CAR T cells) are protected from CRS-related mortality. Moreover, in a mouse model suitable to compare the long-term anti-tumor efficacy of different CAR constructs, T cells comprising such genetic construct (e.g., 1928z-IL-1Ra CAR T cells) have equivalent anti-tumor efficacy compared to their control counterparts (e.g., 1928z CAR T cells that do not co-express IL-1Ra). Therefore, the presently disclosed subject matter allows for CRS to be treated intrinsically by the CAR T cell itself without affecting anti-tumor efficacy, while removing the need external pharmacological intervention.

The novel genetic construct sets a paradigm of co-expression of immunomodulatory molecules from engineered T cells in order to prevent, mitigate and/or ameliorate toxicities inherent to CAR-T cell therapy. Moreover, the presently disclosed subject matter provides methods of conditionally co-expressing such immunomodulatory molecules in CAR-T cells by inducible promoters. Conditional co-expression in this context can be achieved through the use of specialized promoters that induce transcription only upon the binding of specific transcription factors. In addition, expression levels can be further adjusted by using constitutive promoters of known strength in order to achieve the desired levels of expression. Lastly, other cell types employed for immunotherapy, such as NK cells or macrophages, can be also engineered with such immunomodulatory molecules and be used alone or in combination with CAR-T cells.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold or within 2-fold, of a value.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3 γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, ICOS, and MyD88. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}:: V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest (e.g., an IL-1Ra polypeptide) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more e.g., at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., e.g., of at least about 42° C., e.g., of at least about 68° C. In certain embodiments, wash steps will occur at 25° C. in 30 mM NaCl, 3 mM trisodium citrate, and 0.1% SDS. In certain embodiments, wash steps occur at 42° C. in 15 mM NaCl, 1.5 mM trisodium citrate, and 0.1% SDS. In certain embodiments, wash steps occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm and/or CRS.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “immunoresponsive cell” is meant a cell that functions in an immune response or a progenitor, or progeny thereof.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises a sequence set forth in GGGGSGGGGSGGGGS [SEQ ID NO: 23].

By “neoplasm” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasia can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, the IL-2 signal sequence: MYRMQLLSCIALSLALVTNS [SEQ ID NO: 8] (human), MYSMQLASCVTLTLVLLVNS [SEQ ID NO: 24] (mouse); the kappa leader sequence: METPAQLLFLLLLWLPDTTG [SEQ ID NO: 25] (human), METDTLLLWVLLLWVPGSTG [SEQ ID NO: 26] (mouse); the CD8 leader sequence: MALPVTALLLPLALLLHAARP [SEQ ID NO: 27] (human); the albumin signal sequence: MKWVTFISLLFSSAYS [SEQ ID NO: 28] (human); and the prolactin signal sequence: MDSKGSSQKGSRLLLLLVVSNLLLCQGVVS [SEQ ID NO: 29] (human).

By “soluble” is meant a polypeptide that is freely diffusible in an aqueous environment (e.g., not membrane bound).

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-IS neoplastic cell. In certain embodiments, a tumor antigen includes any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 2. Antigen-Recognizing Receptors

The present disclosure provides antigen-recognizing receptors that bind to an antigen of interest. In certain embodiments, the antigen-recognizing receptor is a chimeric antigen receptor (CAR). In certain embodiments, the antigen-recognizing receptor is a T-cell receptor (TCR). The antigen-recognizing receptor can bind to a tumor antigen or a pathogen antigen.

### 2.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CA1X), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, ERBB, ITGB5, PTPRJ, SLC30A1, EMC10, SLC6A6, TNFRSF1B, CD82, ITGAX, CR1, DAGLB, SEMA4A, TLR2, LTB4R, P2RY13, LILRB2, EMB, CD96, LILRB3, LILRA6, LILRA2, ADGRE2, LILRB4, CD70, CCR1, CCR4, TACT, TRBC1, and TRBC2.

In certain embodiments, the antigen-recognizing receptor binds to CD19. In certain embodiments, the antigen-recognizing receptor binds to a murine CD19 polypeptide. In certain embodiments, the antigen-recognizing receptor binds to a human CD19 polypeptide. In certain embodiments, the antigen-recognizing receptor binds to exon 2 of CD19.

In certain embodiments, the antigen-recognizing receptor binds to an AML antigen. Non-limiting examples of AML antigens disclosed in WO2018027197, which is incorporated by reference in its entirety.

In certain embodiments, the antigen-recognizing receptor binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a second generation CAR.

In certain non-limiting embodiments, the extracellular antigen-binding domain of the CAR (embodied, for example, an scFv or an analog thereof) binds to an antigen with a dissociation constant (K_{d}) of about 5×10⁻⁶ M or less. In certain embodiments, the K_{d} is about 5×10⁻⁶ M or less, about 1×10⁻⁶ M or less, 5×10⁻⁷ M or less, about 2×10⁻⁷ M or less, about 1×10⁻⁷ M or less, about 9×10⁻⁸M or less, about 1×10⁻⁸M or less, about 9×10⁻⁹M or less, about 5×10⁻⁹M or less, about 4×10⁻⁹M or less, about 3×10⁻⁹ or less, about 2×10⁻⁹ M or less, or about 1×10⁻⁹M or less, or about 1×10⁻¹⁰ M or less. In certain non-limiting embodiments, the K_{d} is about 1×10⁻⁹M or less. In certain non-limiting embodiments, the K_{d} is about 1×10⁻¹⁰ M or less. In certain non-limiting embodiments, the K_{d} is from about 1×10⁻¹⁰ M to about 1×10⁻⁶ M. In certain non-limiting embodiments, the K_{d} is from about 1×10⁻⁹M to about 1×10⁻⁷ M. In certain non-limiting embodiments, the K_{d} is from about 1×10⁻¹⁰ M to about 1×10⁻⁷ M. In certain non-limiting embodiments, the K_{d} is from about 1×10⁻⁹M to about 1×10⁻⁷ M.

Binding of the extracellular antigen-binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

In accordance with the presently disclosed subject matter, a CARs can comprise an extracellular antigen-binding domain, a transmembrane domain and an intracellular signaling domain, wherein the extracellular antigen-binding domain specifically binds to an antigen, e.g., a tumor antigen or a pathogen antigen.

2.3.1. Extracellular Antigen Binding Domain of A CAR

In certain embodiments, the extracellular antigen-binding domain specifically binds to an antigen. In certain embodiments, the extracellular antigen-binding domain is an scFv. In certain embodiments, the scFv is a human scFv, a humanized scFv, or a murine scFv. In certain embodiments, the extracellular antigen-binding domain is a Fab, which is optionally crosslinked. In certain embodiments, the extracellular antigen-binding domain is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. In certain embodiments, the antigen is a tumor antigen. In certain embodiments, the antigen is a pathogen antigen.

In certain embodiments, the extracellular antigen-binding domain of a presently disclosed CAR is a murine scFv. In certain embodiments, the extracellular antigen-binding domain of a presently disclosed CAR is a murine scFv that binds to a murine CD19 polypeptide.

In certain embodiments, the extracellular antigen-binding domain of a presently disclosed CAR is an scFv that binds to a human CD19 polypeptide. In certain embodiments, the extracellular antigen-binding domain is a murine scFv, which comprises the amino acid sequence of SEQ ID NO: 6 and specifically binds to a human CD19 polypeptide. In certain embodiments, the nucleotide sequence encoding the amino acid sequence of SEQ ID NO: 6 is set forth in SEQ ID NO: 7. In certain embodiments, the murine scFv comprises a heavy chain variable region (V_{H}) comprising the amino acid sequence set forth in SEQ ID NO: 54. In certain embodiments, the murine scFV comprises a light chain variable region (V_{L}) comprising the amino acid sequence set forth in SEQ ID NO: 55. In certain embodiments, the murine scFV comprises V_{H} comprising the amino acid sequence set forth in SEQ ID NO: 54 and a V_{L} comprising the amino acid sequence set forth in SEQ ID NO: 55, optionally with (iii) a linker sequence, for example a linker peptide, between the V_{H} and the V_{L}. In certain embodiments, the linker comprises amino acids having the sequence set forth in SEQ ID NO: 23. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 54. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 54. In certain embodiments, the extracellular antigen-binding domain comprises a V_{H} comprising the amino sequence set forth in SEQ ID NO: 54. In certain embodiments, the extracellular antigen-binding domain comprises a V_{L} comprising an amino acid sequence that is at least about 80% (e.g., at least about 85%, at least about 90%, or at least about 95%) homologous to SEQ ID NO: 55. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous to SEQ ID NO: 55. In certain embodiments, the extracellular antigen-binding domain comprises a V_{L} comprising the amino acid sequence set forth in SEQ ID NO: 55. In certain embodiments, the extracellular antigen-binding domain comprises a V_{H} comprising an amino acid sequence that is at least about 80% (e.g., at least about 85%, at least about 90%, or at least about 95%) homologous to SEQ ID NO: 54, and a V_{L} comprising an amino acid sequence that is at least about 80% (e.g., at least about 85%, at least about 90%, or at least about 95%) homologous to SEQ ID NO: 55. In certain embodiments, the extracellular antigen-binding domain comprises a V_{H} comprising the amino acid sequence set forth in SEQ ID NO: 54 and a V_{L} comprising the amino acid sequence set forth in SEQ ID NO: 55. In certain embodiments, the extracellular antigen-binding domain comprises a V_{H} CDR1 comprising the amino acid sequence set forth in SEQ ID NO: 48, or a conservative modification thereof, a V_{H} CDR2 comprising the amino acid sequence set forth in SEQ ID NO: 49 or a conservative modification thereof, and a V_{H} CDR3 comprising the amino acid sequence set forth in SEQ ID NO: 50, a conservative modification thereof. In certain embodiments, the extracellular antigen-binding domain comprises a V_{H} CDR1 comprising the amino acid sequence set forth in SEQ ID NO: 48, a V_{H} CDR2 comprising the amino acid sequence set forth in SEQ ID NO: 49, and a V_{H} CDR3 comprising the amino acid sequence set forth in SEQ ID NO: 50. In certain embodiments, the extracellular antigen-binding domain comprises a V_{L} CDR1 comprising the amino acid sequence set forth in SEQ ID NO: 51 or a conservative modification thereof, a V_{L} CDR2 comprising the amino acid sequence set forth in SEQ ID NO: 52 or a conservative modification thereof, and a V_{L} CDR3 comprising the amino acid sequence set forth in SEQ ID NO: 53 or a conservative modification thereof. In certain embodiments, the extracellular antigen-binding domain comprises a V_{L} CDR1 comprising the amino acid sequence set forth in SEQ ID NO: 51, a V_{L} CDR2 comprising the amino acid sequence set forth in SEQ ID NO: 52, and a V_{L} CDR3 comprising the amino acid sequence set forth in SEQ ID NO: 53. In certain embodiments, the extracellular antigen-binding domain comprises a V_{H} CDR1 comprising the amino acid sequence set forth in SEQ ID NO: 48 or a conservative modification thereof, a V_{H} CDR2 comprising the amino acid sequence set forth in SEQ ID NO: 49 or a conservative modification thereof, a V_{H} CDR3 comprising the amino acid sequence set forth in SEQ ID NO: 50, a conservative modification thereof, a V_{L} CDR1 comprising the amino acid sequence set forth in SEQ ID NO: 51 or a conservative modification thereof, a V_{L} CDR2 comprising the amino acid sequence set forth in SEQ ID NO: 52 or a conservative modification thereof, and a V_{L} CDR3 comprising the amino acid sequence set forth in SEQ ID NO: 53 or a conservative modification thereof. In certain embodiments, the extracellular antigen-binding domain comprises a V_{H} CDR1 comprising amino acids having the sequence set forth in SEQ ID NO: 48, a V_{H} CDR2 comprising the amino acid sequence set forth in SEQ ID NO: 49, a V_{H} CDR3 comprising the amino acid sequence set forth in SEQ ID NO: 50, a V_{L} CDR1 comprising the amino acid sequence set forth in SEQ ID NO: 51, a V_{L} CDR2 comprising the amino acid sequence set forth in SEQ ID NO: 52 and a V_{L} CDR3 comprising the amino acid sequence set forth in SEQ ID NO: 53. SEQ ID NOS: 6, 7 and 43 to 58 are provided in Table 1.

TABLE 1

| | | Mouse anti-human CD19 scFv | | |
|---|---|---|---|---|
| CDRs | | 1 | 2 | 3 |
| V_{H} | a.a. | GYAFSSY | YPGDGD | KTISSVVDFYFDY |
| | | [SEQ ID NO: 48] | [SEQ ID NO: 49] | [SEQ ID NO: 50] |
| | nt | Ggctatgcattcagta | Tatcctggagatggtga | Aagaccattagttcggtag |
| | | gctac | t [SEQ ID NO: 44] | tagatttctactttgacta |
| | | [SEQ ID NO: 43] | | c [SEQ ID NO: 45] |
| V_{L} | a.a. | KASQNVGTNVA | SATYRNS | QQYNRYPYT |
| | | [SEQ ID NO: 51] | [SEQ ID NO: 52] | [SEQ ID NO: 53] |
| | nt | Aaggccagtcagaatg | Tcggcaacctaccggaa | Caacaatataacaggtatc |
| | | tgggtactaatgtagc | cagt | cgtacacg |
| | | c | [SEQ ID NO: 47] | [SEQ ID NO: 56] |
| | | [SEQ ID NO: 46] | | |

| | | |
|---|---|---|
| Full | a.a. | EVKLQQSGAELVRPGSSVKISCKASGYAFSSYWMNWVKQRPGQGLEWIGQIYPGDG |
| V_{H} | | DTNYNGKFKGQATLTADKSSSTAYMQLSGLTSEDSAVYFCARKTISSVVDFYFDYW |
| | | GQGTTVTVSS [SEQ ID NO: 54] |
| | nt | Gaggtgaagctgcagcagtctggggctgagctggtgaggcctgggtcctcagtgaa |
| | | gatttcctgcaaggcttctggctatgcattcagtagctactggatgaactgggtga |
| | | agcagaggcctggacagggtcttgagtggattggacagatttatcctggagatggt |
| | | gatactaactacaatggaaagttcaagggtcaagccacactgactgcagacaaatc |
| | | ctccagcacagcctacatgcagctcagcggcctaacatctgaggactctgcggtct |
| | | atttctgtgcaagaaagaccattagttcggtagtagatttctactttgactactgg |
| | | ggccaagggaccacggtcaccgtctcctca [SEQ ID NO: 57] |
| Full | a.a. | DIELTQSPKFMSTSVGDRVSVTCKASQNVGTNVAWYQQKPGQSPKPLIYSATYRNS |
| V_{L} | | GVPDRFTGSGSGTDFTLTITNVQSKDLADYFCQQYNRYPYTSGGGTKLEIKR |
| | | [SEQ ID NO: 55] |
| | nt | Gacattgagctcacccagtctccaaaattcatgtccacatcagtaggagacagggt |
| | | cagcgtcacctgcaaggccagtcagaatgtgggtactaatgtagcctggtatcaac |
| | | agaaaccaggacaatctcctaaaccactgatttactcggcaacctaccggaacagt |
| | | ggagtccctgatcgcttcacaggcagtggatctgggacagatttcactctcaccat |
| | | cactaacgtgcagtctaaagacttggcagactatttctgtcaacaatataacaggt |
| | | atccgtacacgtccggaggggggaccaagctggagatcaaacgg |
| | | [SEQ ID NO: 58] |
| scFv | a.a. | MALPVTALLLPLALLLHAEVKLQQSGAELVRPGSSVKISCKASGYAFSSYWMNWVK |
| (in- | | QRPGQGLEWIGQIYPGDGDTNYNGKFKGQATLTADKSSSTAYMQLSGLTSEDSAVY |
| cluding | | FCARKTISSVVDFYFDYWGQGTTVTVSSGGGGSGGGGSGGGGSDIELTQSPKFMST |
| a CD8a | | SVGDRVSVTCKASQNVGTNVAWYQQKPGQSPKPLIYSATYRNSGVPDRFTGSGSGT |
| leader | | DFTLTITNVQSKDLADYFCQQYNRYPYTSGGGTKLEIKR [SEQ ID NO: 6] |
| sequence | nt | Atggctctcccagtgactgccctactgcttcccctagcgcttctcctgcatgcaga |
| | | ggtgaagctgcagcagtctggggctgagctggtgaggcctgggtcctcagtgaaga |
| | | tttcctgcaaggcttctggctatgcattcagtagctactggatgaactgggtgaag |
| | | cagaggcctggacagggtcttgagtggattggacagatttatcctggagatggtga |
| | | tactaactacaatggaaagttcaagggtcaagccacactgactgcagacaaatcct |
| | | ccagcacagcctacatgcagctcagcggcctaacatctgaggactctgcggtctat |
| | | ttctgtgcaagaaagaccattagttcggtagtagatttctactttgactactgggg |
| | | ccaagggaccacggtcaccgtctcctcaggtggaggtggatcaggtggaggtggat |
| | | ctggtggaggtggatctgacattgagctcacccagtctccaaaattcatgtccaca |
| | | tcagtaggagacagggtcagcgtcacctgcaaggccagtcagaatgtgggtactaa |
| | | tgtagcctggtatcaacagaaaccaggacaatctcctaaaccactgatttactcgg |
| | | caacctaccggaacagtggagtccctgatcgcttcacaggcagtggatctgggaca |
| | | gatttcactctcaccatcactaacgtgcagtctaaagacttggcagactatttctg |
| | | tcaacaatataacaggtatccgtacacgtccggaggggggaccaagctggagatca |
| | | aacgg [SEQ ID NO: 7] |

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology to a specific sequence (e.g., SEQ ID NOs: 54 and 55) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 54 and 55). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from the group consisting of SEQ ID NOs: 54 and 55, including post-translational modifications of that sequence (SEQ ID NO: 54 and 55).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used.

2.3.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a synthetic peptide (not based on a protein associated with the immune response), or a combination thereof.

In certain embodiments, the transmembrane domain comprises a CD8 polypeptide. In certain embodiments, the CD8 polypeptide comprises or has an amino acid sequence that is at least about 85%, about 90%, about 95%, about 96%, about 97%, about 98%, about 99% or about 100% homologous to the sequence having a NCBI Reference No: NP_001139345.1 (SEQ ID NO: 9) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 9 which is at least 20, or at least 30, or at least 40, or at least 50, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 150 to 200, or 200 to 235 of SEQ ID NO: 9. In certain embodiments, the CAR of the presently disclosed comprises a transmembrane domain comprising a CD8 polypeptide that comprises or has an amino acid sequence of amino acids 137 to 209 of SEQ ID NO: 9.

[SEQ ID NO: 9]
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

In certain embodiments, the CD8 polypeptide comprises or has an amino acid sequence that is at least about 85%, about 90%, about 95%, about 96%, about 97%, about 98%, about 99% or about 100% homologous to the sequence having a NCBI Reference No: AAA92533.1 (SEQ ID NO: 10) (homology herein may be determined using standard software such as BLAST or FASTA) as provided below, or fragments thereof, and/or may optionally comprise up to one or up to two or up to three conservative amino acid substitutions. In certain embodiments, the CD8 polypeptide comprises or has an amino acid sequence that is a consecutive portion of SEQ ID NO: 10 which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 10. In certain embodiments, the CAR of the presently disclosed comprises a transmembrane domain comprising a CD8 polypeptide that comprises or has an amino acid seauence of amino acids 151 to 219 of SEQ ID NO: 10.

| [SEQ ID NO: 10] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the CD8 polypeptide comprises or has the amino acid sequence set forth in SEQ ID NO: 11, which is provided below:

[SEQ ID NO: 11]
STTTKPVLRTPSPVHPTGTSQPQRPEDCRPRGSVKGTGLDFACDIYIWAP
LAGICVALLLSLIITLICY

In accordance with the presently disclosed subject matter, a “CD8 nucleic acid molecule” refers to a polynucleotide encoding a CD8 polypeptide.

In certain embodiments, the CD8 nucleic acid molecule encoding the CD8 polypeptide having the amino acid sequence set forth in SEQ ID NO: 11 comprises or has nucleic acids having the sequence set forth in SEQ ID NO: 12 as provided below.

[SEQ ID NO: 12]
TCTACTACTACCAAGCCAGTGCTGCGAACTCCCTCACCTGTGCACCCTAC
CGGGACATCTCAGCCCCAGAGACCAGAAGATTGTCGGCCCCGTGGCTCAG
TGAAGGGGACCGGATTGGACTTCGCCTGTGATATTTACATCTGGGCACCC
TTGGCCGGAATCTGCGTGGCCCTTCTGCTGTCCTTGATCATCACTCTCAT
CTGCTAC

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: P10747 or NP 006130 (SEQ ID NO: 2), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 2. In certain embodiments, the CD28 polypeptide comprised in the transmembrane domain of a presently disclosed CAR comprises or has an amino acid sequence of amino acids 153 to 179 of SEQ ID NO: 2.

SEQ ID NO: 2 is provided below:

| [SEQ ID NO: 2] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide having amino acids 153 to 179 of SEQ ID NO: 2 comprises or has nucleic acids having the sequence set forth in SEQ ID NO: 22 as provided below.

[SEQ ID NO: 22]
ttttgggtgctggtggtggttggtggagtcctggcttgctatagcttgct
agtaacagtggcctttattattttctgggtg

In certain embodiments, the intracellular signaling domain of the CAR comprises a human CD28 transmembrane domain. The human CD28 transmembrane domain can comprise or have an amino acid sequence that is at least about 85%, about 90%, about 95%, about 96%, about 97%, about 98%, about 99% or about 100% homologous to SEQ ID NO: 34 or fragments thereof, and/or may optionally comprise up to one or up to two or up to three conservative amino acid substitutions. SEQ ID NO: 34 is provided below:

| | |
|---|---|
| FWVLVVVGGV LACYSLLVTV AFIIFWV. | [SEQ ID NO: 34] |

An exemplary nucleic acid sequence encoding the amino acid sequence of SEQ ID NO: 34 is set forth in SEQ ID NO: 35, which is provided below.

[SEQ ID NO: 35]
TTTTGGGTGCTGGTGGTGGTTGGTGGAGTCCTGGCTTGCTATAGCTTGCT
AGTAACAGTGGCCTTTATTATTTTCTGGGTG

In certain non-limiting embodiments, a CAR can also comprise a spacer region that links the extracellular antigen-binding domain to the transmembrane domain. The spacer region can be flexible enough to allow the antigen binding domain to orient in different directions to facilitate antigen recognition. The spacer region can be the hinge region from IgG1, or the CH₂CH₃ region of immunoglobulin and portions of CD3, a portion of a CD28 polypeptide (e.g., a portion of SEQ ID NO: 2), a portion of a CD8 polypeptide (e.g., a portion of SEQ ID NO: 9, or a portion of SEQ ID NO: 10), a variation of any of the foregoing which is at least about 80%, at least about 85%, at least about 90%, or at least about 95% homologous thereto, or a synthetic spacer sequence.

2.3.3. Intracellular Signaling Domain of a CAR

In certain non-limiting embodiments, an intracellular signaling domain of the CAR comprises a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The intracellular signaling domain of the CD3ζ-chain is the primary transmitter of signals from endogenous TCRs. In certain embodiments, the CD3ζ polypeptide comprises or has an amino acid sequence that is at least about 85%, about 90%, about 95%, about 96%, about 97%, about 98%, about 99% or about 100% homologous to the sequence having a NCBI Reference No: NP_932170 (SEQ ID NO: 1), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 1, which is at least 20, or at least 30, or at least 40, or at least 50, and up to 164 amino acids in length. Alternatively or additionally, in non-limiting various embodiments, the CD3ζ polypeptide comprises or has an amino acid sequence of amino acids 1 to 164, 1 to 50, 50 to 100, 100 to 150, or 150 to 164 of SEQ ID NO: 1. In certain embodiments, the CD3ζ polypeptide comprises or has an amino acid sequence of amino acids 52 to 164 of SEQ ID NO: 1.

SEQ ID NO: 1 is provided below:

| [SEQ ID NO: 1] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the CD3ζ polypeptide comprises or has an amino acid sequence that is at least about 85%, about 90%, about 95%, about 96%, about 97%, about 98%, about 99% or about 100% homologous to the sequence having a NCBI Reference No: NP 001106864.2 (SEQ ID NO: 13), or fragments thereof, and/or may optionally comprise up to one or up to two or up to three conservative amino acid substitutions. In certain non-limiting embodiments, the CD3ζ polypeptide comprises or has an amino acid sequence that is a consecutive portion of SEQ ID NO: 13, which is at least about 20, or at least about 30, or at least about 40, or at least about 50, or at least about 90, or at least about 100, and up to 188 amino acids in length. Alternatively or additionally, in non-limiting various embodiments, the CD3ζ polypeptide comprises or has an amino acid sequence of amino acids 1 to 164, 1 to 50, 50 to 100, 52 to 142, 100 to 150, or 150 to 188 of SEQ ID NO: 13. In certain embodiments, the CD3ζ polypeptide comprises or has an amino acid sequence of amino acids 52 to 142 of SEQ ID NO: 13.

SEQ ID NO: 13 is provided below:

| [SEQ ID NO: 13] | |
|---|---|
| 1 | MKWKVSVLAC ILHVRFPGAE AQSFGLLDPK LCYLLDGILF IYGVIITALY LRAKFSRSAE |
| 61 | TAANLQDPNQ LYNELNLGRR EEYDVLEKKR ARDPEMGGKQ RRRNPQEGVY NALQKDKMAE |
| 121 | AYSEIGTKGE RRRGKGHDGL YQDSHFQAVQ FGNRREREGS ELTRTLGLRA RPKACRHKKP |
| 181 | LSLPAAVS |

In certain embodiments, the CD3ζ polypeptide comprises or has the amino acid sequence set forth in SEQ ID NO: 14, which is provided below:

[SEQ ID NO: 14]
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide having the amino acid sequence set forth in SEQ ID NO: 14 comprises or has the nucleotide sequence set forth in SEQ ID NO: 15 as provided below.

[SEQ ID NO: 15]
AGAGCAAAATTCAGCAGGAGTGCAGAGACTGCTGCCAACCTGCAGGACCC
CAACCAGCTCTACAATGAGCTCAATCTAGGGCGAAGAGAGGAATATGACG
TCTTGGAGAAGAAGCGGGCTCGGGATCCAGAGATGGGAGGCAAACAGCAG
AGGAGGAGGAACCCCCAGGAAGGCGTATACAATGCACTGCAGAAAGACAA
GATGGCAGAAGCCTACAGTGAGATCGGCACAAAAGGCGAGAGGCGGAGAG
GCAAGGGGCACGATGGCCTTTACCAGGGTCTCAGCACTGCCACCAAGGAC
ACCTATGATGCCCTGCATATGCAGACCCTGGCCCCTCGCTAA

In certain embodiments, the intracellular signaling domain of the CAR comprises a human CD3ζ polypeptide. The human CD3ζ polypeptide can comprise or have an amino acid sequence that is at least about 85%, about 90%, about 95%, about 96%, about 97%, about 98%, about 99% or about 100% homologous to SEQ ID NO: 32 or fragments thereof, and/or may optionally comprise up to one or up to two or up to three conservative amino acid substitutions. SEQ ID NO: 32 is provided below:

[SEQ ID NO: 32]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR.

An exemplary nucleic acid sequence encoding the amino acid sequence of SEQ ID NO: 32 is set forth in SEQ ID NO: 33, which is provided below.

[SEQ ID NO: 33]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC

In certain non-limiting embodiments, an intracellular signaling domain of the CAR further comprises at least a co-stimulatory signaling region. In certain embodiments, the co-stimulatory region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR′ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide. The CD28 polypeptide can comprise or have an amino acid sequence that is at least about 85%, about 90%, about 95%, about 96%, about 97%, about 98%, about 99% or 100% homologous to the sequence having a NCBI Reference No: P10747 or NP 006130 (SEQ ID NO: 2), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 2 which is at least 20, or at least 30, or at least 40, or at least 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 114 to 220, 150 to 200, or 200 to 220 of SEQ ID NO: 2. In certain embodiments, the intracellular signaling domain of the CAR comprises a co-stimulatory signaling region that comprises a CD28 polypeptide comprising or having an amino acid sequence of amino acids 180 to 220 of SEQ ID NO: 2.

In certain embodiments, the CD28 polypeptide comprises or has an amino acid sequence that is at least about 85%, about 90%, about 95%, about 96%, about 97%, about 98%, about 99% or about 100% homologous to the sequence having a NCBI Reference No: NP 031668.3 (SEQ ID NO: 16), or fragments thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 16 which is at least about 20, or at least about 30, or at least about 40, or at least about 50, and up to 218 amino acids in length. Alternatively or additionally, in non-limiting various embodiments, the CD28 polypeptide comprises or has an amino acid sequence of amino acids 1 to 218, 1 to 50, 50 to 100, 100 to 150, 114 to 220, 150 to 200, 178 to 218, or 200 to 220 of SEQ ID NO: 16. In certain embodiments, the co-stimulatory signaling region of a presently disclosed CAR comprises a CD28 polypeptide that comprises or has the amino acids 178 to 218 of SEQ ID NO: 16.

SEQ ID NO: 16 is provided below:

| [SEQ ID NO: 16] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. In certain embodiments, a CD28 nucleic acid molecule that encodes a CD28 polypeptide comprised in the co-stimulatory signaling region of a presently disclosed CAR (e.g., amino acids 178 to 218 of SEQ ID NO: 16) comprises or has a nucleotide sequence set forth in SEQ ID NO: 17, which is provided below.

[SEQ ID NO: 17]
AATAGTAGAAGGAACAGACTCCTTCAAAGTGACTACATGAACATGACTCC
CCGGAGGCCTGGGCTCACTCGAAAGCCTTACCAGCCCTACGCCCCTGCCA
GAGACTTTGCAGCGTACCGCCCC

In certain embodiments, the intracellular signaling domain of the CAR comprises a human intracellular signaling domain of CD28. The human intracellular signaling domain of CD28 can comprise or have an amino acid sequence that is at least about 85%, about 90%, about 95%, about 96%, about 97%, about 98%, about 99% or about 100% homologous to SEQ ID NO: 30 or fragments thereof, and/or may optionally comprise up to one or up to two or up to three conservative amino acid substitutions. SEQ ID NO: 30 is provided below:

[SEQ ID NO: 30]
RSKRSRLLHS DYMNMTPRRP GPTRKHYQPY APPRDFAAYR S.

An exemplary nucleic acid sequence encoding the amino acid sequence of SEQ ID NO: 30 is set forth in SEQ ID NO: 31, which is provided below.

[SEQ ID NO: 31]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the CAR comprises a human intracellular signaling domain of CD28. The human intracellular signaling domain of CD28 can comprise or have an amino acid sequence that is at least about 85%, about 90%, about 95%, about 96%, about 97%, about 98%, about 99% or about 100% homologous to SEQ ID NO: 30 or fragments thereof, and/or may optionally comprise up to one or up to two or up to three conservative amino acid substitutions. SEQ ID NO: 36 is provided below:

[SEQ ID NO: 36]
AIEVMYPPPYLDNEKSNGTIIHVKGKHLCPSPLFPGPSKPFWVLVVVGGV
LACYSLLVTVAFIIFWVRSKRSRLLHSDYMNMTPRRPGPTRKHYQPYAPP
RDFAAYRS.

An exemplary nucleic acid sequence encoding the amino acid sequence of SEQ ID NO: 36 is set forth in SEQ ID NO: 37, which is provided below.

[SEQ ID NO: 37]
GCAATTGAAGTTATGTATCCTCCTCCTTACCTAGACAATGAGAAGAGCAA
TGGAACCATTATCCATGTGAAAGGGAAACACCTTTGTCCAAGTCCCCTAT
TTCCCGGACCTTCTAAGCCCTTTTGGGTGCTGGTGGTGGTTGGTGGAGTC
CTGGCTTGCTATAGCTTGCTAGTAACAGTGGCCTTTATTATTTTCTGGGT
GAGGAGTAAGAGGAGCAGGCTCCTGCACAGTGACTACATGAACATGACTC
CCCGCCGCCCCGGGCCCACCCGCAAGCATTACCAGCCCTATGCCCCACCA
CGCGACTTCGCAGCCTATCGCTCC

In certain embodiments, the intracellular signaling domain of the CAR comprises a co-stimulatory signaling region that comprises two co-stimulatory molecules: CD28 and 4-1BB or CD28 and OX40.

4-1BB can act as a tumor necrosis factor (TNF) ligand and have stimulatory activity. The 4-1BB polypeptide can comprise or have an amino acid sequence that is at least about 85%, about 90%, about 95%, about 96%, about 97%, about 98%, about 99% or about 100% homologous to the sequence having a NCBI Reference No: P41273 or NP 001552 (SEQ ID NO: 3) or fragments thereof, and/or may optionally comprise up to one or up to two or up to three conservative amino acid substitutions.

SEQ ID NO: 3 is provided below:

| [SEQ ID NO: 3] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

In accordance with the presently disclosed subject matter, a “4-1BB nucleic acid molecule” refers to a polynucleotide encoding a 4-1BB polypeptide.

An OX40 polypeptide can comprise or have an amino acid sequence that is at least about 85%, about 90%, about 95%, about 96%, about 97%, about 98%, about 99% or about 100% homologous to the sequence having a NCBI Reference No: P43489 or NP_003318 (SEQ ID NO: 18), or fragments thereof, and/or may optionally comprise up to one or up to two or up to three conservative amino acid substitutions.

SEQ ID NO: 18 is provided below:

| [SEQ ID NO: 18] | |
|---|---|
| 1 | MCVGARRLGR GPCAALLLLG LGLSTVTGLH CVGDTYPSND RCCHECRPGN GMVSRCSRSQ |
| 61 | NTVCRPCGPG FYNDVVSSKP CKPCTWCNLR SGSERKQLCT ATQDTVCRCR AGTQPLDSYK |
| 121 | PGVDCAPCPP GHFSPGDNQA CKPWTNCTLA GKHTLQPASN SSDAICEDRD PPATQPQETQ |
| 181 | GPPARPITVQ PTEAWPRTSQ GPSTRPVEVP GGRAVAAILG LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

An ICOS polypeptide can comprise or have an amino acid sequence that is at least about 85%, about 90%, about 95%, about 96%, about 97%, about 98%, about 99% or about 100% homologous to the sequence having a NCBI Reference No: NP_036224 (SEQ ID NO: 19) or fragments thereof, and/or may optionally comprise up to one or up to two or up to three conservative amino acid substitutions.

SEQ ID NO: 19 is provided below:

| [SEQ ID NO: 19] | |
|---|---|
| 1 | MKSGLWYFFL FCLRIKVLTG EINGSANYEM FIFHNGGVQI LCKYPDIVQQ FKMQLLKGGQ |
| 61 | ILCDLIKTKG SGNTVSIKSL KFCHSQLSNN SVSFFLYNLD HSHANYYFCN LSIFDPPPFK |
| 121 | VTLTGGYLHI YESQLCCQLK FWLPIGCAAF VVVCILGCIL ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, a presently disclosed CAR comprises an extracellular antigen-binding domain that binds to CD19 (e.g., human CD19), a transmembrane domain comprising a CD28 polypeptide (e.g., human CD28 polypeptide), and an intracellular signaling domain comprising a CD3ζ polypeptide (e.g., a human CD3ζ polypeptide), wherein the intracellular signaling domain comprises a co-stimulatory signaling region, namely, the CAR is a second generation CAR. In certain embodiments, the CAR is designated as “1928Z”. In certain embodiments, the CAR (e.g., 1928Z) comprises an amino acid sequence that is at least about 85%, about 90%, about 95%, about 96%, about 97%, about 98%, about 99% or about 100% homologous to the amino acid sequence set forth in SEQ ID NO: 5, which is provided below. SEQ ID NO: 5 includes a CD8 leader sequence at amino acids 1 to 18, and is able to bind to CD19 (e.g., human CD19).

[SEQ ID NO: 5]
MALPVTALLLPLALLLHAEVKLQQSGAELVRPGSSVKISCKASGYAFSSY
WMNWVKQRPGQGLEWIGQIYPGDGDTNYNGKFKGQATLTADKSSSTAYMQ
LSGLTSEDSAVYFCARKTISSVVDFYFDYWGQGTTVTVSSGGGGSGGGGS
GGGGSDIELTQSPKFMSTSVGDRVSVTCKASQNVGTNVAWYQQKPGQSPK
PLIYSATYRNSGVPDRFTGSGSGTDFTLTITNVQSKDLADYFCQQYNRYP
YTSGGGTKLEIKRAAAIEVMYPPPYLDNEKSNGTIIHVKGKHLCPSPLFP
GPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR

An exemplary nucleic acid sequence encoding the amino acid sequence of SEQ ID NO: 5 is set forth in SEQ ID NO: 20, which is provided below.

[SEQ ID NO: 20]
ATGGCTCTCCCAGTGACTGCCCTACTGCTTCCCCTAGCGCTTCTCCTGCA
TGCAGAGGTGAAGCTGCAGCAGTCTGGGGCTGAGCTGGTGAGGCCTGGGT
CCTCAGTGAAGATTTCCTGCAAGGCTTCTGGCTATGCATTCAGTAGCTAC
TGGATGAACTGGGTGAAGCAGAGGCCTGGACAGGGTCTTGAGTGGATTGG
ACAGATTTATCCTGGAGATGGTGATACTAACTACAATGGAAAGTTCAAGG
GTCAAGCCACACTGACTGCAGACAAATCCTCCAGCACAGCCTACATGCAG
CTCAGCGGCCTAACATCTGAGGACTCTGCGGTCTATTTCTGTGCAAGAAA
GACCATTAGTTCGGTAGTAGATTTCTACTTTGACTACTGGGGCCAAGGGA
CCACGGTCACCGTCTCCTCAGGTGGAGGTGGATCAGGTGGAGGTGGATCT
GGTGGAGGTGGATCTGACATTGAGCTCACCCAGTCTCCAAAATTCATGTC
CACATCAGTAGGAGACAGGGTCAGCGTCACCTGCAAGGCCAGTCAGAATG
TGGGTACTAATGTAGCCTGGTATCAACAGAAACCAGGACAATCTCCTAAA
CCACTGATTTACTCGGCAACCTACCGGAACAGTGGAGTCCCTGATCGCTT
CACAGGCAGTGGATCTGGGACAGATTTCACTCTCACCATCACTAACGTGC
AGTCTAAAGACTTGGCAGACTATTTCTGTCAACAATATAACAGGTATCCG
TACACGTCCGGAGGGGGGACCAAGCTGGAGATCAAACGGGCGGCCGCAAT
TGAAGTTATGTATCCTCCTCCTTACCTAGACAATGAGAAGAGCAATGGAA
CCATTATCCATGTGAAAGGGAAACACCTTTGTCCAAGTCCCCTATTTCCC
GGACCTTCTAAGCCCTTTTGGGTGCTGGTGGTGGTTGGTGGAGTCCTGGC
TTGCTATAGCTTGCTAGTAACAGTGGCCTTTATTATTTTCTGGGTGAGGA
GTAAGAGGAGCAGGCTCCTGCACAGTGACTACATGAACATGACTCCCCGC
CGCCCCGGGCCCACCCGCAAGCATTACCAGCCCTATGCCCCACCACGCGA
CTTCGCAGCCTATCGCTCCAGAGTGAAGTTCAGCAGGAGCGCAGACGCCC
CCGCGTACCAGCAGGGCCAGAACCAGCTCTATAACGAGCTCAATCTAGGA
CGAAGAGAGGAGTACGATGTTTTGGACAAGAGACGTGGCCGGGACCCTGA
GATGGGGGGAAAGCCGAGAAGGAAGAACCCTCAGGAAGGCCTGTACAATG
AACTGCAGAAAGATAAGATGGCGGAGGCCTACAGTGAGATTGGGATGAAA
GGCGAGCGCCGGAGGGGCAAGGGGCACGATGGCCTTTACCAGGGTCTCAG
TACAGCCACCAAGGACACCTACGACGCCCTTCACATGCAGGCCCTGCCCC
CTCGC

The presently disclosed subject matter also provides a nucleic acid composition comprising a first nucleic acid sequence encoding an antigen-recognizing receptor that binds to an antigen and a second nucleic acid sequence encoding an exogenous IL-1Ra polypeptide.

### 3. Immunoresponsive Cells

The presently disclosed subject matter provides immunoresponsive cells comprising (a) an antigen-recognizing receptor (e.g., CAR or TCR) that binds to an antigen, and (b) a secretable IL-1Ra polypeptide. In certain embodiments, the secretable IL-1Ra polypeptide is an exogenous IL-1Ra polypeptide. In certain embodiments, the antigen-recognizing receptor is capable of activating the immunoresponsive cell. In certain embodiments, the secretable IL-1Ra polypeptide (e.g., exogenous IL-1Ra polypeptide, such as a nucleic acid encoding an IL-1Ra polypeptide) is capable of promoting an anti-tumor effect of the immunoresponsive cell. The immunoresponsive cells can be transduced with an antigen-recognizing receptor and an exogenous IL-1Ra polypeptide such that the cells co-express the antigen-recognizing receptor and the exogenous IL-1Ra polypeptide.

In certain embodiments, the antigen-recognizing receptor (e.g., a CAR) targets the T-cell receptor a constant (TRAC) locus, and the expression of the antigen-recognizing receptor (e.g., a CAR) and the IL-1R1a is controlled by the native TCR alpha promoter elements, as disclosed in Eyquem J. et al Nature (2017); 543, 113-117, which is incorporated by reference in its entireties.

The presently disclosed subject matter further provides immunoresponsive cells comprising (a) an antigen-recognizing receptor (e.g., a CAR or a TCR) that binds to an antigen, and (b) a modified promoter at an endogenous IL-1Ra gene. In certain embodiments, the modified promoter enhances the gene expression of the endogenous IL-1Ra gene. In certain embodiments, the IL-1Ra coding sequence is provided in cis with the antigen-recognizing receptor (e.g., a CAR) in a bicistronic vector, and thus, both antigen-recognizing receptor (e.g., a CAR) and IL-1Ra are under the transcriptional control of one promoter (e.g., the retroviral SFG vector promoter). In certain embodiments, the endogenous IL-1Ra locus is modified to have induced transcription (e.g. by modifying the promoter or by providing/inducing upstream transcription factors that would result in the endogenous IL-1Ra gene expression).

The presently disclosed subject matter also provides immunoresponsive cells comprising (a) an antigen-recognizing receptor (e.g., CAR or TCR) that binds to an antigen, and (b) a soluble antigen-binding fragment that binds to an IL-1 polypeptide, an IL-1 receptor (IL-1R) polypeptide, or an IL-1 receptor accessory protein polypeptide, wherein binding of the soluble antigen-binding fragment to the IL-1 polypeptide, the IL-1R polypeptide or the IL-1 receptor accessory protein polypeptide is capable of inhibiting IL-1/IL-1R signaling. In certain embodiments, the soluble antigen-binding fragment is a single-chain variable fragment (scFv). In certain embodiments, the soluble antigen-binding fragment is a single-domain antibody (e.g., a VHH antibody). In certain embodiments, the antigen-recognizing receptor is capable of activating the immunoresponsive cell. The immunoresponsive cells can be transduced with the antigen-recognizing receptor and the soluble antigen-binding fragment such that the cells co-express the antigen-recognizing receptor and the soluble antigen-binding fragment. In certain embodiments, the soluble antigen-binding fragment binds to an IL-1 polypeptide (e.g., IL-1 alpha or IL-1 beta) and blocks its binding to IL-1R (e.g., IL-1R1). In certain embodiments, the soluble antigen-binding fragment binds to an IL-1R1 polypeptide. In certain embodiments, the soluble antigen-binding fragment binds to an IL-1R1 polypeptide and inhibits the activation of the IL-1/IL-1R signaling. In certain embodiments, the soluble antigen-binding fragment binds to an IL-1 receptor accessory protein (e.g., IL-1RAP) and inhibits the activation of the IL-1/IL-1R signaling.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. In certain embodiments, the immunoresponsive cell is a T cell. The T cell can be a CD4⁺ T cell or a CD8⁺ T cell. In certain embodiments, the T cell is a CD4⁺θT cell. In certain embodiments, the T cell is a CD8⁺ T cell.

Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

In certain embodiments, the immunoresponsive cells are cells of the myeloid lineage. Non-limiting examples of immunoresponsive cells of the myeloid lineage include macrophages, monocytes, neutrophils, basophils, eosinophils, erythrocytes, dendritic cells, and megakaryocytes or platelets. In certain embodiments, the immunoresponsive cell is macrophage.

The presently disclosed immunoresponsive cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including IL-10 and TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the presently disclosed immunoresponsive cells prevent and/or alleviate and/or treat CRS in a subject who receives an immunotherapy (e.g., CAR-T cell therapy). In certain embodiments, the presently disclosed immunoresponsive cells reduce one or more symptoms of CRS of a subject, e.g., a subject who receives an immunotherapy. In certain embodiments, the immunoresponsive cells reduce the level of one or more cytokine, including, but not limited to, IL-1 alpha, IL-1 beta, IL-6, IL-8, IL-10, TNF-α, IFN-γ, IL-5, IL-2, IL-4, G-CSF, GM-CSF, M-CSF, IL-12, IL-15, and IL-17. In certain embodiments, the one or more cytokine is associated with CRS. In certain embodiments, the one or more cytokine is a pro-pro-inflammatory cytokine.

In certain embodiments, the immunoresponsive cells reduce the level of one or more chemokine, including, but not limited to, CCL2, CCL3, CCL5, and CXCL1.

Interleukin-1 Receptor Antagonist

In certain embodiments, a presently disclosed immunoresponsive cell comprises an exogenous IL-1Ra polypeptide. Interleukin-1 Receptor Antagonist (IL-1Ra) (also known as IL1RN, DIRA, IRAP, IL1F3, IL1RA, MVCD4, IL-1RN, IL-1ra, IL-1ra3, ICIL-1RA; GenBank ID: 3557 (human), 16181 (mouse), 60582 (rat), 281860 (cattle), 100034236 (horse).) is a gene encoding a protein of the interleukin 1 cytokine family, which protein inhibits the activities of interleukin 1 alpha (IL1A) and interleukin 1 beta (IL1B), and modulates a variety of interleukin 1 related immune and inflammatory responses. The protein product of IL-1Ra includes, but is not limited to, NCBI Reference Sequences NP_000568.1, NP_001305843.1, NP_776213.1, NP_776214.1, NP_776215.1, XP_011509423.1 and XP_005263718.1. In certain embodiments, the IL-1Ra polypeptide is anakinra. In certain embodiments, the IL-1Ra polypeptide is a synthetic polypeptide.

In certain embodiments, the term “IL-1Ra” or “IL-1Ra cytokine” refers to the bioactive form of IL-1Ra after secretion from a cell (e.g., a form where the signal peptide is cleaved off). A non-limiting example of human IL-1Ra has the following amino acid sequence set forth in SEQ ID NO: 4, which is provided below.

[SEQ ID NO: 4]
MEICRGLRSHLITLLLFLFHSETICRPSGRKSSKMQAFRIWDVNQKTFYL
RNNQLVAGYLQGPNVNLEEKIDVVPIEPHALFLGIHGGKMCLSCVKSGDE
TRLQLEAVNITDLSENRKQDKRFAFIRSDSGPTTSFESAACPGWFLCTAM
EADQPVSLTNMPDEGVMVTKFYFQEDE

In certain embodiments, a murine IL-1Ra polypeptide comprises or has the amino acid sequence set forth in SEQ ID NO: 21, which is provided below. In certain embodiments, a murine IL-1Ra polypeptide comprises or has an amino acid sequence that is at least about 80%, at least about 85%, at least about 90%, at least about 95%, at least about 99%, or at least about 100% homologous or identical to the sequence set forth in SEQ ID NO: 21. In certain embodiments, the IL-1Ra polypeptide comprises a fragment of the amino acid sequence set forth in SEQ ID NO: 21, and the fragment has at least about 80%, at least about 85%, at least about 90%, at least about 95%, at least about 99%, or at least about 100% activity and/or function of the IL-1Ra polypeptide having the amino acid sequence set forth in SEQ ID NO: 21.

[SEQ ID NO: 21]
MEICWGPYSHLISLLLILLFHSEAACRPSGKRPCKMQAFRIWDTNQKTFY
LRNNQLIAGYLQGPNIKLEEKIDMVPIDLHSVFLGIHGGKLCLSCAKSGD
DIKLQLEEVNITDLSKNKEEDKRFTFIRSEKGPTTSFESAACPGWFLCTT
LEADRPVSLTNTPEEPLIVTKFYFQEDQ

In certain embodiments, a secretable IL-1Ra polypeptide refers to a polypeptide or a protein, the cytokine portion of which has at least about 80%, about 85%, about 90%, about 95%, about 96%, about 97%, about 98%, about 99%, or about 100% homologous to the cytokine portion of the protein product of IL-1Ra (GenBank ID: 3557 (human), 16181 (mouse), 60582 (rat), 281860 (cattle), 100034236 (horse)), or a fragment thereof that has immunostimulatory activity. In certain non-limiting embodiments, the secretable IL-1Ra polypeptide comprises a cytokine portion and a signal peptide, optionally joined by a linker peptide. Non-limiting examples of secretable IL-1Ra polypeptides include NCBI Reference Sequences NP_000568.1, NP_001305843.1, NP_776213.1, NP_776214.1, NP_776215.1, XP_011509423.1 and XP_005263718.1.

In certain non-limiting embodiments, the secretable IL-1Ra polypeptide comprises a signal peptide, for example, an IL-2 signal peptide, a kappa leader sequence, a CD8 leader sequence or a peptide with essentially equivalent activity. In certain embodiments, the secretable IL-1Ra polypeptide comprises an IL-2 signal peptide. In certain embodiments, the IL-2 signal peptide comprises or has the amino acid sequence set forth in SEQ ID NO: 8.

In certain non-limiting embodiments, the immunoresponsive cells comprise and express (is transduced to express) a second antigen-recognizing receptor, which binds to a second antigen that is different than the antigen to which the first antigen-recognizing receptor binds. The second antigen can be a tumor antigen (e.g., any tumor antigens disclosed herein) or a pathogen antigen (e.g., any pathogen antigens disclosed herein).

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells are removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

### 4. Vectors

Genetic modification of an immunoresponsive cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include an antigen-recognizing receptor (e.g., a CAR or a TCR), a retroviral vector is generally employed for transduction, however any other suitable viral vector or non-viral delivery system can be used. The antigen-recognizing receptor and the IL-1Ra polypeptide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by microinjection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### 5. Enhancing Endogenous IL-1Ra Gene Expression

Any targeted genome editing methods can be used to modify the promoter/enhancer region of an IL-1Ra gene locus, and thereby enhancing the endogenous expression of IL-1Ra in an immunoresponsive cell. In certain embodiments, the modification comprises replacement of an endogenous promoter with a constitutive promoter or an inducible promoter, or insertion of a constitutive promoter or inducible promoter to the promoter region of an IL-1Ra gene locus. In certain embodiments, a constitutive promoter is positioned on an IL-1Ra gene locus to drive gene expression of the endogenous IL-1Ra gene. Eligible constitutive promoters include, but are not limited to, a CMV promoter, an EF1a promoter, a SV40 promoter, a PGK1 promoter, a Ubc promoter, a beta-actin promoter, and a CAG promoter. Alternatively or additionally, a conditional or inducable promoter is positioned on an IL-1Ra gene locus to drive gene expression of the endogenous IL-1Ra gene. Non-limiting examples of conditional promoters include a tetracycline response element (TRE) promoter and an estrogen response element (ERE) promoter. In addition, enhancer elements can be placed in regions other than the promoter region.

### 6. Genome Editing Methods

Any targeted genome editing methods can be used to modify the promoter/enhancer region of an IL-1Ra gene locus. In certain embodiments, a CRISPR system is used to modify the promoter/enhancer region of an IL-1Ra gene locus. In certain embodiments, zinc-finger nucleases are used to modify the promoter/enhancer region of an IL-1Ra gene locus. In certain embodiments, a TALEN system is used to modify the promoter/enhancer region of an IL-1Ra gene locus.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Modification can be made anywhere within an IL-1Ra gene locus, or anywhere that can impact gene expression of an IL-1Ra gene. In certain embodiments, the modification occurs upstream of the transcriptional start site of an IL-1Ra gene. In certain embodiments, the modification occurs between the transcriptional start site and the protein coding region of an IL-1Ra gene. In certain embodiments, the modification occurs downstream of the protein coding region of an IL-1Ra gene. In certain embodiments, the modification occurs upstream of the transcriptional start site of an IL-1Ra gene, wherein the modification produces a new transcriptional start site.

### 7. Modification of CD40L

The presently disclosed subject matter also provides immunoresponsive cells comprising a modified/altered CD40L. The modification can be knock-down of CD40L (e.g., reduced expression of CD40L), and/or knock-out of CD40L (e.g., elimination/deletion of CD40L). Non-limiting examples of modifications of CD40L include (a) knockout part of or the entirety of a CD40L gene in the immunoresponsive cells; (b) introduction of mutation(s) within a CD40L gene in the immunoresponsive cells, e.g., frameshift mutations that result in non-functional translated proteins; (c) modification (e.g., disruption) of the promoter and/or enhancer elements that control the expression of a CD40L gene in the immunoresponsive cells; (d) downregulation or disruption of the function of the transcription factors that control CD40L expression (e.g., can be performed in inducible or constitutive fashion), (e) downregulation of CD40L protein by expressing inhibitory ribonucleotides targeting the CD40L in the immunoresponsive cells (e.g., can be performed in inducible or constitutive fashion); and (f) modification of a CD40L gene in the immunoresponsive cells to render it resistant to proteolytic cleavage thereby preventing CD40L protein release in soluble form from the surface of the immunoresponsive cells.

The presently disclosed subject matter also provides immunoresponsive cells comprising a soluble antigen-binding fragment that binds to a CD40L polypeptide. In certain embodiments, binding of the soluble antigen-binding fragment to the CD40L polypeptide is capable of inhibiting CD40/CD40L signaling.

The presently disclosed subject matter further provides immunoresponsive cells comprising a soluble antigen-binding fragment or soluble peptide that antagonistically bind to a CD40 polypeptide, binding of the soluble antigen-binding fragment or soluble peptide to the CD40 polypeptide prevents/inhibits the binding of CD40L to CD40.

Any suitable genetic editing methods and systems can be used to modify CD40L. The genome editing methods disclosed in Sections 4 and 6 can be used to modify CD40L. In certain embodiments, the modification of CD40L comprises modifying the CD40L gene, thereby reducing or eliminating the expression of CD40L. In certain embodiments, a CRISPR system is used to modify a CD40L gene. In certain embodiments, the CRISPR system targets a coding region of a CD40L gene. In certain embodiments, the CRISPR system targets a non-coding region of a CD40L gene. In certain embodiments, the CRISPR system targets exon 1 of a human CD40L gene. In certain embodiments, the CRISPR system comprises a guide RNA (gRNA) that targets the exon 1 of a human CD40L gene. In certain embodiments, the gRNA comprises the nucleotide sequence set forth in SEQ ID NO: 38, SEQ ID NO: 39, and SEQ ID NO: 40, which are provided below.

| | |
|---|---|
| CCAAACUUCUCCCCGAUCUG | [SEQ ID NO: 38] |
| UGUGUAUCUUCAUAGAAGGU | [SEQ ID NO: 39] |
| UCUUCAUAGAAGGUUGGACA | [SEQ ID NO: 40] |

In certain embodiments, the CRISPR system comprises a guide RNA (gRNA) that targets the exon 2 of a human CD40L gene. In certain embodiments, the gRNA comprises the nucleotide sequence set forth in SEQ ID NO: 41, and SEQ ID NO: 42, which are provided below.

| | |
|---|---|
| CAAAAUAGAUAGAAGAUGAA | [SEQ ID NO: 41] |
| ACGAUACAGAGAUGCAACAC | [SEQ ID NO: 42] |

In certain embodiments, a zinc-finger nuclease is used to modify a CD40L gene. In certain embodiments, a TALEN system is used to modify a CD40L gene. The modification can be located in the coding region or the non-coding region (e.g., promoter region, enhancer region, etc.) of a CD40L gene).

In certain embodiments, the modification of CD40L comprises use of an RNAi agent, including, but not limited to, shRNA, siRNA, LNA, dsRNA, and miRNA. In certain embodiments, the RNAi agent comprises an shRNA. In certain embodiments, the RNAi agent (e.g., shRNA) targets one or more isoform of CD40L and thereby reduces or eliminates the expression of CD40L.

In certain embodiments, the RNAi agent (e.g., shRNA) is expressed from the same construct that expresses an antigen-recognizing receptor disclosed herein (e.g., a CAR or a TCR). In certain embodiments, a same promoter drives the expressions of both the RNAi agent (e.g., shRNA) and the antigen-recognized receptor (e.g., a CAR or a TCR). In certain embodiments, the expressions of the shRNA and the antigen-recognized receptor (e.g., a CAR or a TCR) are driven by difference promoters. In certain embodiments, the RNAi agent is capable of reducing the expression (e.g., endogenous expression) of CD40L by about 5%, about 10%, about 20%, about 30%, about 40%, about 50%, about 60%, about 70%, about 80%, about 90%, about 95%, about 100% or any intermediate value or range thereof.

The immunoresponsive cell comprising the modified/altered CD40L can be an immunoresponsive cell disclosed herein, e.g., an immunoresponsive cell comprising an antigen-recognizing receptor (e.g., CAR or TCR) that binds to an antigen and a secretable IL-1Ra polypeptide; or an immunoresponsive cell comprising an antigen-recognizing receptor (e.g., CAR or TCR) that binds to an antigen and a modified promoter at the endogenous CD40L gene. In certain embodiments, the antigen-recognizing receptor (e.g., a CAR) targets the TRAC locus, and the expression of the antigen-recognizing receptor (e.g., a CAR) and the IL-1R1a is controlled by the native TCR alpha promoter elements, as disclosed in Eyquem J. et al Nature (2017); 543, 113-117, which is incorporated by reference in its entireties.

### 8. Polypeptides and Analogs

Also included in the presently disclosed subject matter are a CD19, CD28, CD3ζ, and IL-1Ra polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD28, CD3ζ, and IL-1Ra). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., .beta. or .gamma. amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein (e.g., IL-1Ra). Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 9. Administration

Compositions comprising the presently disclosed immunoresponsive cells or compositions comprising thereof can be provided systemically or directly to a subject for treating and/or preventing a neoplasm, a pathogen infection, or an infectious disease. In certain embodiments, the presently disclosed immunoresponsive cells or compositions comprising thereof are directly injected into an organ of interest (e.g., an organ affected by a neoplasm). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 10. Formulations

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, or between about 10⁶ and about 10⁸, at least about 1×10⁵ of the presently disclosed immunoresponsive cells are administered to a subject. More effective cells may be administered in even smaller numbers. In certain embodiments, at least about 1×10⁵, at least about 2×10⁵, at least about 5×10⁵, at least about 1×10⁶, at least about 1×10⁷, at least about 1×10⁸, about 2×10⁸, about 3×10⁸, about 4×10⁸, or about 5×10⁸ of the presently disclosed immunoresponsive cells are administered to a subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001% to 50% (weight) solution in phosphate buffered saline, and the active ingredient is present in the order of micrograms to milligrams, such as about 0.0001% to about 5 wt %, about 0.0001% to about 1 wt %, about 0.0001% to about 0.05 wt % or about 0.001% to about 20 wt %, about 0.01% to about 10 wt %, or about 0.05% to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 11. Methods of Treatments

The presently disclosed subject matter also provides various methods for treatments. For example, the presently disclosed subject matter provides methods of reducing at least one symptom of cytokine release syndrome (CRS) in a subject, methods of reducing tumor burden in a subject, methods of treating and/or preventing a neoplasm in a subject, methods of lengthening survival of a subject having a neoplasm, and methods of treating and/or preventing a pathogen infection or other infectious disease in a subject (e.g., such as an immunocompromised human subject). In certain embodiments, the level of a cytokine is reduced. In certain embodiments, the cytokine is a pro-inflammatory cytokine. In certain embodiments, the cytokine is selected from the group consisting of IL-1 alpha, IL-1 beta, IL-6, IL-8, IL-10, TNF-α, IFN-γ, IL-5, IL-2, IL-4, G-CSF, GM-CSF, M-CSF, IL-12, IL-15, and IL-17.

In certain embodiments, each of the various methods comprises administering an effective amount of presently disclosed immunoresponsive cells or a composition (e.g., a pharmaceutical composition) comprising thereof.

In certain embodiments, the effective amount is an amount sufficient to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, each of the various methods comprises administering to the subject: (a) an effective amount of immunoresponsive cells or a composition (e.g., a pharmaceutical composition) comprising thereof, wherein the immunoresponsive cell comprises an antigen-recognizing receptor that binds to an antigen; and (b) an antibody that binds to CD40L. In certain embodiments, the antigen-recognizing receptor is a chimeric antigen receptor (CAR). In certain embodiments, the immunoresponsive cell further comprises an exogenous IL-1Ra polypeptide. In certain embodiments, the immunoresponsive cell further comprises a modified promoter at an endogenous IL-1Ra gene locus. In certain embodiments, the antibody is an antagonist antibody. In certain embodiments, the antibody blocks CD40L signaling of an immunoresponsive cell. In certain embodiments, the antibody blocks CD40L signaling of a tumor cell. In certain embodiments, the antibody blocks CD40L signaling of a myeloid cell. In certain embodiments, the antibody is a monoclonal antibody. In certain embodiments, the antibody is a human antibody or a humanized antibody. In certain embodiments, the antibody is a chimeric antibody. In certain embodiments, the antibody is a scFv. In certain embodiments, the antibody is a IgG class antibody.

In certain embodiments, each of the various methods comprises administering to the subject (a) an inhibitor of IL-1 signaling, and (b) an immunoresponsive cell comprising an antigen-recognizing receptor that binds to an antigen. In certain embodiments, the inhibitor of IL-1 signaling is selected from the group consisting of IL-1 blocking agents, IL-1R1 blocking agents, and combinations thereof. As used herein, the term “IL-1 blocking agents” refers to agents that are capable of blocking IL-1 (alpha or beta) from binding to its receptor IL-1R1. As used herein, the term “IL-1R1 blocking agents” refers to agents that are capable of blocking IL-1R1 from binding to IL-1, and agents that are capable of preventing/inhibiting IL-1RAP from forming a functional signaling complex with IL-1R1.

In certain embodiments, IL-1 blocking agents are selected from the group consisting of IL-1Ra polypeptides, antibodies that bind to IL-1α, antibodies that bind to IL-1β, and antibodies that bind to both IL-1α and IL-1β, and combinations thereof. In certain embodiments, the IL-1R1 blocking agents are selected from the group consisting of antibodies that bind to IL-1R1, antibodies that bind to IL-1RAP, IL-1R2 polypeptides, and combinations thereof. In certain embodiments, the IL-1 blocking agent is rilonacept. In certain embodiments, the IL-1 blocking agent is an antibody that binds to IL-1β. In certain embodiments, the IL-1β is canakinumab. In certain embodiments, the IL-1Ra polypeptide is anakinra. In certain embodiments, each of the above-noted antibodies (e.g., antibodies binding to IL-1R1, antibodies binding to IL-1α, antibodies binding to IL-1β, antibodies binding to both IL-1α and IL-1β, antibodies binding to IL-1R1, and antibodies binding to IL-1RAP), is an antagonist antibody. In certain embodiments, each of the above-noted antibodies is a monoclonal antibody. In certain embodiments, each of the above-noted antibodies is a human antibody or a humanized antibody. In certain embodiments, each of the above-noted antibodies is a chimeric antibody. In certain embodiments, each of the above-noted antibodies is a scFv. In certain embodiments, each of the above-noted antibodies is a IgG class antibody.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁵-10¹⁰ (e.g., at least about 1×10⁵, at least about 1×10⁶, e.g., about 10⁹) are typically infused. Upon administration of the presently disclosed cells into the host and subsequent differentiation, T cells are induced that are specifically directed against the specific antigen. The modified cells can be administered by any method known in the art including, but not limited to, intravenous, subcutaneous, intranodal, intratumoral, intrathecal, intrapleural, intraperitoneal and directly to the thymus.

Non-limiting examples of neoplasia include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions. In certain embodiments, the neoplasm is a solid tumor.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasm but have not yet evidenced clinical signs of neoplasm. For instance, women testing positive for a genetic mutation associated with breast cancer, but still of childbearing age, can wish to receive one or more of the immunoresponsive cells described herein in treatment prophylactically to prevent the occurrence of neoplasm until it is suitable to perform preventive surgery.

As a consequence of the surface expression of an antigen-recognizing receptor that binds to a tumor antigen and a secretable IL-1Ra polypeptide (e.g., an exogenous IL-1Ra polypeptide), adoptively transferred immunoresponsive cells (e.g., T cells) are endowed with alleviated CRS. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

In certain non-limiting embodiments, the subject does not receive another therapy for preventing, treating and/or alleviating CRS, e.g. a pharmacological intervention. In certain embodiments, the methods are suitable for treatment of a subject without prior, concurrent, simultaneous, or subsequent treatment with one or more other therapies for preventing, treating and/or alleviating CRS, e.g. a pharmacological intervention. In certain embodiments, the subject does not subsequently or simultaneously receive a therapies for preventing, treating and/or alleviating CRS, or does not go on to do so within a certain period of time, such as about 1 day, about 2 days, about 3 days, about 4 days, about 5 days, about 6 days, about 1 week, about 2 weeks, about 3 weeks, about 4 weeks, about 1 month, 2, months, 3 months, 4 months, 5 months, 6 months, 9 months or 1 year subsequent to the administration of the immunoresponsive cells or composition comprising thereof.

### 12. Kits

The presently disclosed subject matter provides kits for treating and/or preventing a neoplasm or a pathogen infection in a subject. In certain embodiments, the kit comprises an effective amount of the presently disclosed immunoresponsive cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a CAR or a TCR) directed toward an antigen of interest and an isolated nucleic acid molecule encoding an IL-1Ra polypeptide in expressible (and secretable) form, which may optionally be comprised in the same or different vectors.

If desired, the immunoresponsive cells and/or nucleic acid molecules are provided together with instructions for administering the cells or nucleic acid molecules to a subject having or at risk of developing a neoplasm or pathogen or immune disorder. The instructions generally include information about the use of the composition for the treatment and/or prevention of neoplasm or a pathogen infection. In certain embodiments, the instructions include at least one of the following: description of the therapeutic agent; dosage schedule and administration for treatment or prevention of a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 13. Novel Mouse Model for CRS and Method of Making and Use

The presently disclosed subject matter provides novel mouse models that recapitulate clinical features of CRS, which can be used for screening therapeutic agents for preventing, alleviating and/or treating CRS. In certain embodiments, the presently disclosed subject matter provides a mouse comprising (a) a tumor cell and (b) an immunoresponsive cell comprising an antigen-recognizing receptor that binds to an antigen. In certain embodiments, the immunoresponsive cell is allogeneic. In certain embodiments, the immunoresponsive cell is present in an amount sufficient to induce one or more CRS-related symptom

The mouse can be an immunocompetent mouse or an immunodeficient mouse. In certain embodiments, the mouse is an immunodeficient mouse. In certain embodiments, the immunodeficient mouse is a SCID-beige mouse. The tumor cell can be a human tumor cell (e.g., a Raji tumor cell) or a murine tumor cell. In certain embodiments, the tumor cell is a human tumor cell.

In certain embodiments, the mouse comprises at least about 10³, about 10⁴, about 10⁵, about 10⁶, about 10⁷, about 10⁸, about 10⁹, about 10¹⁰ of the immunoresponsive cells. In certain embodiments, the immunoresponsive cell is a T cell. In certain embodiments, the antigen-recognizing receptor comprised in the immunoresponsive cell is a CAR.

The presently disclosed subject matter also provides methods for making such mouse. In certain embodiments, the method comprises introducing a presently disclosed immunoresponsive cell into a mouse comprising a tumor cell. In certain embodiments, the method further comprises introducing the tumor cell to a mouse (e.g., an immunodeficient mouse). In certain embodiments, the method further comprises introducing a presently disclosed immunoresponsive cell into the mouse after detectable tumor growth in the mouse. To allow tumor growth, the mouse can be engrafted with the tumor cells for about one day, about two days, about three days, about four days, about five days, about six days, about one week, about two weeks, about three weeks, about four weeks, about one month, about two months, about three months, about four months, about five months, about one year or more, or any intermediate time period thereof.

In certain embodiments, the mouse exhibits one or more CRS-related symptom, including, but not limited to, elevated level of one or more pro-inflammatory cytokine, rapid weight loss, piloerection, reduced activity, general presentation of malaise, mortality or a combination thereof. In certain embodiments, the one or more symptom is present about 12 hours after the introduction of the immunoresponsive cells to the mouse. In certain embodiments, the one or more pro-inflammatory cytokine is selected from the group consisting of IL-1 alpha, IL-1 beta, IL-6, IL-8, IL-10, TNF-α, and IFN-γ. In certain embodiments, the mouse does not exhibit Graft versus Host Disease (GvHD).

The mouse can be used for screening an agent that is capable of preventing, alleviating and/or treating CRS. The presently disclosed subject matter provides methods for screening an agent that is capable of preventing, alleviating and/or treating CRS. In certain embodiments, the method comprises:

(a) administering a test agent to a mouse disclosed herein, and

(b) measuring one or more CRS-related symptoms in the mouse;

wherein alleviation of one or more CRS-related symptoms indicates that the test agent is likely to be capable of preventing, alleviating and/or treating CRS. Non-limiting examples of alleviation of one or more CRS-related symptoms include decreased level of one or more of pro-inflammatory cytokine, weight gain, reduced and/or eliminated piloerection, reduced and/or eliminated malaise, prolonged survival, and combinations thereof.

The test agent can be administered to the mouse in any suitable ways, including, but not limited to, systemically or locally, via enteral administration or parenteral administration, or topically.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

### INTRODUCTION

Chimeric Antigen Receptor (CAR) Therapy Targeting CD19 is an Effective Treatment for chemorefractory, relapsed B cell malignancies, especially acute lymphoblastic leukemia (ALL)¹. While a majority of patients will achieve a complete response following a single infusion of CD19 CAR T cells^{2, 3}, the broad applicability of this treatment is hampered by the occurrence of severe cytokine release syndrome (CRS), which is characterized by fever, hypotension and respiratory insufficiency associated with elevated serum cytokines including interleukin-6 (IL6)²⁻⁵. Although manageable, severe CRS may result in multi-organ dysfunction and death in the absence of effective therapeutic intervention^{4, 6-9}. CRS usually occurs within days of CAR T cell infusion at the time of peak CAR T cell expansion and, in ALL, is most frequent and more severe in patients with high tumor burden^{2, 3, 5}. A hallmark of CRS is responsiveness to monoclonal antibody-mediated IL-6 receptor blockade, although this intervention is not always successful and may require further treatment with high dose corticosteroids^{4,6-9}. Improved therapeutic and preventive treatments require a better understanding of CRS physiopathology, which has so far remained elusive. A murine model of CRS was provided wherein the CRS that, like the human syndrome, develops within 2-3 days of CAR T cell infusion, may be lethal and is responsive to IL-6 receptor blockade. Its severity was not mediated by donor T cell-derived cytokines but rather by host derived IL-6, interleukin-1 (IL-1) and Nitric Oxide (NO) that are produced by host myeloid cells, especially macrophages.

### Materials and Methods

Cell Culture.

Burkitt Lymphoma Raji cells and NALM-6 pre-B-ALL cells were obtained from ATCC. Raji GFP-FLuc and NALM-6-GFP-FLuc cells were cultured in RPMI (Invitrogen) supplemented with 10% FBS (HyClone), 10 mM HEPES (Invitrogen), L-Glutamine 2 mM (Invitrogen), NEAA 1× (Invitrogen), 0.55 mM mercaptoethanol, (Invitrogen), Penicillin-Streptomycin 50 U/ml (Invitrogen). Raji and NALM-6 cells were routinely tested for mycoplasma and found negative.

T Cells.

Primary human T cells were purified from buffy coats of healthy donors by negative magnetic selection (Pan T Cell Isolation Kit, Miltenyi). Purified T cells were cultured in XVIVO 15 (Lonza) supplemented with 5% Human Serum AB (Gemini), 10 mM HEPES, 2 mM GlutaMax (Invitrogen), 1×MEM Vitamin Solution (Invitrogen), 1 mM Sodium Pyruvate (Invitrogen), Penicillin-Streptomycin 50 U/ml (Invitrogen), 60 U/ml recombinant IL-2.

Mice.

Mice were treated under a protocol approved by the MSKCC Institutional Animal Care and Use Committee. CRS Model: 6-8 week old female C.B.Igh-1b/GbmsTac-Prkdc^{scid}Lyst^{bg}N7 (SCID-beige) mice (Taconic) were intraperitoneally injected with 3 million Raji-GFP-Fluc cells and tumors were left to grow for 20 days. Tumor burden was evaluated by in vivo bioluminescent imaging two days prior to CAR T cell transfer. Outliers, mice with inconsistently higher or lower tumor burdens were excluded from the experiment. Mice were injected intraperitoneally with 30 million CAR⁺ T cells in PBS supplemented with 2% Human Serum. Control mice received PBS supplemented with 2% Human Serum. Stress test model: 6-8 week-old male NOD.Cg-Prkdc^{scid}Il2rg^{tmWjl}/SzJ (NSG) mice (Jackson Laboratory) were inoculated with 0.5×10⁶ NALM-6-GFP-Fluc cells by tail vein injection followed by with 0.2×10⁶ or with 0.5×10⁶ CAR T cells four days later. Bioluminescence imaging utilized the Xenogen IVIS Imaging System (Xenogen) with Living Image software (Xenogen) for acquisition of imaging datasets. Tumor Burden was assessed as previously described³⁴.

Mouse Treatment.

Anakinra was administered intraperitoneally at 30 mg/kg once per day for 5 days, starting 5 hours prior to CAR T cell transfer. Anti-murine IL-6 (clone MP5-20F3, BioXcell) and anti-murine IL-6R (clone 15A7, BioXcell) were administered intraperitoneally once per day at 25 mg/kg for the first dose and 12.5 mg/kg for subsequent doses for 5 days starting 5 hours prior to CAR T cell transfer. L-NIL (Enzo Life Sciences) or 1400W (Cayman Chemical) were administered intraperitoneally at 5 mg/kg once per day for 5 days starting 5 hours prior to CAR T cell transfer.

Flow Cytometry.

Antibodies were titrated for optimal staining. The following fluorophore conjugated antibodies were used (“h” prefix denotes anti-human, “m” prefix denotes anti-mouse): hCD4 BUV395 (clone RPA-T4, BD), hCD8 PE-Cy7 (clone SK1, eBioscience), hCD3 PerCP-efluor710 (clone OKT3, eBioscience), hCD19 BUV737 (clone SJ25C1, BD), hLNGFR BB515 (clone C40-1457, BD), mF4/80 BV421 and BV711 (clone T45-2342, BD), mLy6C Alexa Fluor 647 and BV786 (clone ER-MP20, AbdSerotec and clone HK1.4, BioLegend respectively), mMHCII BB515 (clone 2G9, BD), mCD11c BV650 (clone N418, BioLegend), mLy6G APC-Fire750 (clone 1A8, BioLegend), mSIGLEC-F PE-CF594 (clone E50-2440, BD), mCD40 BV786 (clone 3/23, BD), mCD40L PE (clone MR1, BD), mCD11b BUV395 (clone M1/70, BD), mNOS2 PE-Cy7 (clone CXNFT, eBioscience). For flow cytometry with live cells 7-AAD (BD) was used as a viability dye. For flow cytometry with fixed cells eFluor506 fixable viability dye (eBioscience) was used. Fc receptors were blocked using Fc Receptor Binding Inhibitor Antibody Human (eBioscience) and Fc Block Mouse (Miltenyi). Cells were fixed using the Intracellular Fixation and Permeabilization Buffer Set (eBioscience) according to the manufacturer's instructions. For CAR staining a Alexa Fluor 647 conjugated goat anti-mouse antibody was used (Jackson Immunoresearch). For cell counting, Countbrite beads were used (Invitrogen) according to the manufacturer's instructions.

Retroviral Vector Constructs and Retroviral Production.

The 1928z-LNGFR construct has been previously described³⁵. 1928z-mCD40L and 1928z-mIL1RN were prepared using standard molecular biology techniques. To obtain the 1928z-mCD40L construct, the cDNA for murine CD40L was inserted in the place of LNGFR. To obtain the 1928z-mIL-1Ra construct, the cDNA for murine IL-1Ra was inserted in the place of LNGFR. Plasmids encoding the SFG γ-retroviral (RV) vector³⁶ were prepared as previously described³⁵. VSV-G pseudotyped retroviral supernatants derived from transduced gpg29 fibroblasts (H29) were used to construct stable retroviral-producing RD114 cell lines as previously described37. T cells were activated with CD3/CD28 T cell Activator Dynabeads (Invitrogen) immediately after purification, at a 1:1 bead-to-cell ratio. After 48 hours of bead activation, T cells were transduced with retroviral supernatants by centrifugation on Retronectin (Takara)-coated plates in order to obtain 1928z-LNGFR, 1928z-mCD40L or 1928z-mIL-1Ra CAR T cells. Transduction efficiency was verified three days later by flow cytometry. CAR T cells were injected in mice 7 days after the first T cell activation.

Cytokine Measurements.

Serum/plasma cytokines were measured using Cytometric Bead Arrays (BD) or ELISA kits for mouse IL-1Ra (Thermo-Fisher) mouse SAA3 (Millipore), as per the manufacturer's instructions.

Animal Pathology.

Mice were transferred to the pathology core facility of Memorial Sloan Kettering where they were sacrificed by cardiac puncture. Tissues obtained were fixed in 10% buffered formalin and were further processed for H&E staining and immunohistochemistry.

RNA Extraction and Transcriptome Sequencing.

Cells were sorted directly into 750 ul of Trizol LS (Invitrogen). The volume was adjusted to 1 ml with PBS and extraction was performed according to instructions provided by the manufacturer. After ribogreen quantification and quality control of Agilent BioAnalyzer, total RNA underwent amplification using the SMART-seq V4 (Clonetech) ultra low input RNA kit for sequencing. For 2-10 ng of total RNA, 12 cycles of amplification were performed. For lesser amount (0.13 to2 ng), 13 cycles of amplification were performed. Subsequently, 10 ng of amplified cDNA was used to prepare Illumina hiseq libraries with the Kapa DNA library preparation chemistry (Kapa Biosystems) using 8 cycles of PCR. Samples were barcoded and run on Hiseq 2500 1T, in a 50 bp/50 bp Paired end run, using the TruSeq SBS Kit v3 (Illumina). An average of 38.5 million paired reads were generated per sample and the percent of mRNA bases was over 77% on average.

RNAseq Analysis.

The output FASTQ data files were mapped (2 pass method) to the target genome (MM10 assembly) using the STAR RNA aligner, resolving reads across splice junctions (ENSEMBL assembly). The first mapping pass used a list of known annotated junctions from ENSEMBL. Novel junctions found in the first pass were then added to the known junctions, after which a second mapping pass was performed using the RemoveNoncanoncial flag. After mapping, the output SAM files were post-processed using PICARD tools to add read groups, AddOrReplaceReadGroups, sort the files and covert to BAM format. The expression count matrix for the mapped reads was then computed using HTSeq. Finally, DESeq was used to normalize the full dataset and analyze differential expression between sample groups.

Program Version.

HT SEQ: htseq/HTSeq-0.5.3. PICARD: picard/picard-tools-1.124 R; R/R-3.2.0. STAR: star/STAR-STAR 2.5.0a. SAMTOOLS: samtools/samtools-0.1.19.

### Results

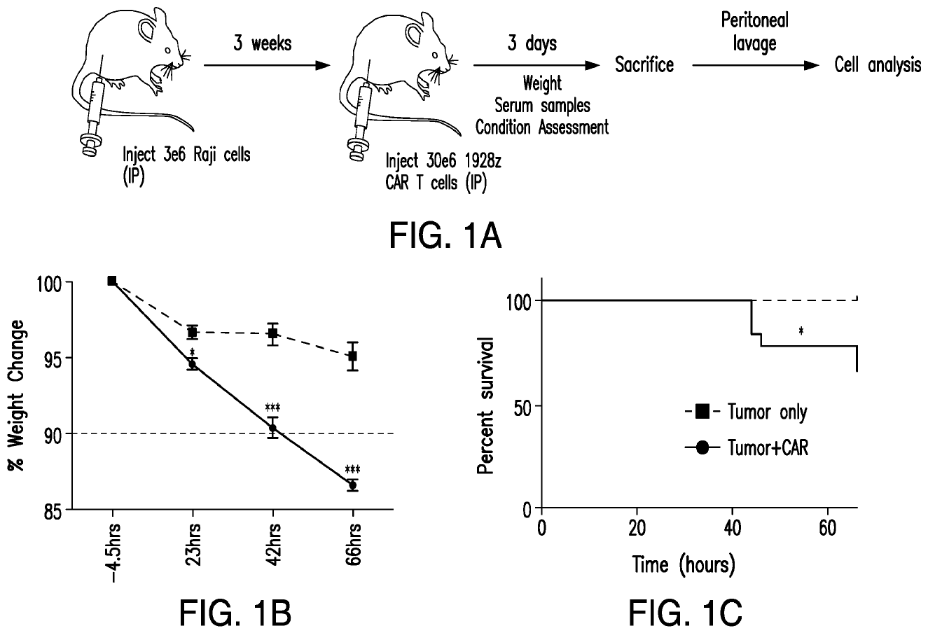

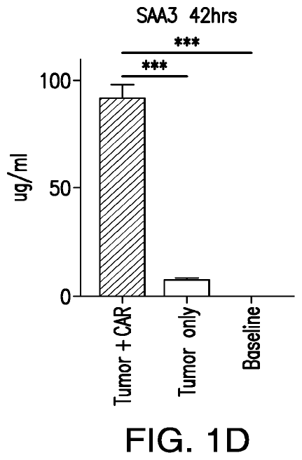

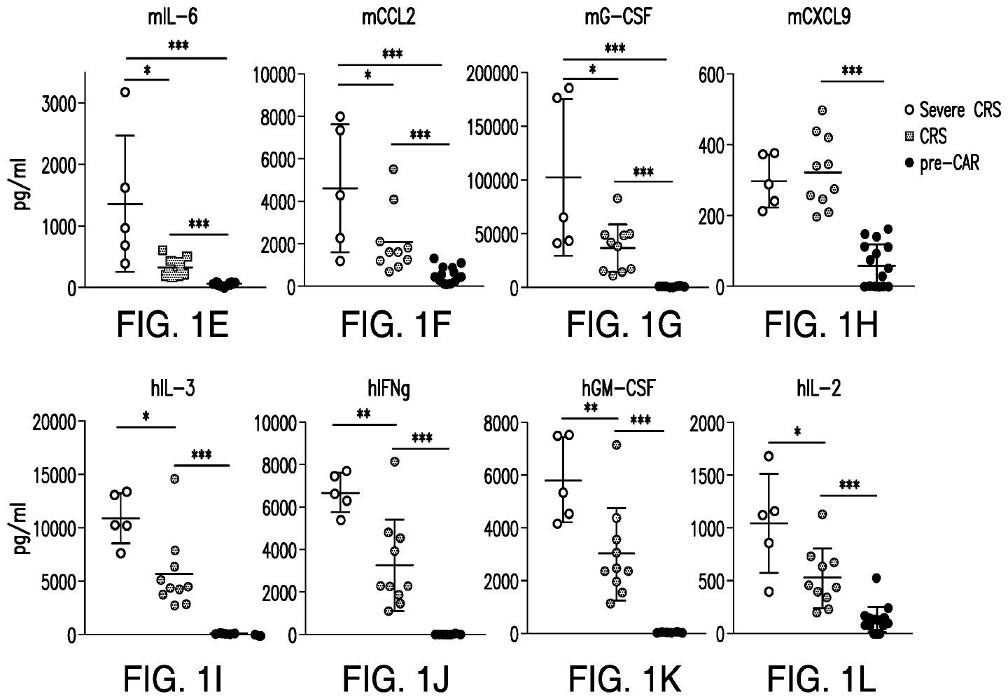

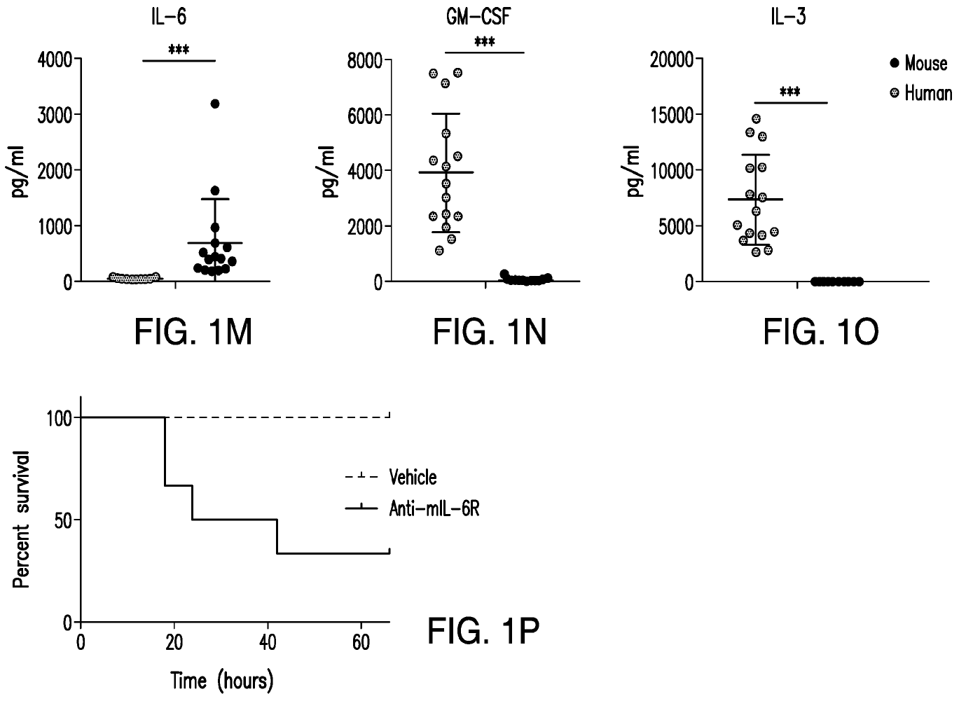

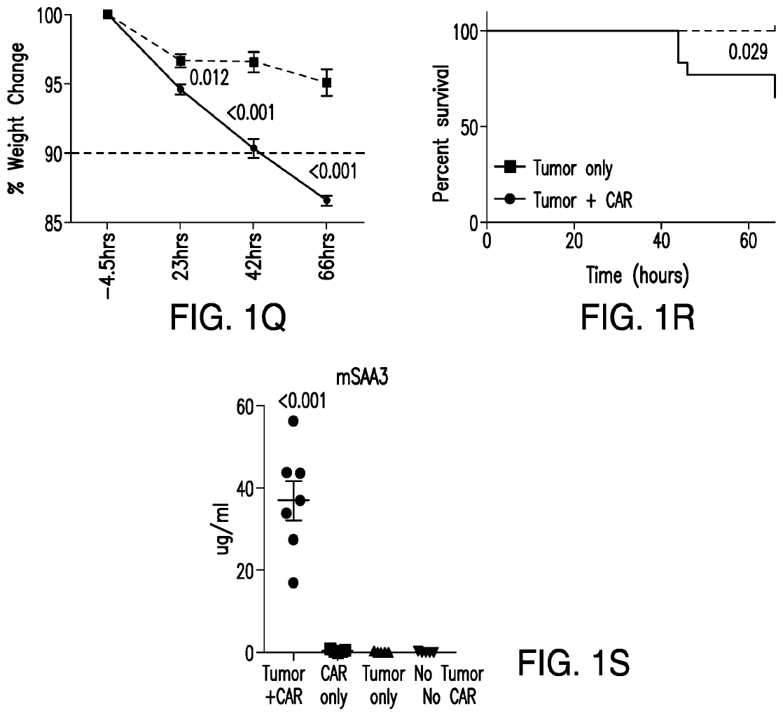

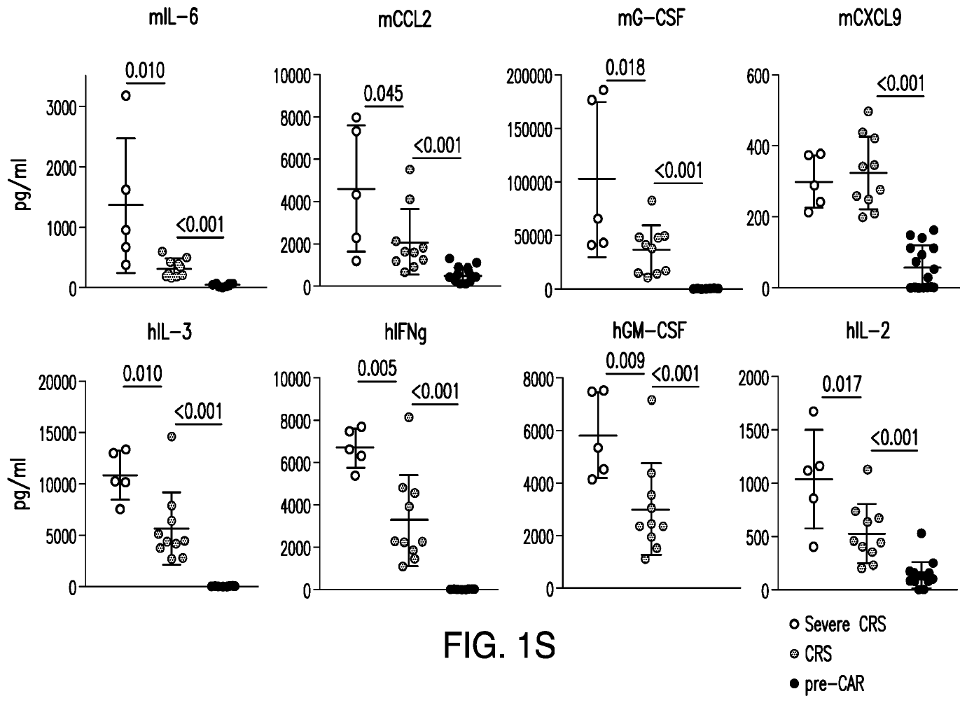

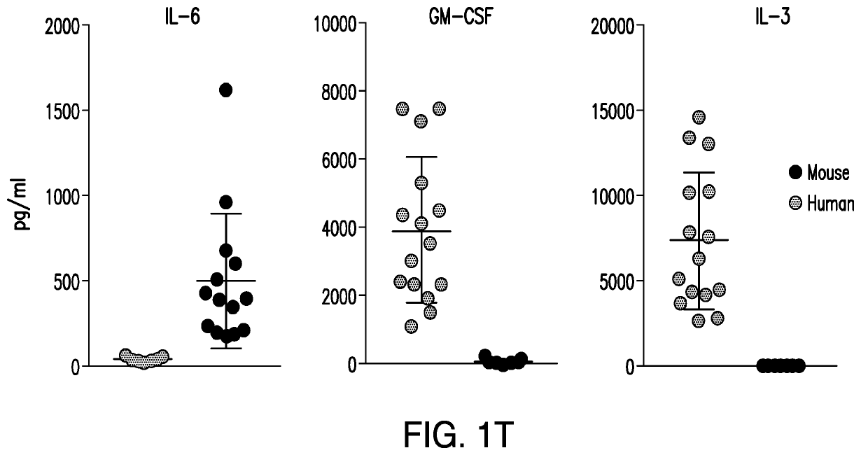

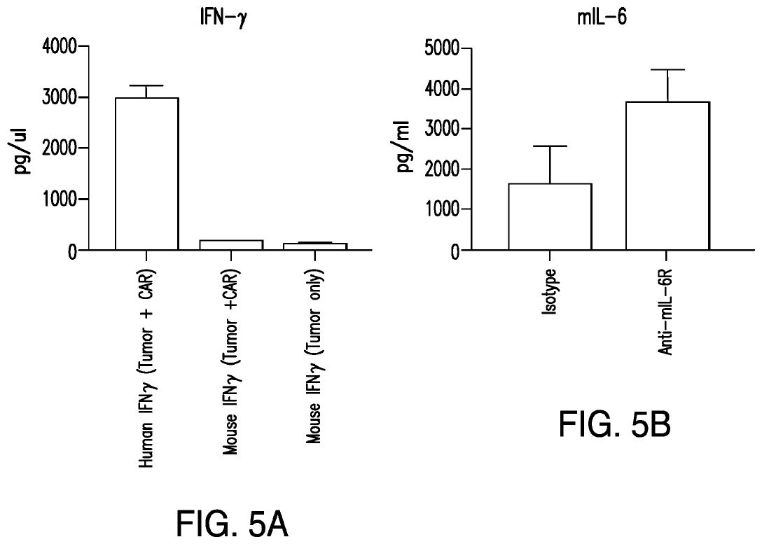

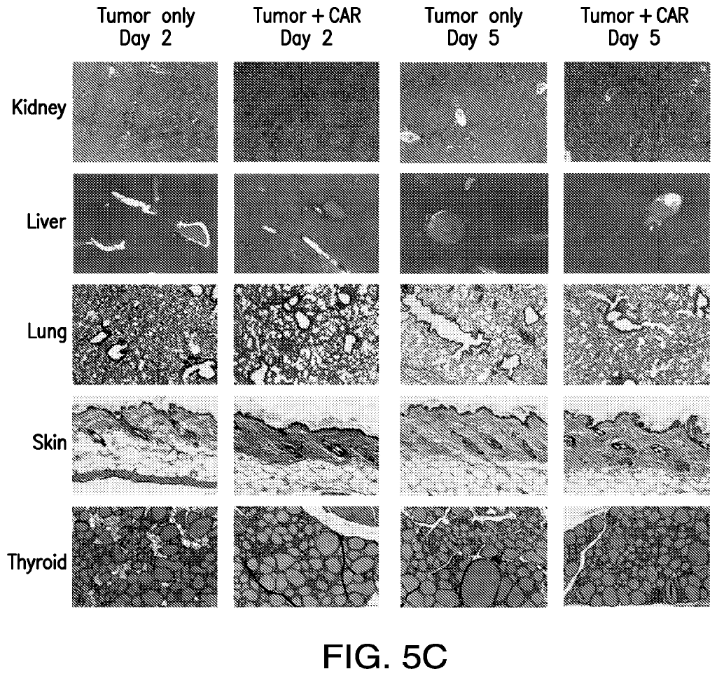

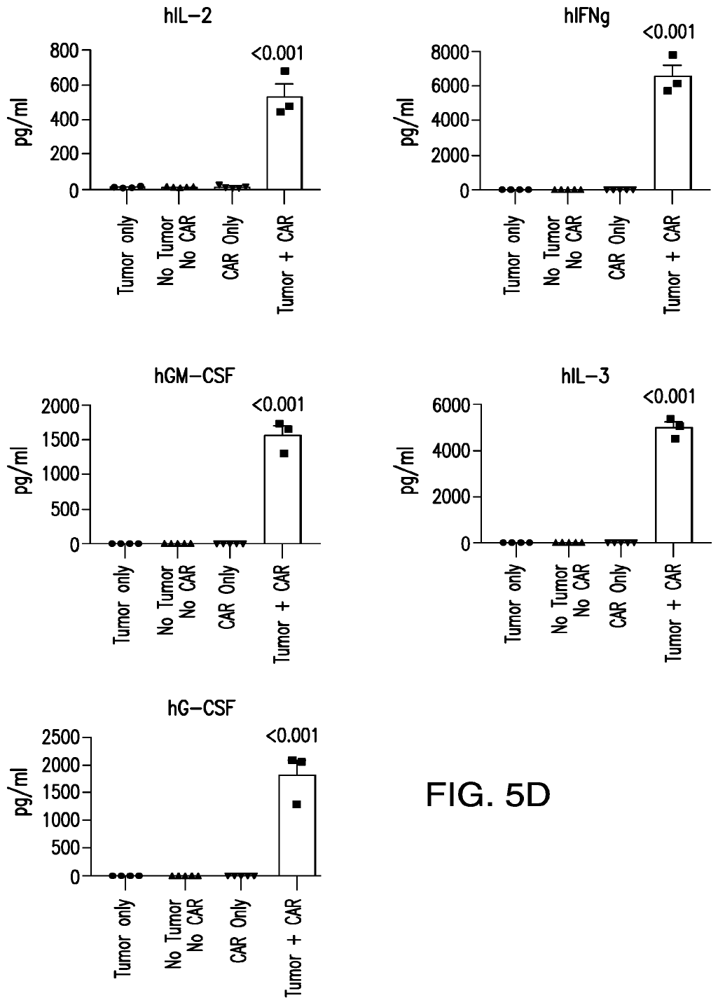

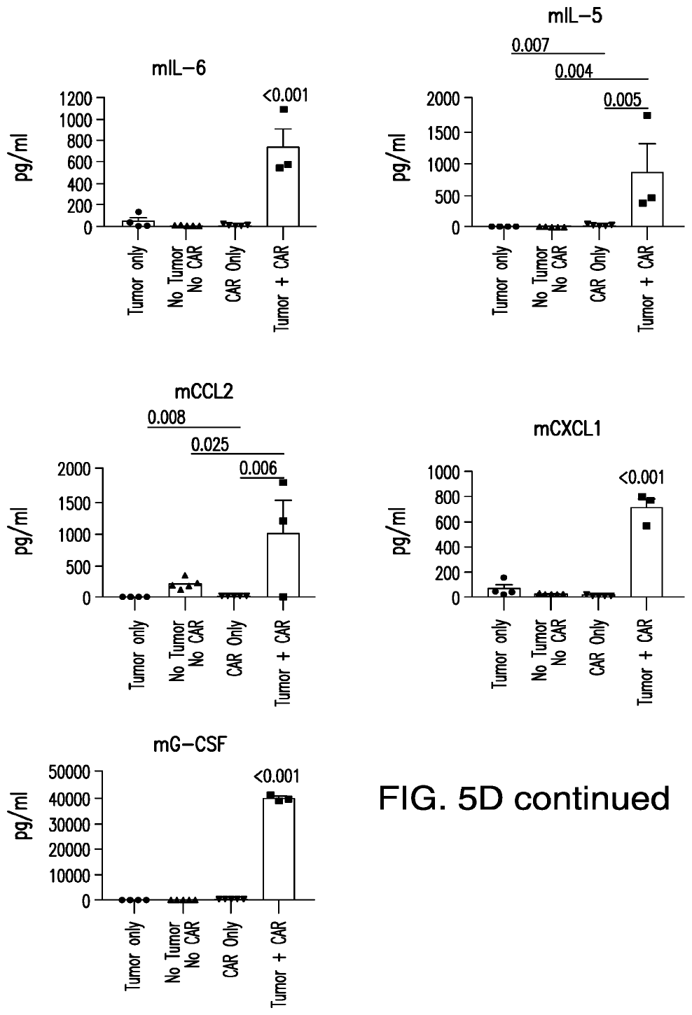

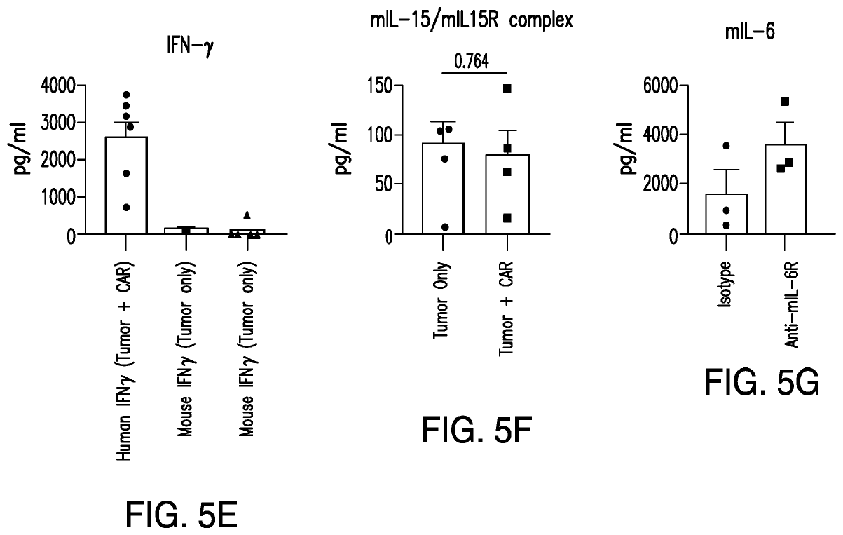

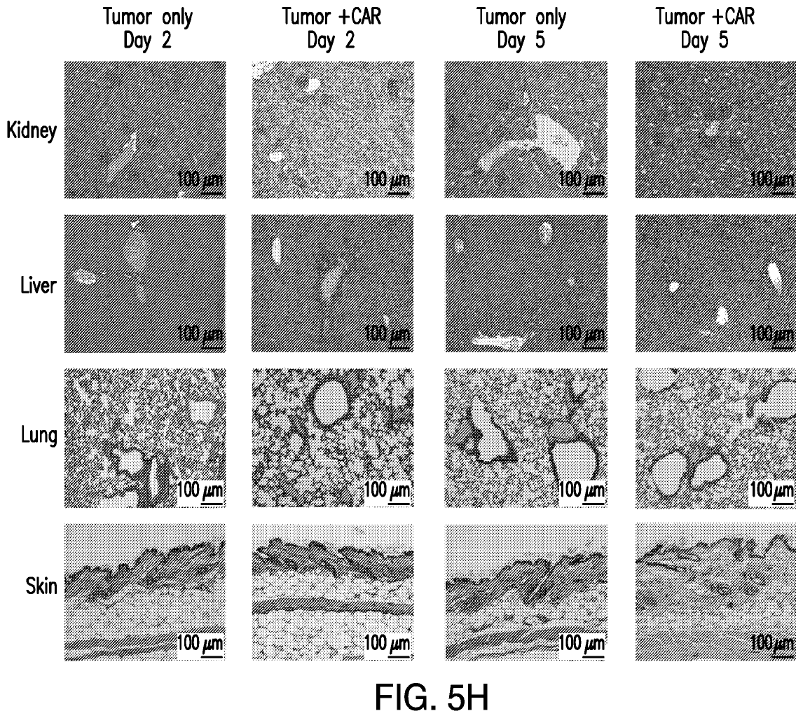

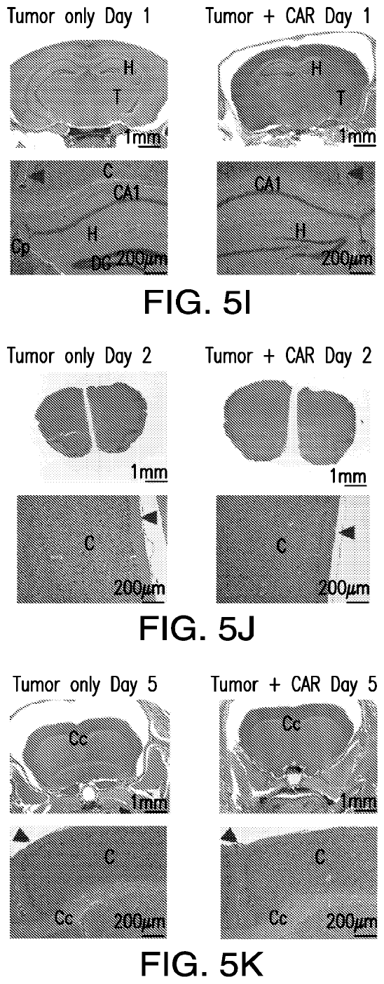

To model CAR T cell-induced CRS, conditions were established whereby a high number of CD19 CAR T cells engaged a high tumor burden and yielded overt toxicity within 2-3 days²⁻⁵ (FIG. 1A). In mice with established intraperitoneal Raji tumors, the administration of 30 million 1928z CAR T cells reproducibly elicited an acute inflammatory response associated with weight loss (FIGS. 1B and 1Q), piloerection, reduced activity, general presentation of malaise and eventual mortality (FIGS. 1C and 1R). Similar to the elevation of C-Reactive Protein (CRP) observed in the clinic^{2, 3, 5} the murine equivalent SAA3^{10, 11} was significantly elevated (FIG. 1D), as were pro-inflammatory cytokines and chemokines including IL-6 (FIG. 1E). The overall levels of these cytokines, including mIL6, mCCL2, mG-CSF, hIL-3, hIFN-γ, hGM-CSF, hIL-2 correlated strongly with CRS severity and survival (FIGS. 1E-1L and 1S). The xenogeneic nature of this model was taken advantage of to discern the origin of these cytokines and chemokines. Thus, IL-6 was produced by endogenous murine cells while IFN-γ and GM-CSF were products of the CART cells. mIL6 and hIL6.¹², were not elevated in the absence of CART cells (FIGS. 1M-1O, 1T and 5D), establishing that this cardinal feature of CRS was the result a multicellular interaction and not the outcome of a T cell-tumor cell interaction. Furthermore, the lack of activity of human IFN-γ¹³ and GM-CSF¹⁴ on the murine receptor suggested that these CAR T cell-derived cytokines were not required for CRS in this model (FIGS. 1K, 5A and 5E), although they could still contribute to it in an autologous setting. In accordance with clinical experience²⁻⁵, treating mice with a murine IL-6R blocking antibody prevented CRS-associated mortality (FIGS. 1P, 5B and 5G). Histopathological analyses performed 2 and 5 days following CAR T cell infusion did not reveal any evidence of Graft-versus-HostDisease (GVHD) (FIGS. 5C and 5H), or evidence of neurotoxicity (FIGS. 5I-5K) consistent with the longer time that would be required to develop GVHD and further supporting that this inflammatory response was initiated by engagement of the CAR on tumor cells.

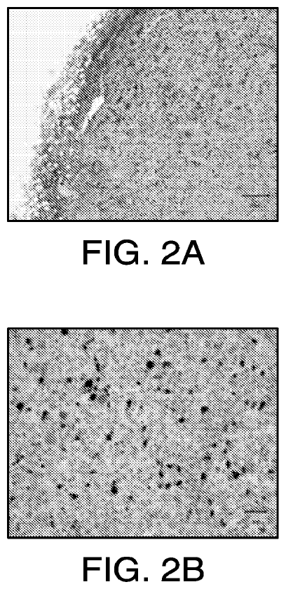

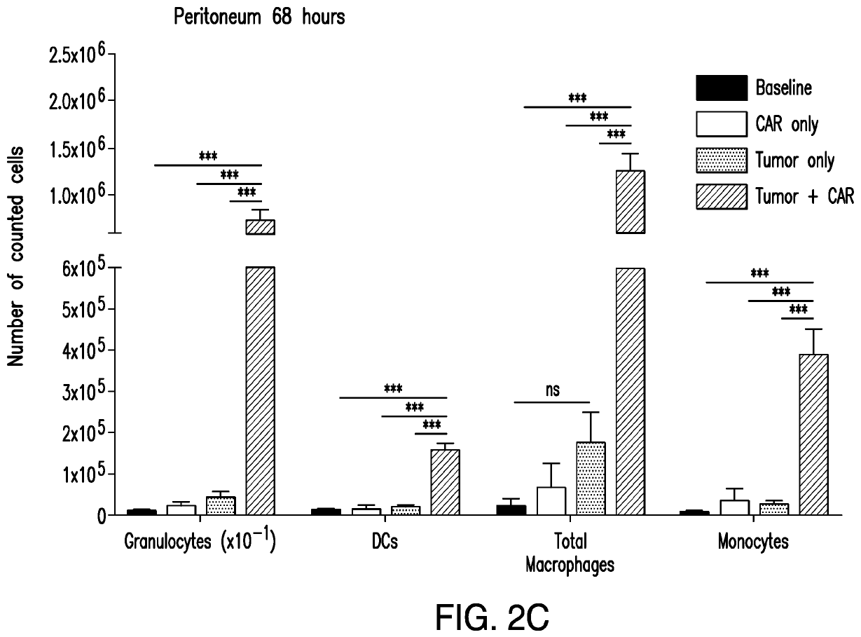

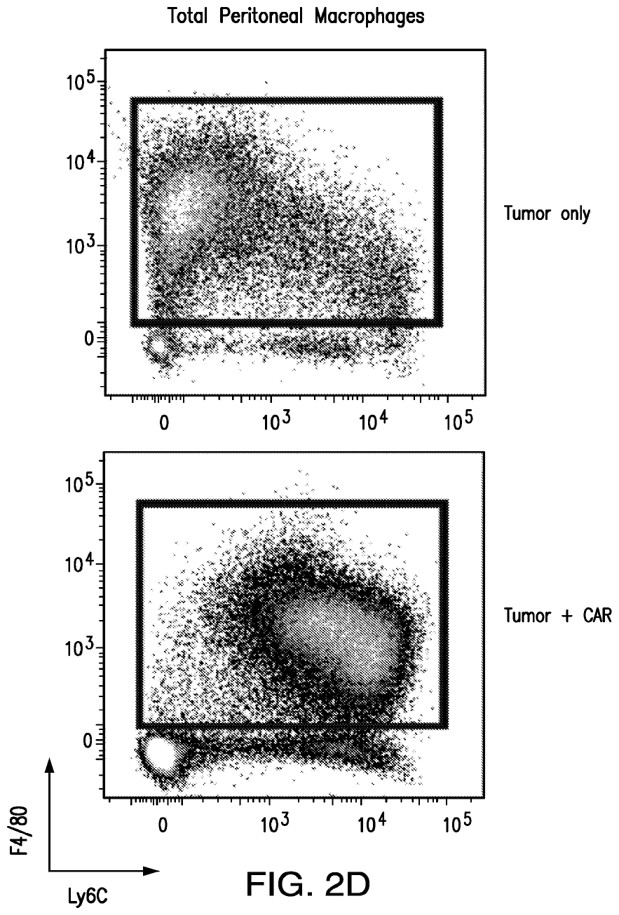

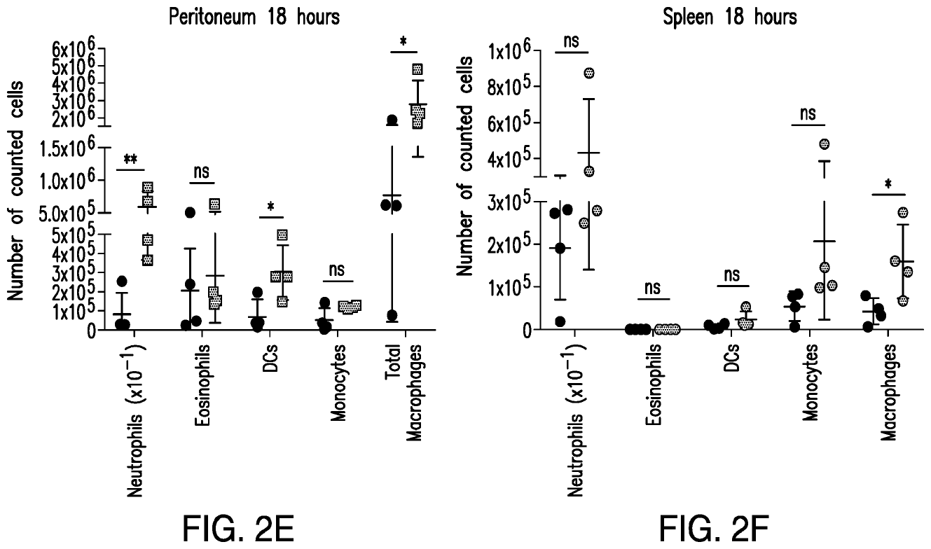

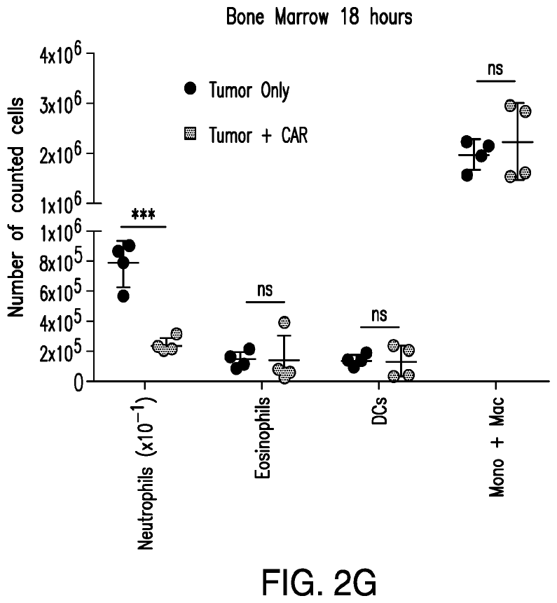

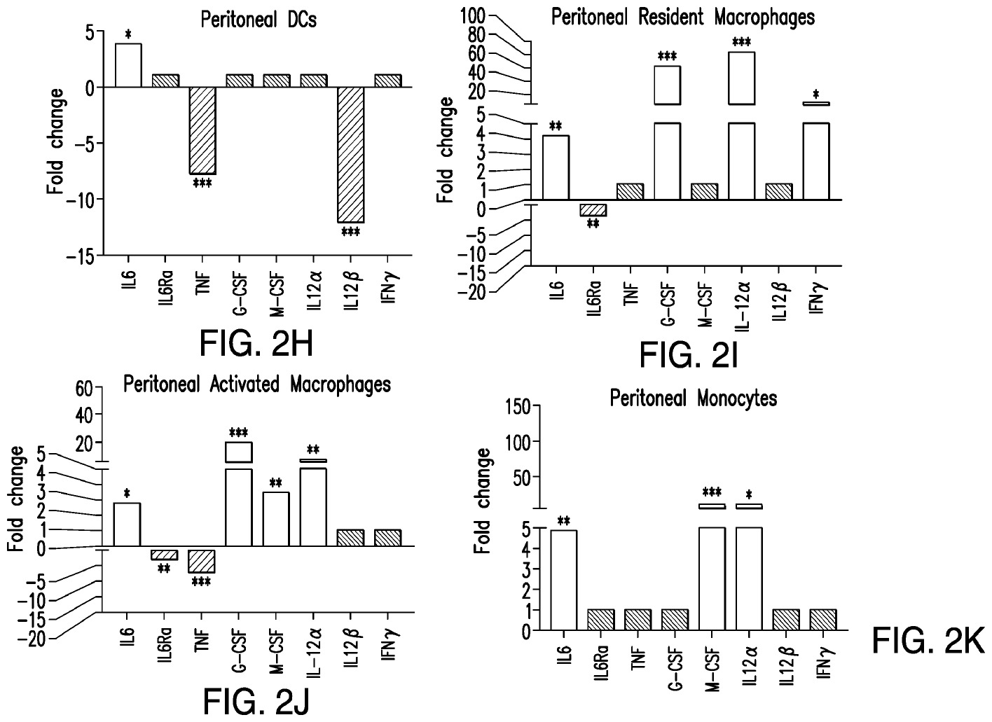

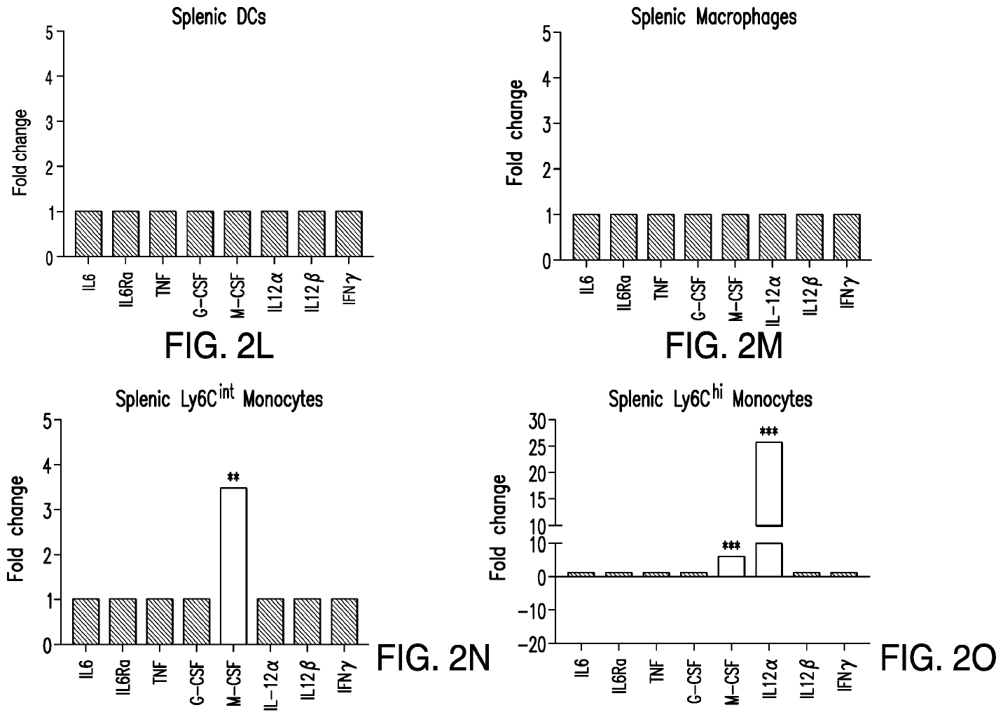

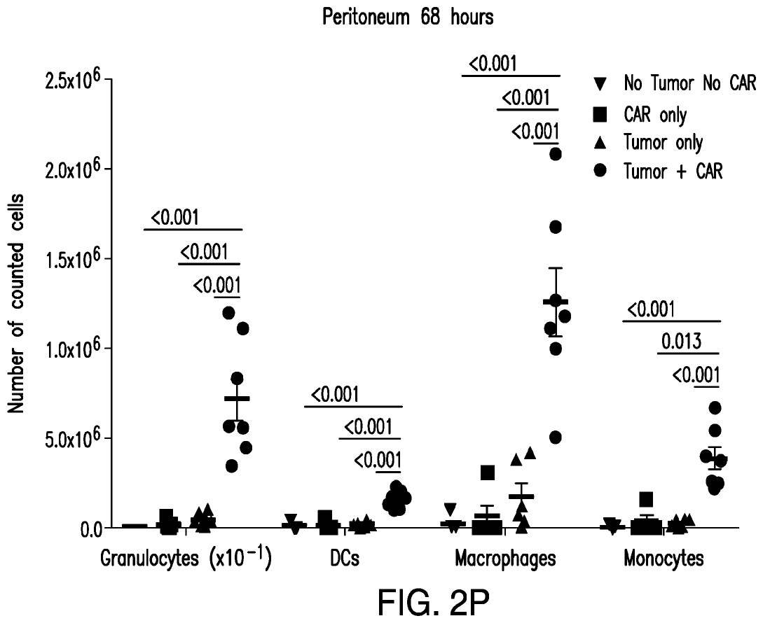

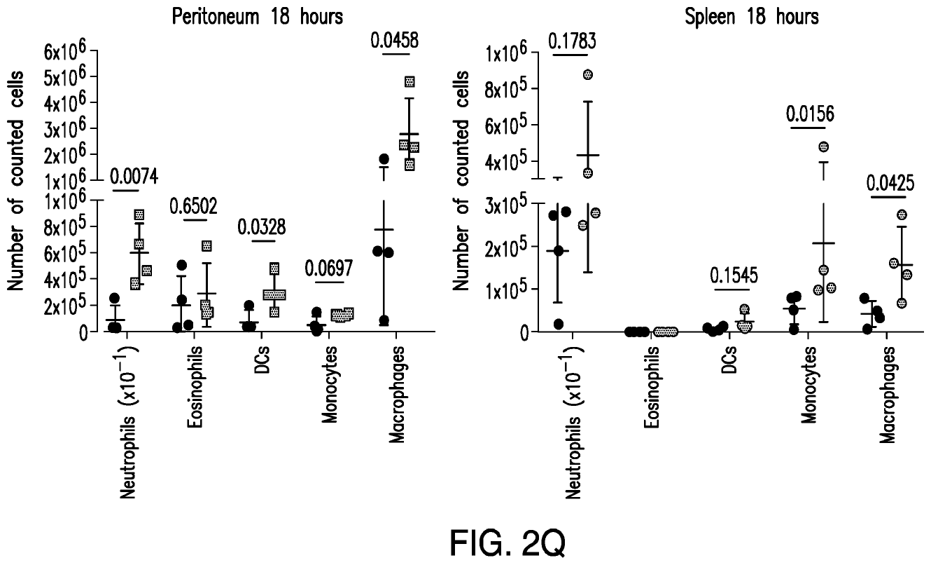

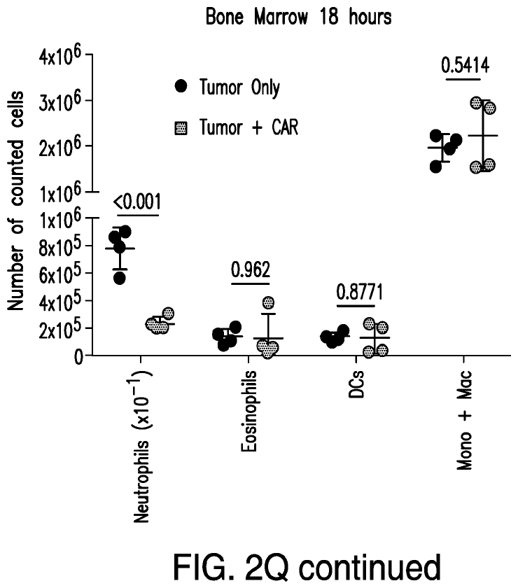

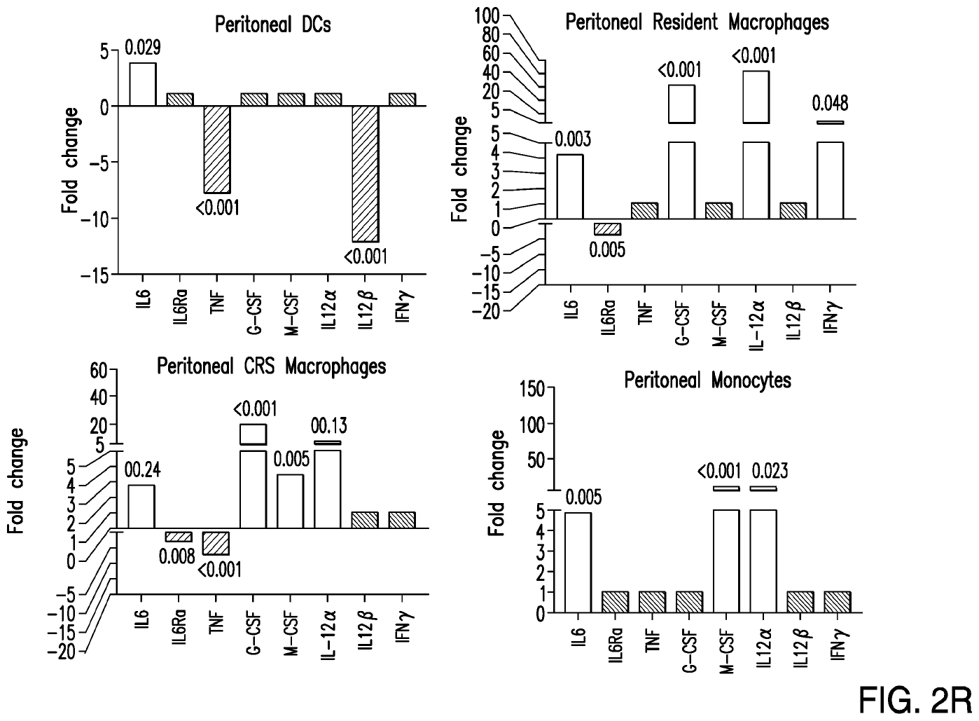

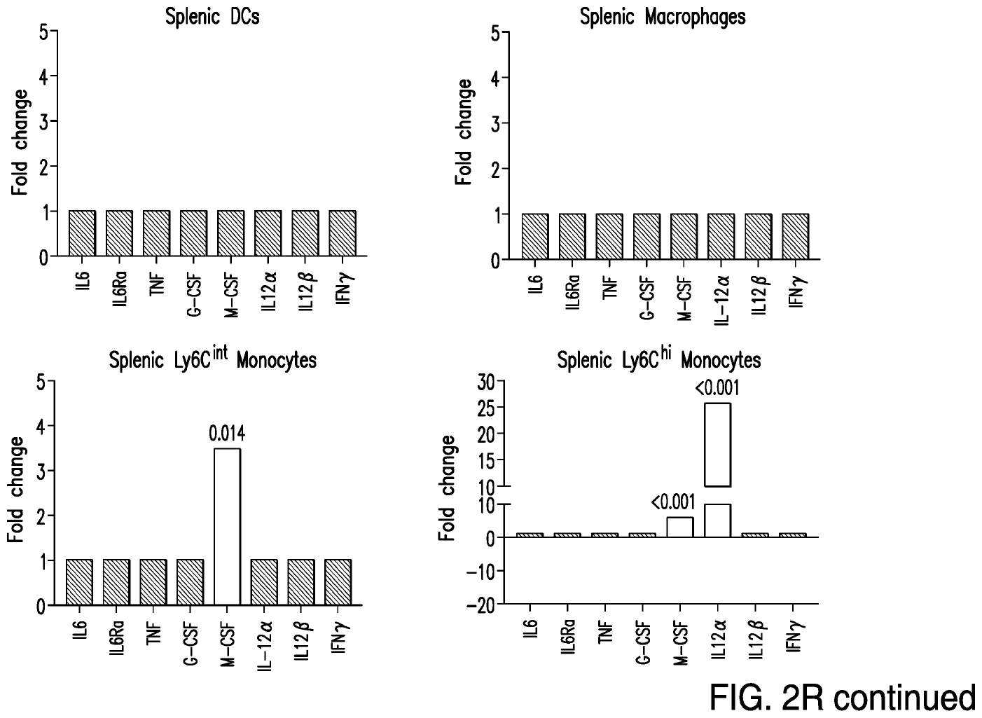

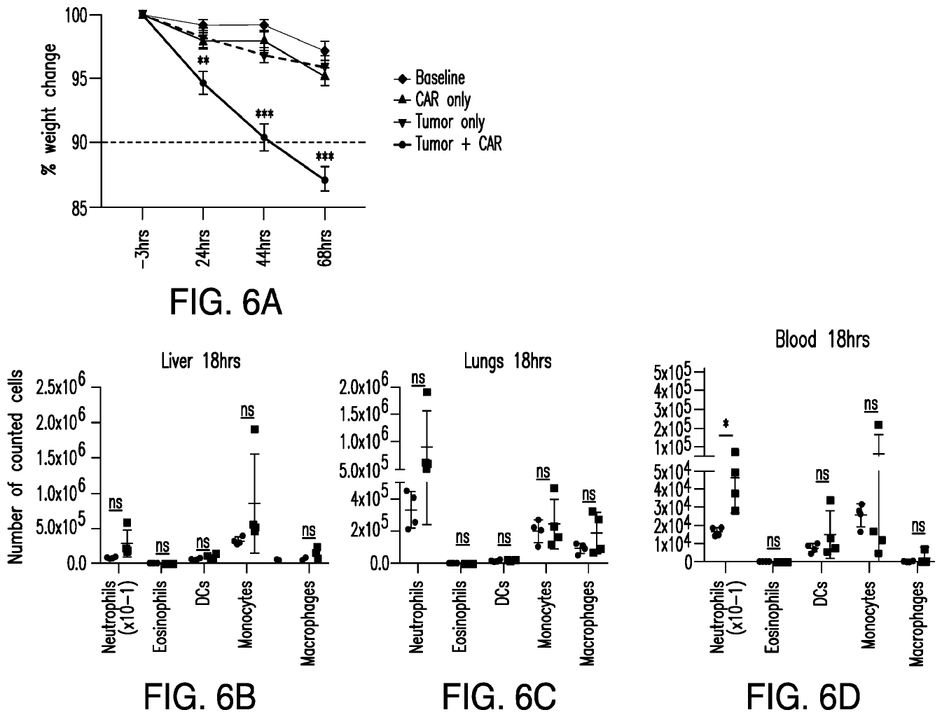

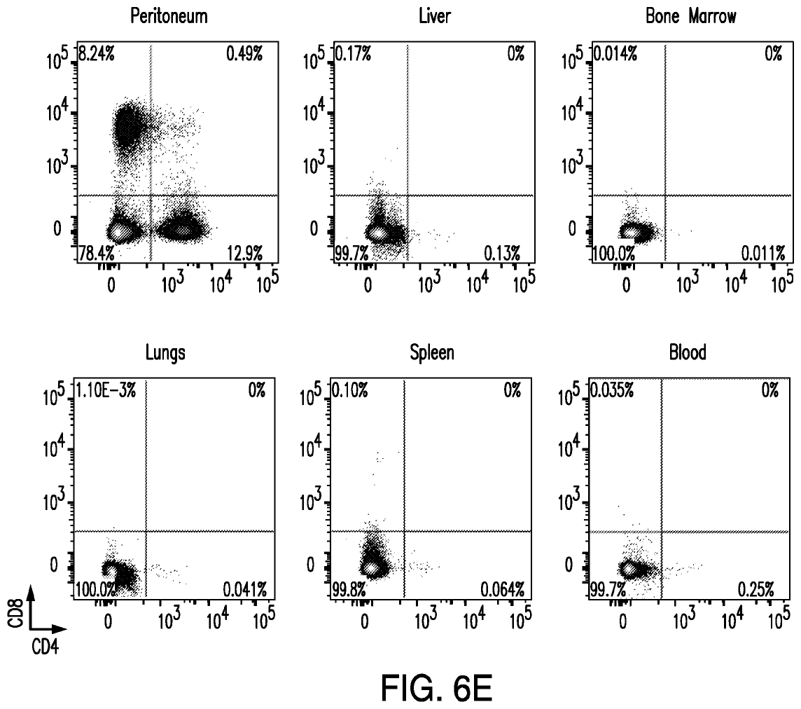

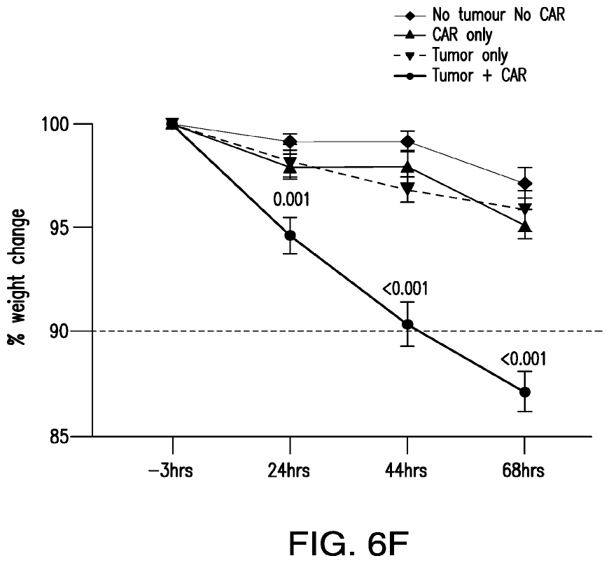

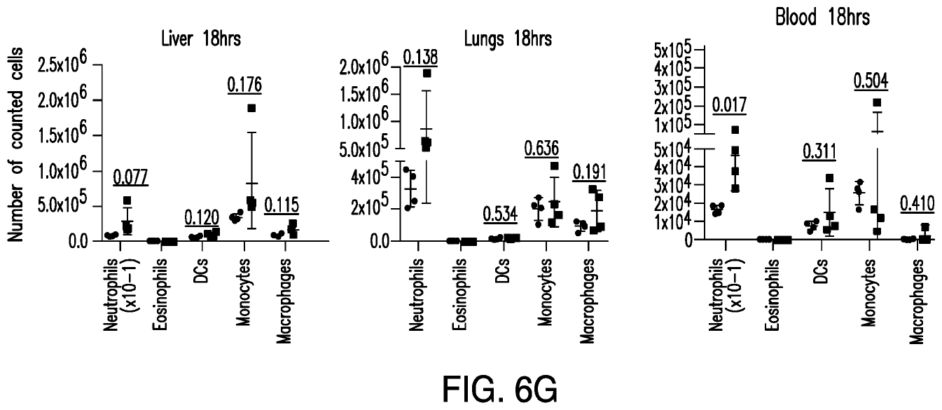

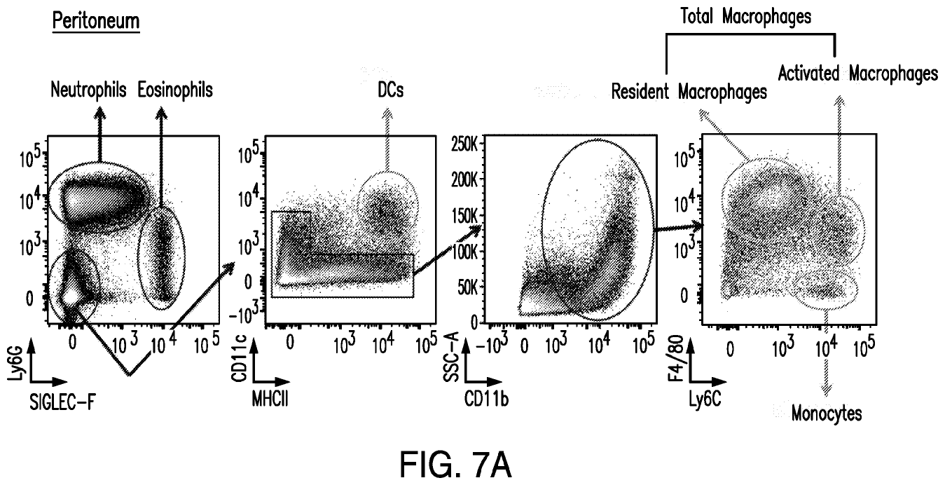

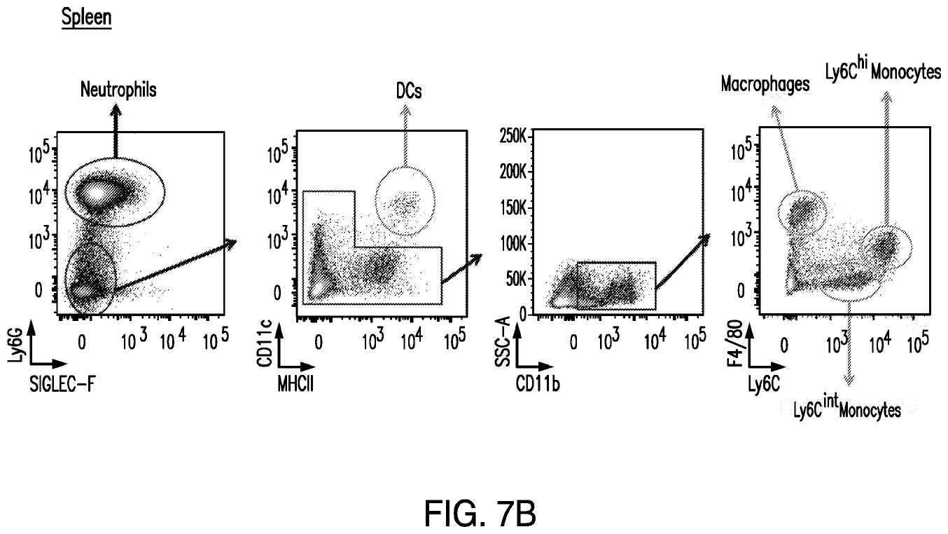

The high serum levels of murine IL6, a predominantly myeloid-derived cytokine together with the presence of tumor-infiltrating myeloid cells (FIGS. 2A and 2B) prior to CAR T cell transfer, led to the hypothesis that myeloid cells would be intimately involved with the induction of CRS. Only after infusion of CAR T cells in the presence of tumor was toxicity observed (FIGS. 6A and 6F) and were myeloid cells found in greater abundance in the peritoneum, (FIGS. 2C and 2P), including neutrophils, eosinophils, dendritic cells (DCs), monocytes, macrophages, and activated macrophages (FIGS. 2D and 7A). The rapid elevation of myeloid cell numbers in mouse peritonea, already noticeable 18 hours after CAR T cell administration (FIG. 2E) suggested that recruitment was a major contributor to this rapid accumulation. To address whether these alterations were regional or systemic, neutrophils, eosinophils, DCs, monocytes and macrophages in other organs (spleen, bone marrow, lungs, liver, peripheral blood) were enumerated. Whereas neutrophils, DCs and macrophages accumulated in the peritoneum, other perturbations were limited to an elevation of macrophage counts in the spleen and neutrophils in peripheral blood, coinciding with neutrophil depletion in bone marrow. Thus, the gross changes in the myeloid compartment were confined to the tumor site and the spleen (FIGS. 2E-2G, 2Q, 6B-6D and 6G). Since IL-6 is a signature cytokine of CRS, it was hypothesized that the presence of IL-6 producing cells would identify the main physiopathological sites. Dendritic cell (DC), macrophage and monocytic populations were therefore purified from peritoneum and spleen (FIGS. 7A and 7B) and RNAseq analysis were performed (neutrophils do not typically produce IL-6¹⁵,). Remarkably, only peritoneal but not splenic DCs, monocytes and macrophages showed upregulated IL-6 transcripts (FIGS. 2H-2O and 2R). As CAR T cells were only found in the peritoneum (FIG. 6E), these findings suggested that IL-6 induction requires proximity of CAR T cells and myeloid cells.

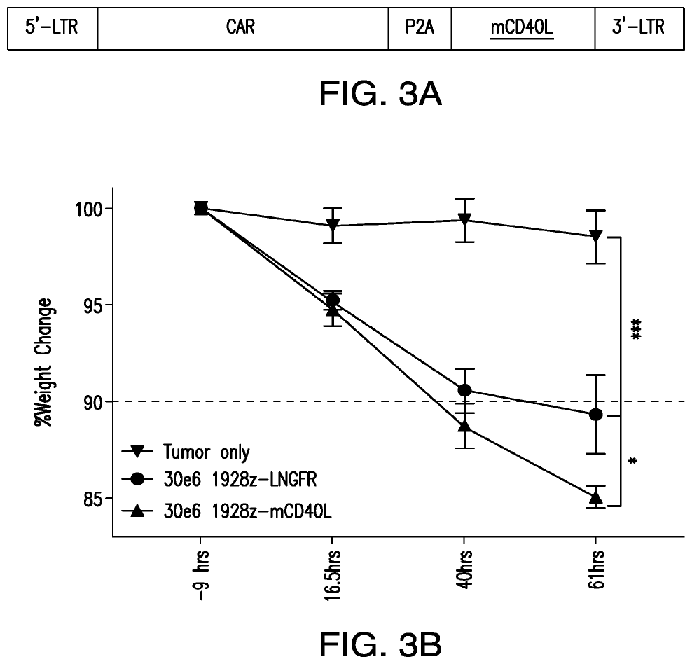

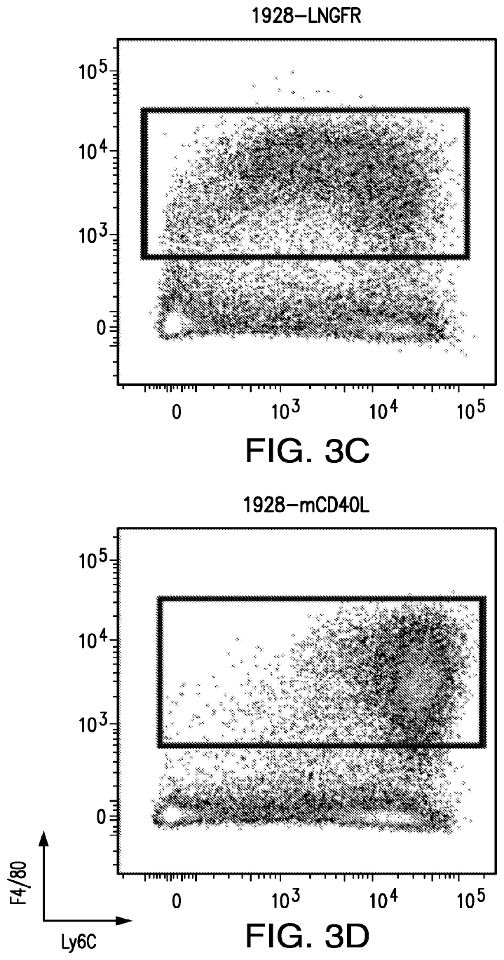

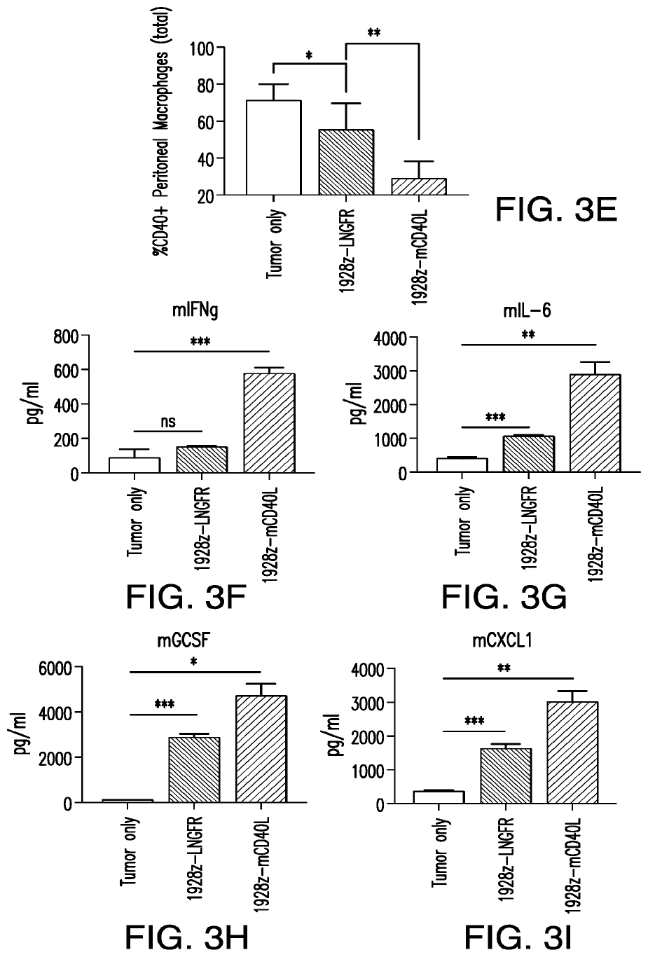

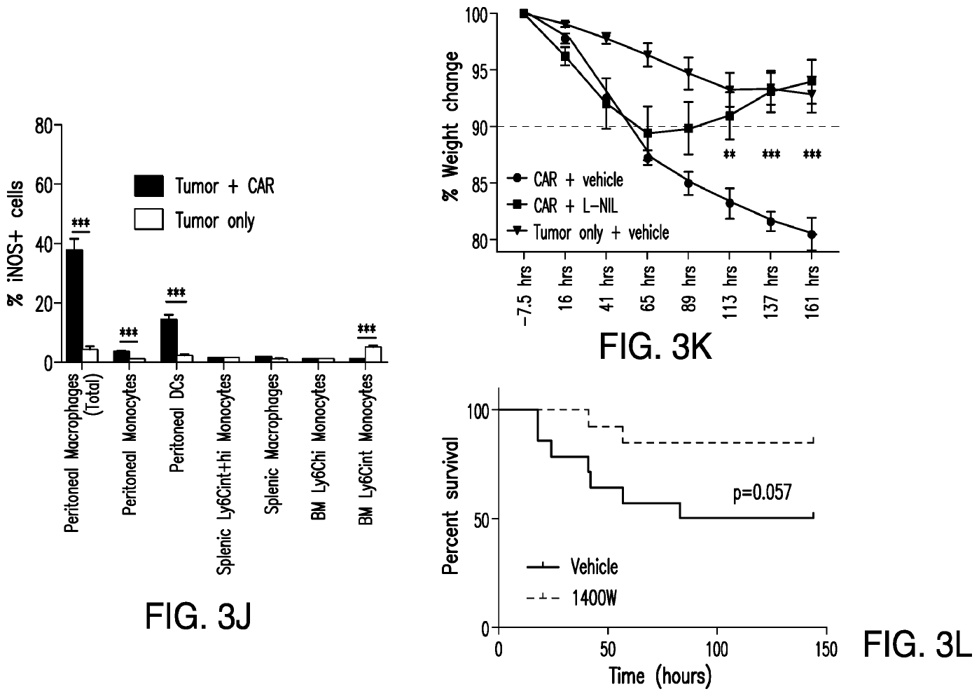

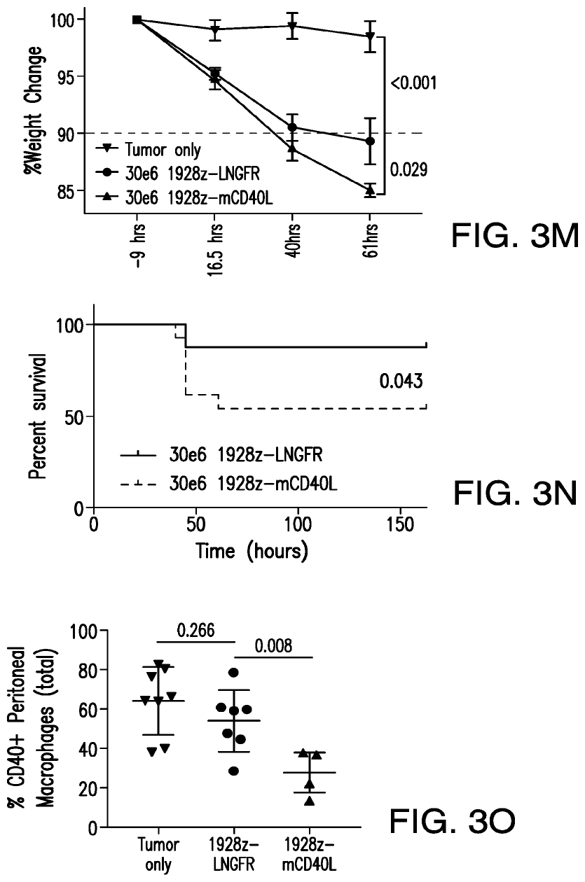

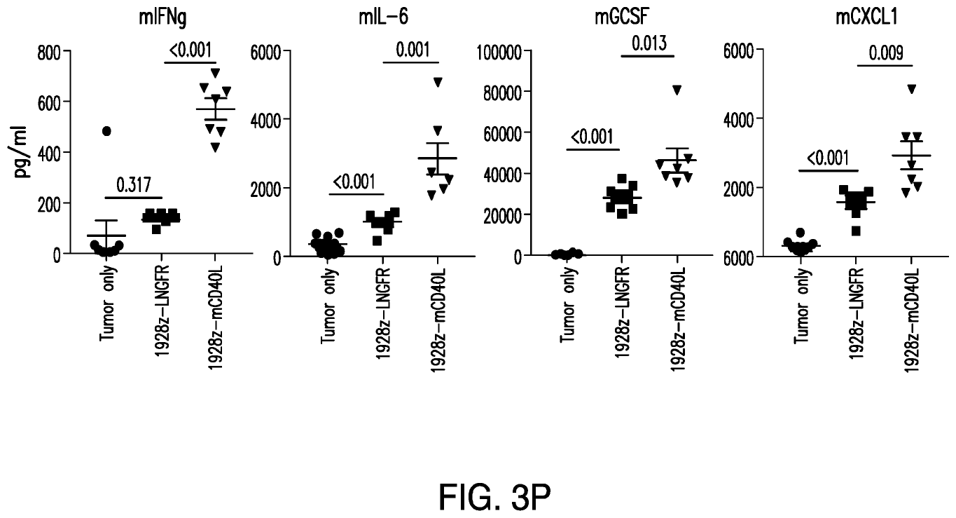

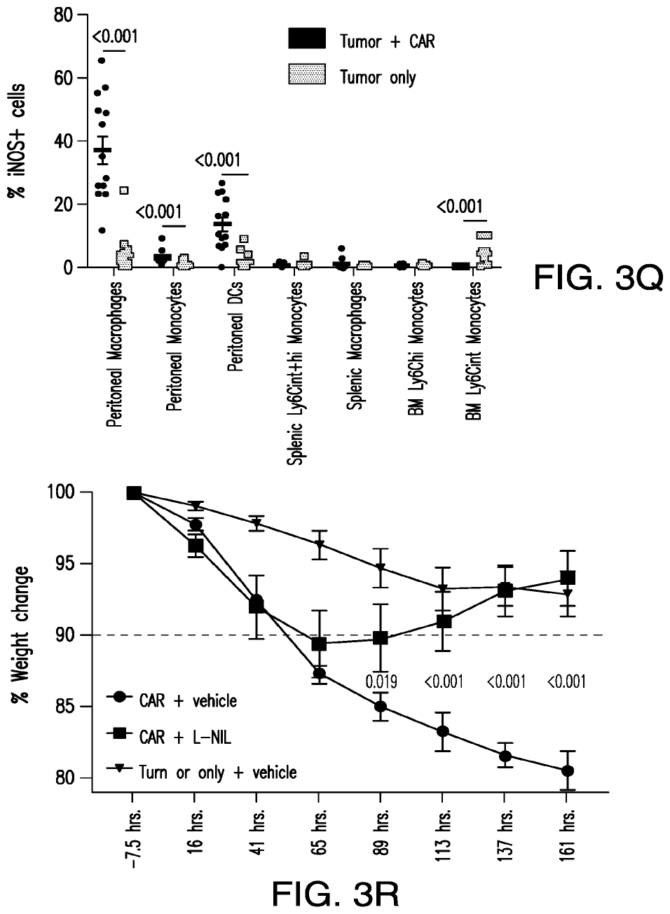

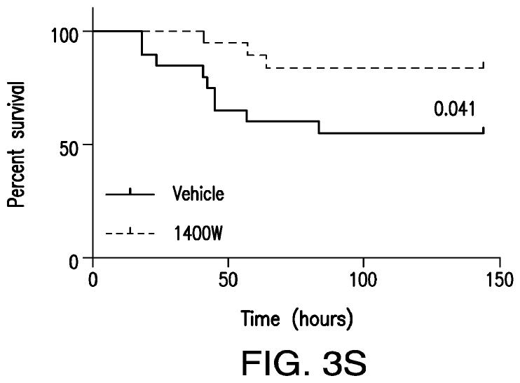

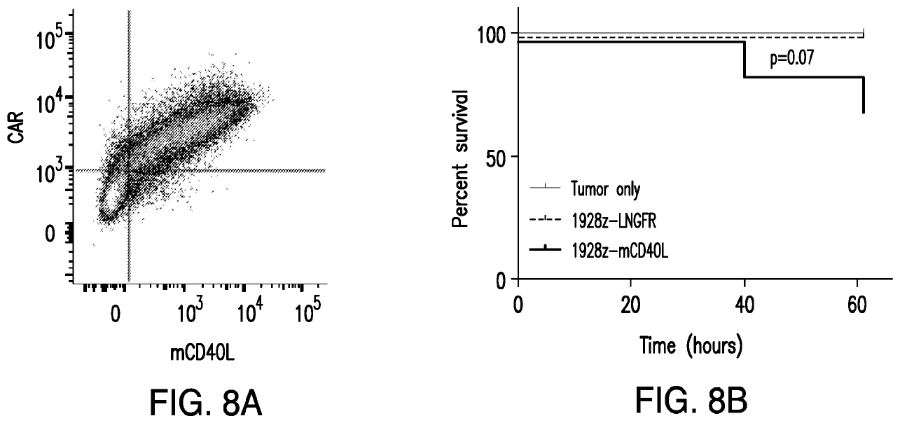

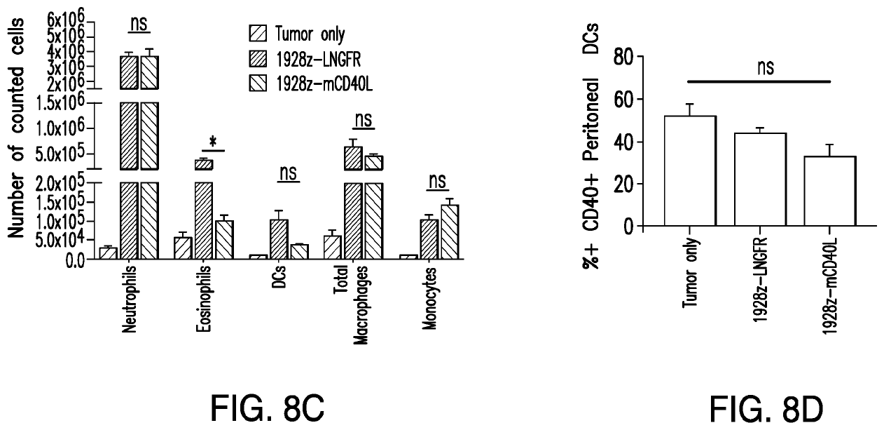

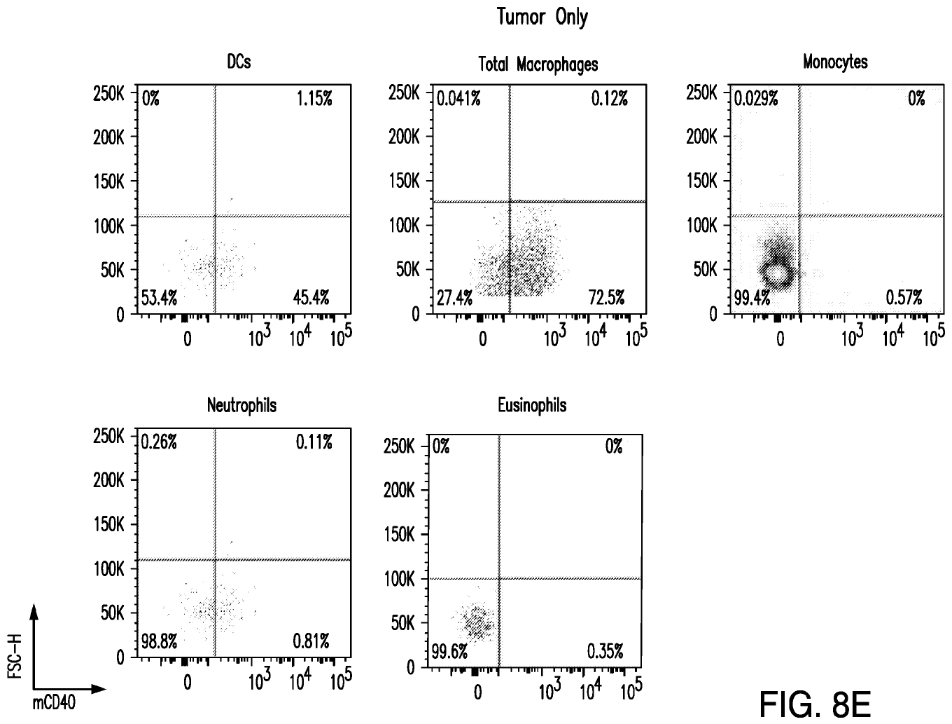

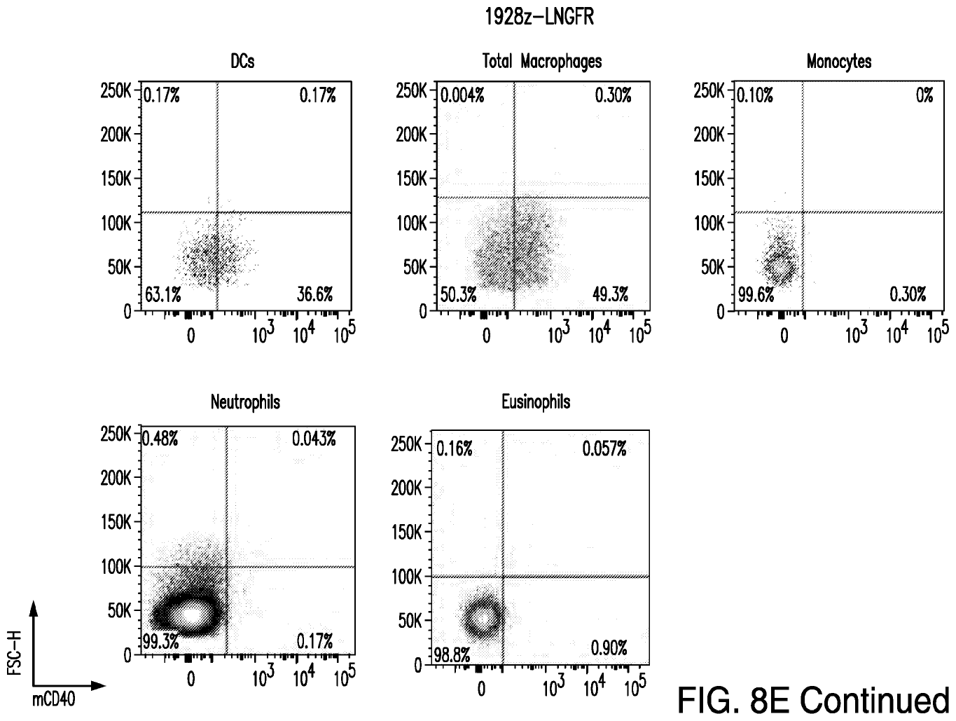

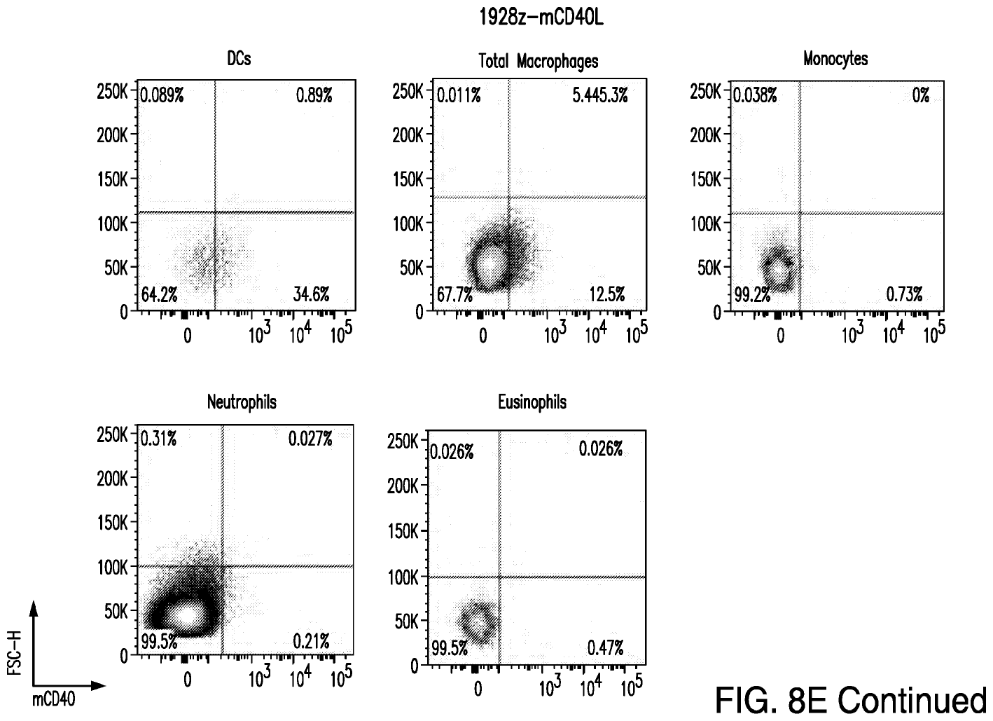

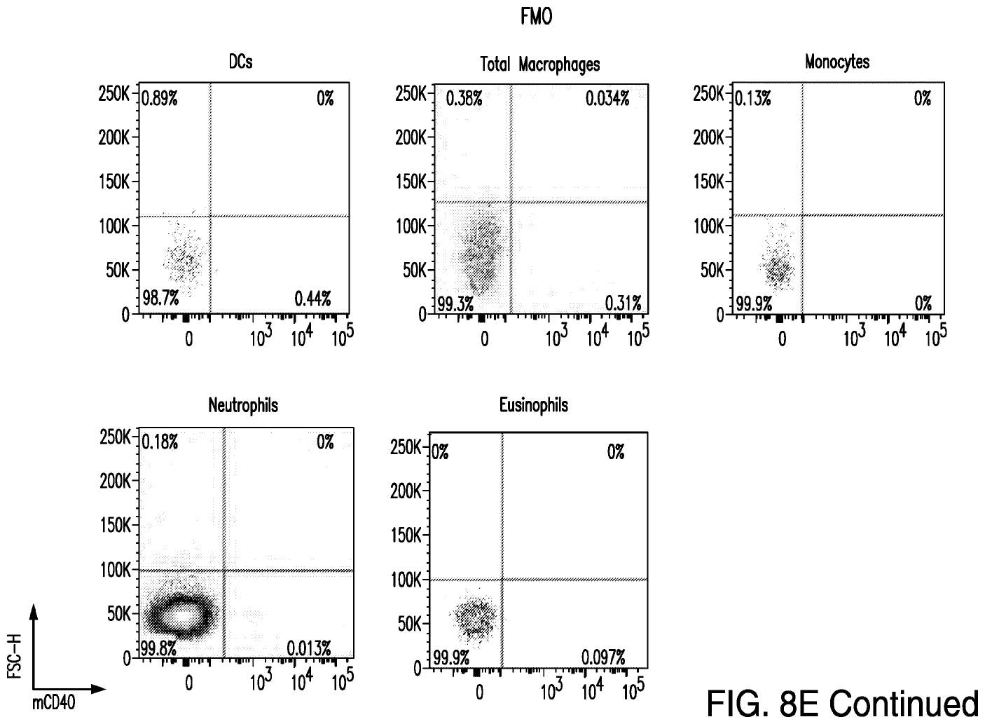

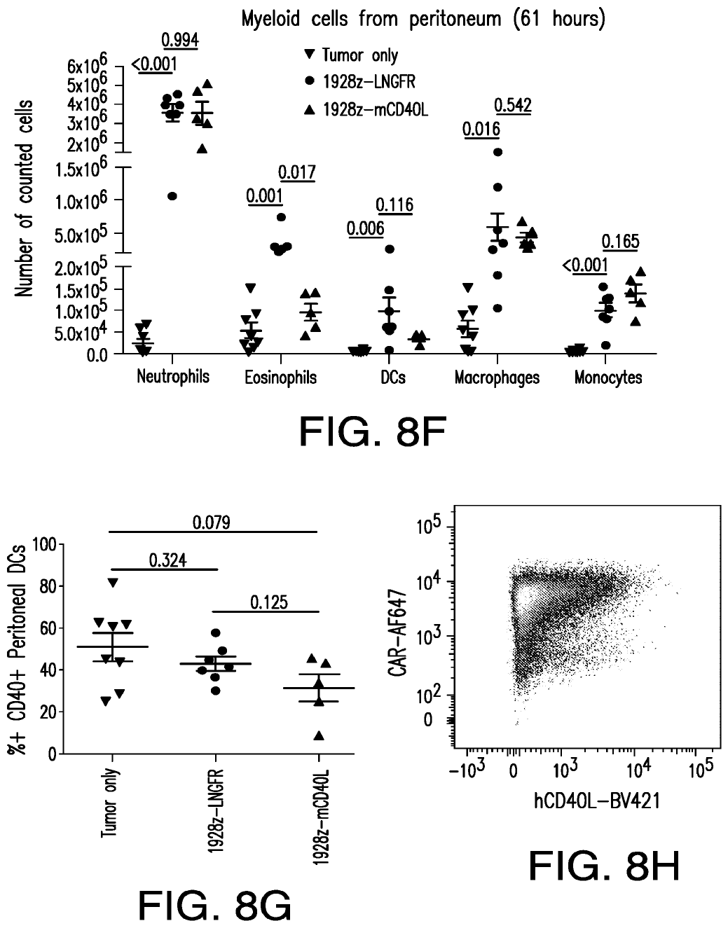

The xenogeneic model was again taken advantage of to further probe the role of T cell-myeloid cell interactions by expressing murine CD40L in human CAR T cells. CD40L is mainly expressed by T cells, while DCs, monocytes and macrophages express the CD40 receptor¹⁶, but human CD40L does not functionally interact with the murine CD40 receptor¹⁷. mCD40L was constitutively expressed in CAR T cells using a bicistronic vector (FIGS. 3A and 8 A). CD40L expression resulted in more severe and sustained weight loss in mice (FIGS. 3B and 3M) and significantly increased mortality in the 1928z-mCD40L group (FIG. 8B). Moreover, comparable numbers of recruited myeloid cells in both CAR and CAR/mCD40L treatment groups (FIGS. 8C and 8F) suggested that the increased severity of CRS was due to qualitative and not quantitative changes in the myeloid compartment. Indeed, in mice receiving 1928z-mCD40L an overwhelming accumulation of activated macrophages was noticed (FIGS. 3C and 3D). Notably, while cell-surface expression of CD40 was exclusive to macrophages and DCs in peritoneal myeloid cells (FIG. 8E), only macrophages down-regulated its expression in the presence of CAR/mCD40L T cells (FIGS. 3G, 8 C, 8D, 8F and 8G). Down-regulation of cell-surface CD40 was in accordance with functional CD40 signaling¹⁸⁻²⁰, thus establishing that macrophages were directly affected by the introduction of mCD40L. In line with the increased severity of the observed CRS, levels of murine inflammatory cytokines were also significantly increased, including IL-6, which was known to be directly induced by CD40L signaling²¹ (FIGS. 3F-3 I and 3 P). These findings further supported the hypothesis that proximal interactions of CAR T cells and myeloid cells were critical to the severity of CRS.

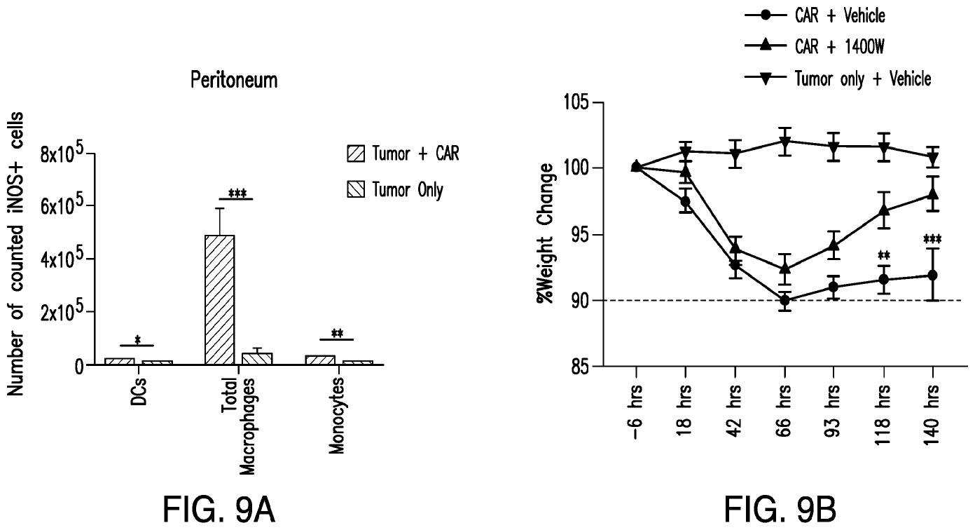

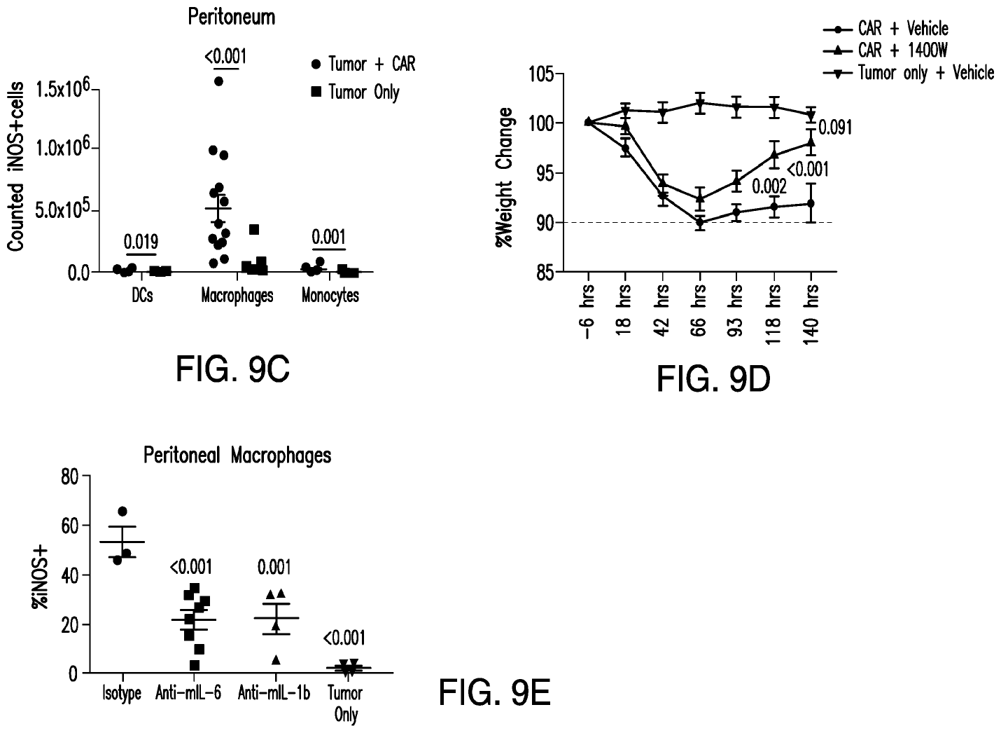

To further investigate the function of macrophages, they were examined for expression of inducible Nitric Oxide Synthase (iNOS), an enzyme known to be predominantly expressed by activated macrophages²². In line with the finding that local interactions with CAR T cells were a key driver of CRS, only peritoneal but not splenic or bone marrow myeloid populations significantly increased iNOS production (FIGS. 3J and 3Q). Macrophages showed the highest induction of iNOS (FIGS. 3J and 3Q) and numerically were the most significant population expressing the protein (FIGS. 9A and 9C). While well-regulated iNOS activity could have protective effects, aberrant Nitric Oxide (NO) production could lead to adverse events such as severe vasodilation and subsequent hypotension^{23, 24}, a clinical entity often observed in CAR T cells trials as pressor-resistant hypotension⁴. To test the relevance of iNOS in this mouse model, mice were treated with either of two selective iNOS inhibitors, L-NIL²⁵, or 1400W²⁶. L-NIL-treated mice exhibited a robust reversal of toxicity as witnessed by weight loss (FIGS. 3K and 3R) in a non-lethal CRS episode (data not shown). Treatment with 1400W significantly prevented mortality prevention from CRS and reversed toxicity (FIGS. 3L, 3S and 9B). Taken together, these data support that modulation of macrophage activity radically alters CRS outcomes.

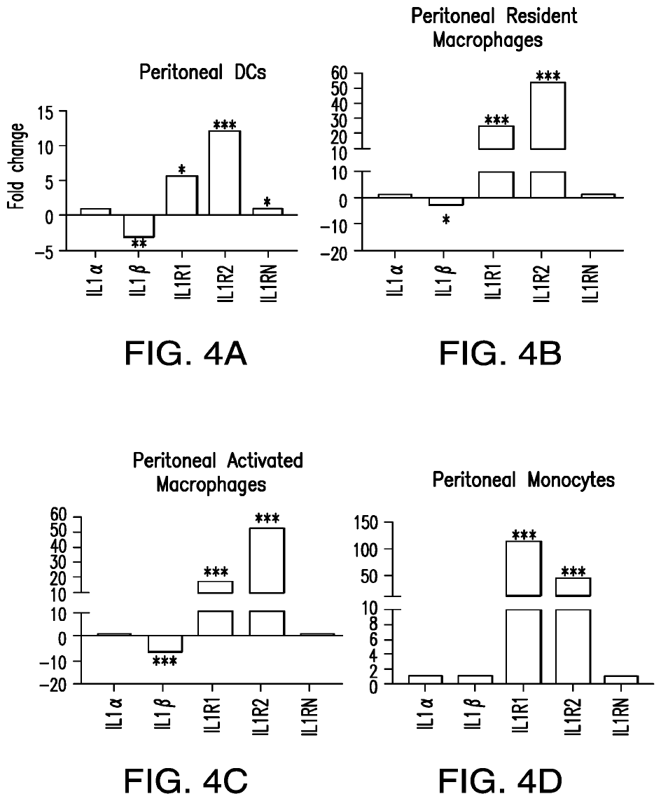

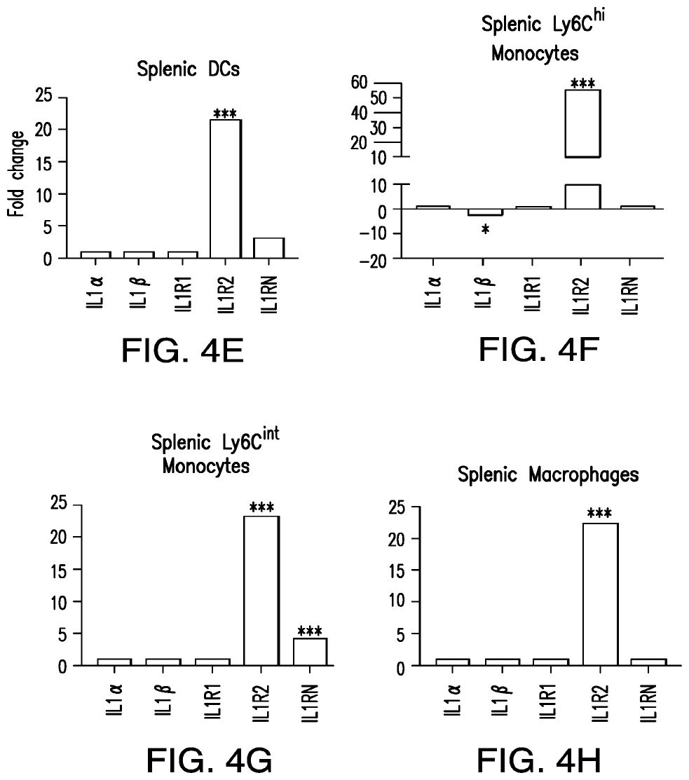

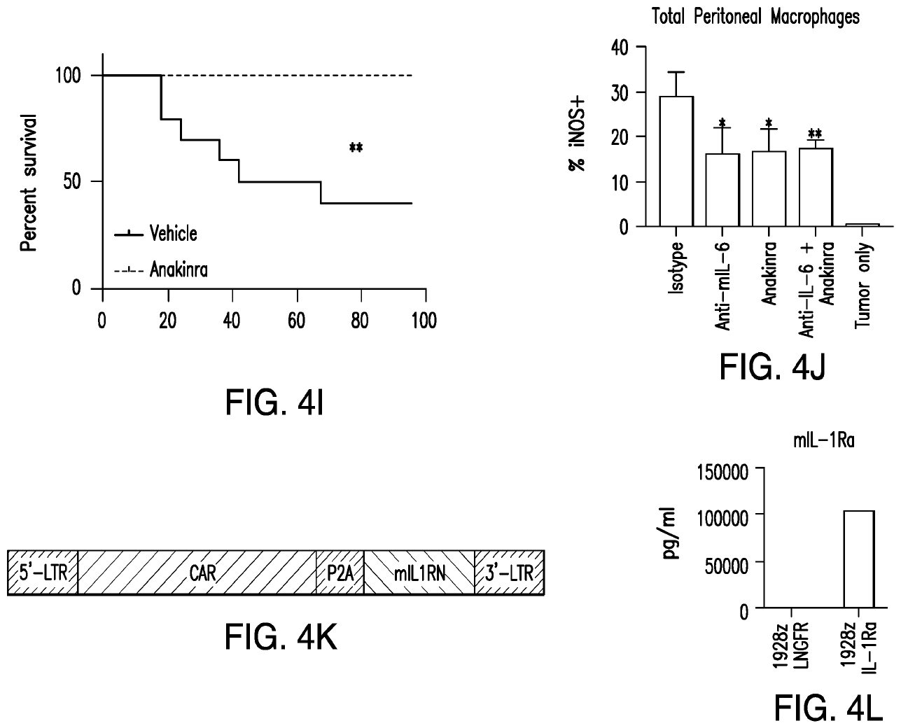

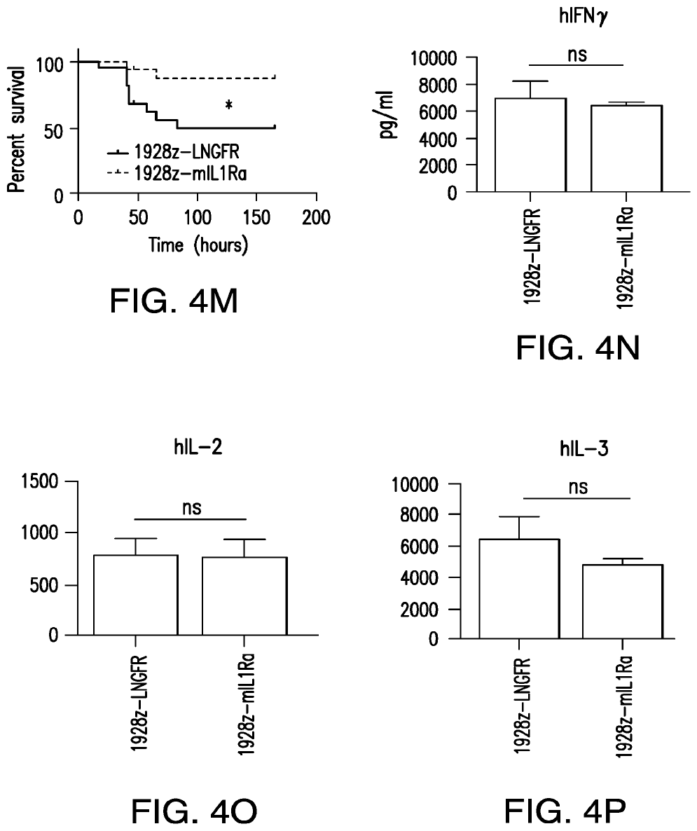

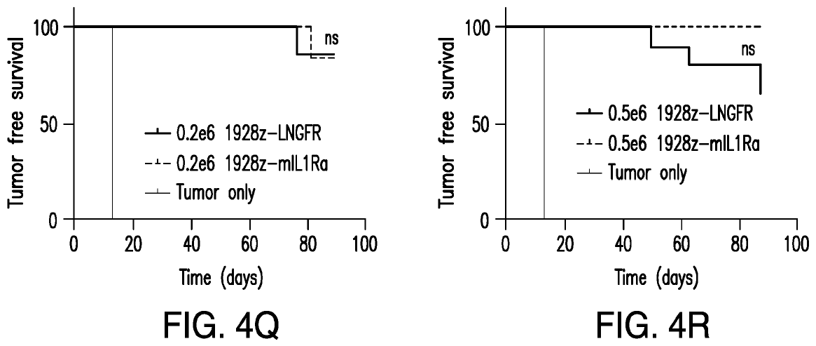

Having observed the importance of iNOS in this model, the role of IL-6 and IL-1 were further examined as both cytokines were known inducers of iNOS^{27, 28}. The RNAseq data in myeloid cell types harvested at the onset of CRS showed that the type 1 IL1 receptor (IL-1R1), which is required for functional IL-1 signaling, was exclusively upregulated in peritoneal myeloid cells but not splenic cells (FIGS. 4A-4D). Conversely, splenic myeloid cells upregulated the type 2 IL-1 receptor (IL-1R2), which does not functionally signal and serves as a decoy receptor. Moreover, the upregulation of IL-1 receptor antagonist (IL1RN/IL-1Ra) was observed in splenic myeloid cells (FIGS. 4E-4H), which suggested a natural response towards IL-1 signaling inhibition derived from the spleen in contrast to a mixed response in the peritoneum²⁹. In light of these findings it was hypothesized that native IL-1 suppression was insufficient to inhibit pro-inflammatory effects of IL-1 and intervening pharmacologically to enhance anti-IL-1 responses would mitigate CRS symptoms. Indeed, IL-1 blockade by Anakinra completely abrogated CRS-related mortality (FIG. 4I). In order to obtain more insight in the protective mechanism of IL-1 blocking and how it compared to IL-6 blocking, the impact of Anakinra on macrophage activation was assessed through induction of iNOS expression levels. Interestingly, both blockades resulted in similarly reduced iNOS⁺ macrophage fractions. Combinatorial IL-1/IL-6 blockade, however, did not further decrease the fraction of iNOS+ macrophages, suggesting that the inhibition afforded by these blockades affects the same pathway (FIGS. 4J and 9E). Therefore, downregulation of iNOS was identified as a unifying mechanism by which IL-6 and IL-1 blockades can in part protect mice from ongoing, acute CRS.

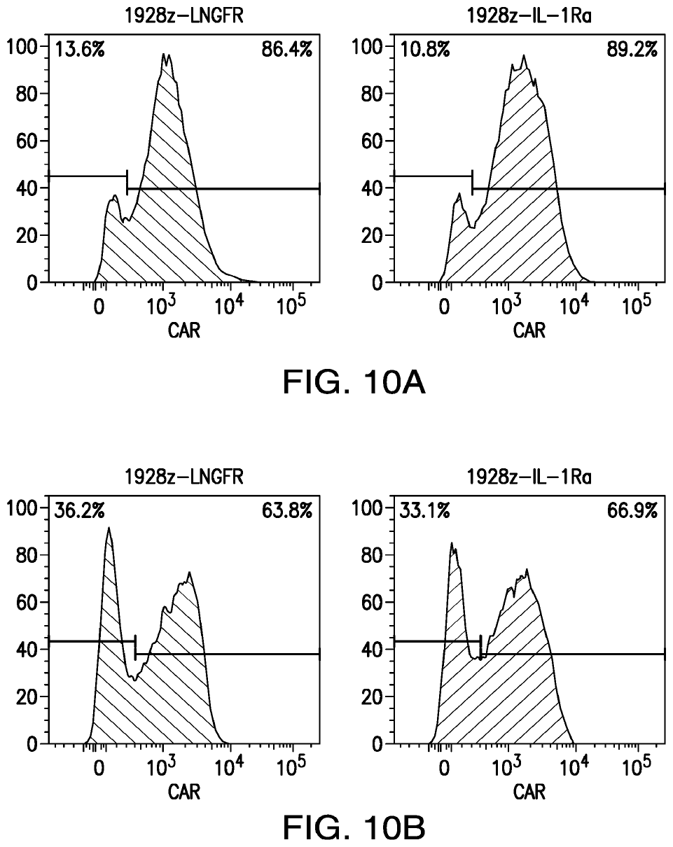

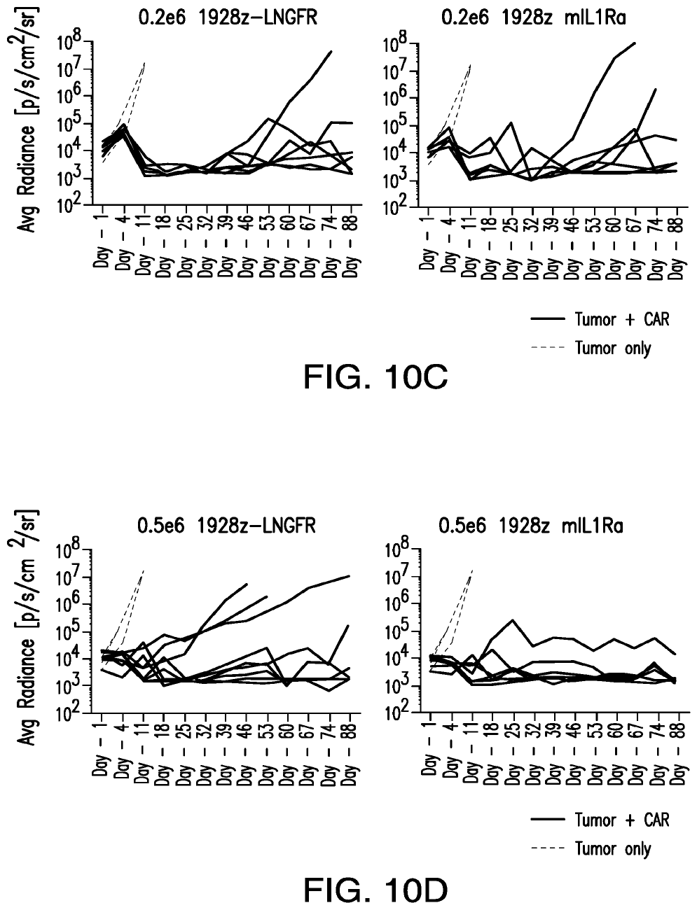

In order to prevent CRS mortality without any exogenous intervention, the endogenous IL-1 inhibitor, IL-1 receptor antagonist (IL1RN/IL-1Ra) was taken advantage of and a novel CAR construct was designed, which constitutively produces IL-1Ra (FIGS. 4K and 4L). First, it was confirmed that this novel construct protected from CRS-associated mortality (FIGS. 4M and 10A) while CAR-T cell activation remained unaffected as assessed by CAR T cell-derived serum cytokine levels (FIGS. 4N-4 P). Second, the “stress test” model was employed to ascertain whether long-term antitumor efficacy³⁰ at limiting CAR T cell doses could be affected by IL-1Ra expression. At two different doses, 1928z-mIL-1Ra matched the anti-tumor efficacy of 1928z-LNGFR (FIGS. 4Q and 4R and 10B-10D). Therefore, a novel actionable target for CRS was identified and a “CRS-blocking” CAR construct was design that largely prevents CRS-associated mortality in mice without any further external intervention. The benefits of an IL-1 blockade through IL-1Ra are especially intriguing given its ability to cross the blood brain barrier³¹, whereas tocilizumab presumably cannot⁴. Human microglia are known to be activated by IL-1 to produce iNOS and pro-inflammatory cytokines^{32, 33} and therefore blocking IL-1 could both protect from severe CRS and reduce the severity of CART cell related neurotoxicity.

TABLE 2

| | Elevated in CRS Patients | | |
|---|---|---|---|
| | (Davila et al. (20014), | | |
| | Teachey et al. (2016), | Elevated in | Source in |
| Cytokine | Hay et el. (2017) | mouse model | mouse model |
| IFNg | Yes | Yes | Human |
| TNFa | Yes | Yes | Human |
| GM-CSF | Yes | Yes | Human |
| G-CSF | Yes | Yes | Mouse |
| IL-1b | No | No | Mouse**** |
| IL-2 | No | Yes | Human |
| IL-5 | Yes/No/Yes*** | Yes*** | Mouse |
| IL-6 | Yes | Yes | Mouse |
| IL-8** | Yes | Yes | Mouse |
| IL-10 | Yes | Yes* | Human |
| IL-12 | No | No | Mouse |
| IL-13 | No | No | Human |
| IL-15 | No | No | Mouse |
| IL-17 | No | No | Mouse |
| CCL2 | Yes | Yes | Mouse |
| CCL3 | Yes | Yes | Both |
| CCL4 | Yes | Yes | Mouse |
| CCL5 | No | No | Mouse |
| TNFRII | Yes | Yes* | Human |
| Eotaxin | No | No | Not detected |

*Only with CD40L
**CXCL1 is the murine equivalent of human IL-8
***Elevated in Davila et al., Hay et al.
****When above detection levels

Table 2 shows cytokines differentially upregulated in patients with CRS or severe CRS in the available literature (Davila et al. 2014, Teachey et al. 2016, Hay et al. 2017) compared to cytokines upregulated in mice with CRS or severe CRS. Mouse cytokine data were compiled from multiple independent experiments. Green boxes indicate clinical agreement with our mouse model, red boxes indicate differences in this mouse model and orange indicates differing clinical observations between the three clinical studies. Main source of cytokine is noted under “source in mouse model” column. When a cytokine was produced by both murine and human cells the main source was determined by comparing the fold difference between the averages of the two sources. When the fold-difference was less than four-fold the source was attributed as “both”.

TABLE 3

| Cytokine | |
|---|---|
| | Human on mouse |
| IFNg | No³⁸ |
| TNFa | Partial⁴⁰ |
| GM-CSF | No³⁹ |
| G-CSF | Yes³⁹ |
| IL-2 | Yes³⁹ |
| IL-3 | No³⁹ |
| IL-8 | Yes⁴⁰ |
| IL-10 | Yes⁴⁰ |
| IL-13 | Yes⁴⁰ |
| IL-17 | Yes⁴⁰ |
| | Mouse on human |
| G-CSF | Yes³⁹ |
| IL-5 | Yes⁴⁰ |
| IL-6 | No³⁹ |
| IL-15 | Yes³⁹ |

Table 3 is a cross-species reactivity chart of human and murine cytokines detected in our mouse model. “Human on mouse” column indicates whether cytokines of human origin are active on the cognate murine receptor. “Mouse on human” column indicates whether cytokines of murine origin are active on the cognate human receptor. Human TNF-α can signal through the murine p55 TNF receptor but not the p75 TNF receptor.

In summary, the myeloid system was directly involved in the pathogenesis of CRS. It was established that CAR-T cells activate and recruit myeloid cells within the microenvironment of antitumor activity. An important concept framed by these findings is the impact of co-localization of CAR T cells with myeloid cells within the milieu of antitumor activity. Selectively modulating macrophage activity with either CD40L, iNOS inhibitors or Anakinra revealed their integral role in defining CRS outcomes. IL-1 was further identified as a novel actionable target, suitable to treat acute CRS and diminish its severity. These findings informed the design of an IL-1Ra-secreting CAR construct that can demonstrably prevent CRS-related mortality while maintaining intact antitumor efficacy.

### REFERENCES

1. Sadelain, M., Riviere, I. & Riddell, S. Therapeutic T cell engineering. Nature 545, 423-431 (2017). 28541315
2. Maude, S. L., Frey, N., Shaw, P. A., Aplenc, R., Barrett, D. M., Bunin, N. J., Chew, A., Gonzalez, V. E., Zheng, Z., Lacey, S. F., Mahnke, Y. D., Melenhorst, J. J., Rheingold, S. R., Shen, A., Teachey, D. T., Levine, B. L., June, C. H., Porter, D. L. & Grupp, S. A. Chimeric antigen receptor T cells for sustained remissions in leukemia. N Engl J Med 371, 1507-1517 (2014). 25317870
3. Davila, M. L., Riviere, I., Wang, X., Bartido, S., Park, J., Curran, K., Chung, S. S., Stefanski, J., Borquez-Ojeda, O., Olszewska, M., Qu, J., Wasielewska, T., He, Q., Fink, M., Shinglot, H., Youssif, M., Satter, M., Wang, Y., Hosey, J., Quintanilla, H., Halton, E., Bernal, Y., Bouhassira, D. C., Arcila, M. E., Gonen, M., Roboz, G. J., Maslak, P., Douer, D., Frattini, M. G., Giralt, S., Sadelain, M. & Brentjens, R. Efficacy and toxicity management of 19-28z CAR T cell therapy in B cell acute lymphoblastic leukemia. Science translational medicine 6, 224ra225 (2014). 24553386
4. Neelapu, S. S., Tummala, S., Kebriaei, P., Wierda, W., Gutierrez, C., Locke, F. L., Komanduri, K. V., Lin, Y., Jain, N., Daver, N., Westin, J., Gulbis, A. M., Loghin, M. E., de Groot, J. F., Adkins, S., Davis, S. E., Rezvani, K., Hwu, P. & Shpall, E. J. Chimeric antigen receptor T-cell therapy—assessment and management of toxicities. Nat Rev Clin Oncol (2017). 28925994
5. Lee, D. W., Kochenderfer, J. N., Stetler-Stevenson, M., Cui, Y. K., Delbrook, C., Feldman, S. A., Fry, T. J., Orentas, R., Sabatino, M., Shah, N. N., Steinberg, S. M., Stroncek, D., Tschernia, N., Yuan, C., Zhang, H., Zhang, L., Rosenberg, S. A., Wayne, A. S. & Mackall, C. L. T cells expressing CD19 chimeric antigen receptors for acute lymphoblastic leukaemia in children and young adults: a phase 1 dose-escalation trial. Lancet 385, 517-528 (2015). 25319501
6. Oluwole, O. O. & Davila, M. L. At The Bedside: Clinical review of chimeric antigen receptor (CAR) T cell therapy for B cell malignancies. Journal of leukocyte biology 100, 1265-1272 (2016). 27354412
7. Brudno, J. N. & Kochenderfer, J. N. Toxicities of chimeric antigen receptor T cells: recognition and management. Blood 127, 3321-3330 (2016). 27207799
8. Bonifant, C. L., Jackson, H. J., Brentjens, R. J. & Curran, K. J. Toxicity and management in CAR T-cell therapy. Mol Ther Oncolytics 3, 16011 (2016). 27626062
9. Barrett, D. M., Teachey, D. T. & Grupp, S. A. Toxicity management for patients receiving novel T-cell engaging therapies. Curr Opin Pediatr 26, 43-49 (2014). 24362408
10. Cray, C., Zaias, J. & Altman, N. H. Acute phase response in animals: a review. Comp Med 59, 517-526 (2009). 20034426
11. Meek, R. L., Eriksen, N. & Benditt, E. P. Murine serum amyloid A3 is a high density apolipoprotein and is secreted by macrophages. Proc Natl Acad Sci USA 89, 7949-7952 (1992). 1518819
12. van der Stegen, S. J., Davies, D. M., Wilkie, S., Foster, J., Sosabowski, J. K., Burnet, J., Whilding, L. M., Petrovic, R. M., Ghaem-Maghami, S., Mather, S., Jeannon, J. P., Parente-Pereira, A. C. & Maher, J. Preclinical in vivo modeling of cytokine release syndrome induced by ErbB-retargeted human T cells: identifying a window of therapeutic opportunity? Journal of immunology 191, 4589-4598 (2013). 24062490
13. Raziuddin, A., Sarkar, F. H., Dutkowski, R., Shulman, L., Ruddle, F. H. & Gupta, S. L. Receptors for human alpha and beta interferon but not for gamma interferon are specified by human chromosome 21. Proc Natl Acad Sci USA 81, 5504-5508 (1984). 6206498
14. Manz, M. G. Human-hemato-lymphoid-system mice: opportunities and challenges. Immunity 26, 537-541 (2007). 17521579
15. Rincon, M. Interleukin-6: from an inflammatory marker to a target for inflammatory diseases. Trends in immunology 33, 571-577 (2012). 22883707
16. Quezada, S. A., Jarvinen, L. Z., Lind, E. F. & Noelle, R. J. CD40/CD154 interactions at the interface of tolerance and immunity. Annual review of immunology 22, 307-328 (2004). 15032580
17. Bossen, C., Ingold, K., Tardivel, A., Bodmer, J. L., Gaide, O., Hertig, S., Ambrose, C., Tschopp, J. & Schneider, P. Interactions of tumor necrosis factor (TNF) and TNF receptor family members in the mouse and human. The Journal of biological chemistry 281, 13964-13971 (2006). 16547002
18. Tucker, T. A. & Schwiebert, L. M. CD40 ligation decreases its protein half-life at the cell surface. European journal of immunology 38, 864-869 (2008). 18253927
19. Wang, H., Zhao, C., Zhang, M., Lee, C. M., Reddy, E. P. & Kung, S. K. A novel role of the scaffolding protein JLP in tuning CD40-induced activation of dendritic cells. Immunobiology 218, 835-843 (2013). 23182713
20. Wang, H. M., Yan, Q., Yang, T., Cheng, H., Du, J., Yoshioka, K., Kung, S. K. & Ding, G. H. Scaffold protein JLP is critical for CD40 signaling in B lymphocytes. The Journal of biological chemistry 290, 5256-5266 (2015). 25586186
21. Elgueta, R., Benson, M. J., de Vries, V. C., Wasiuk, A., Guo, Y. & Noelle, R. J. Molecular mechanism and function of CD40/CD40L engagement in the immune system. Immunol Rev 229, 152-172 (2009). 19426221
22. Murray, P. J. & Wynn, T. A. Protective and pathogenic functions of macrophage subsets. Nat Rev Immunol 11, 723-737 (2011). 21997792
23. Ohashi, Y., Kawashima, S., Hirata, K., Yamashita, T., Ishida, T., Inoue, N., Sakoda, T., Kurihara, H., Yazaki, Y. & Yokoyama, M. Hypotension and reduced nitric oxide-elicited vasorelaxation in transgenic mice overexpressing endothelial nitric oxide synthase. The Journal of clinical investigation 102, 2061-2071 (1998). 9854041
24. Titheradge, M. A. Nitric oxide in septic shock. Biochim Biophys Acta 1411, 437-455 (1999). 10320674
25. Moore, W. M., Webber, R. K., Jerome, G. M., Tjoeng, F. S., Misko, T. P. & Currie, M. G. L-N6-(1-iminoethyl)lysine: a selective inhibitor of inducible nitric oxide synthase. J Med Chem 37, 3886-3888 (1994). 7525961
26. Garvey, E. P., Oplinger, J. A., Furfine, E. S., Kiff, R. J., Laszlo, F., Whittle, B. J. & Knowles, R. G. 1400W is a slow, tight binding, and highly selective inhibitor of inducible nitric-oxide synthase in vitro and in vivo. The Journal of biological chemistry 272, 4959-4963 (1997). 9030556
27. Dinarello, C. A. Immunological and inflammatory functions of the interleukin-1 family. Annual review of immunology 27, 519-550 (2009). 19302047
28. Saini, A. S., Shenoy, G. N., Rath, S., Bal, V. & George, A. Inducible nitric oxide synthase is a major intermediate in signaling pathways for the survival of plasma cells. Nature immunology 15, 275-282 (2014). 24441790
29. Sims, J. E. & Smith, D. E. The IL-1 family: regulators of immunity. Nat Rev Immunol 10, 89-102 (2010). 20081871
30. Zhao, Z., Condomines, M., van der Stegen, S. J. C., Perna, F., Kloss, C. C., Gunset, G., Plotkin, J. & Sadelain, M. Structural Design of Engineered Costimulation Determines Tumor Rejection Kinetics and Persistence of CART Cells. Cancer Cell 28, 415-428 (2015). 26461090
31. Gutierrez, E. G., Banks, W. A. & Kastin, A. J. Blood-borne interleukin-1 receptor antagonist crosses the blood-brain barrier. Journal of neuroimmunology 55, 153-160 (1994). 7829665
32. Liu, J., Zhao, M. L., Brosnan, C. F. & Lee, S. C. Expression of type II nitric oxide synthase in primary human astrocytes and microglia: role of IL-1beta and IL-1 receptor antagonist. Journal of immunology 157, 3569-3576 (1996). 8871657
33. Tarassishin, L., Suh, H. S. & Lee, S. C. LPS and IL-1 differentially activate mouse and human astrocytes: role of CD14. Glia 62, 999-1013 (2014). 24659539
34. Gade, T. P., Hassen, W., Santos, E., Gunset, G., Saudemont, A., Gong, M. C., Brentjens, R., Zhong, X. S., Stephan, M., Stefanski, J., Lyddane, C., Osborne, J. R., Buchanan, I. M., Hall, S. J., Heston, W. D., Riviere, I., Larson, S. M., Koutcher, J. A. & Sadelain, M. Targeted elimination of prostate cancer by genetically directed human T lymphocytes. Cancer Res 65, 9080-9088 (2005). 16204083
35. Maher, J., Brentjens, R. J., Gunset, G., Riviere, I. & Sadelain, M. Human T-lymphocyte cytotoxicity and proliferation directed by a single chimeric TCRzeta/CD28 receptor. Nat Biotechnol 20, 70-75 (2002). 11753365
36. Riviere, I., Brose, K. & Mulligan, R. C. Effects of retroviral vector design on expression of human adenosine deaminase in murine bone marrow transplant recipients engrafted with genetically modified cells. Proc Natl Acad Sci USA 92, 6733-6737 (1995). 7624312
37. M. C., Latouche, J. B., Krause, A., Heston, W. D., Bander, N. H. & Sadelain, M. Cancer patient T cells genetically targeted to prostate-specific membrane antigen specifically lyse prostate cancer cells and release cytokines in response to prostate-specific membrane antigen. Neoplasia 1, 123-127 (1999). 10933046.
38. Raziuddin, A., et al. Receptors for human alpha and beta interferon but not gamma interferon are specified by human chromosome 21. Proc Natl Acad Sci USA 81, 5504-5508 (1984).
39. Manz, M. G. Human-hemato-lymphoid-system mice: opportunities and challenges. Immunity 26, 537-541 (2007).
40. Fitzgerald, K., O'Neill, L., Gearing, A. & Callard, R. The Cytokine Factsbook, (Academic Press/Elsevier, 2001).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising:
(a) an antigen-recognizing receptor that binds to an antigen, and
(b) an exogenous IL-1Ra polypeptide.

2. An immunoresponsive cell comprising:
(a) an antigen-recognizing receptor that binds to an antigen, and
(b) a modified promoter at an endogenous IL-1Ra gene locus.

3. The immunoresponsive cell of claim 2, wherein the modified promoter enhances gene expression of the endogenous IL-1Ra gene.

4. The immunoresponsive cell of claim 2, wherein the modification comprises replacement of an endogenous promoter with a constitutive promoter or an inducible promoter, or insertion of a constitutive promoter or inducible promoter to the promoter region of the endogenous IL-1Ra gene locus.

5. The immunoresponsive cell of claim 4, wherein the constitutive promoter is selected from the group consisting of a CMV promoter, an EF1a promoter, a SV40 promoter, a PGK1 promoter, a Ubc promoter, a beta-actin promoter, and a CAG promoter, and/or the inducible promoter is selected from the group consisting of a tetracycline response element (TRE) promoter and an estrogen response element (ERE) promoter.

6. The immunoresponsive cell of claim 1, wherein the antigen is a tumor antigen or a pathogen antigen, optionally wherein the antigen is a tumor antigen.

7. The immunoresponsive cell of claim 1, wherein the exogenous IL-1Ra polypeptide is secreted.

8. The immunoresponsive cell of claim 1, wherein said antigen-recognizing receptor is a T cell receptor (TCR) or a chimeric antigen receptor (CAR).

9. The immunoresponsive cell of claim 1, wherein the antigen-recognizing receptor and/or the exogenous IL-1Ra polypeptide is expressed from a vector.

10. The immunoresponsive cell of claim 1, wherein the cell is selected from the group consisting of a T cell, a Natural Killer (NK) cell, a pluripotent stem cell from which lymphoid cells may be differentiated, a macrophage, a neutrophil, a monocyte, and a dendritic cell.

11. The immunoresponsive cell of claim 1, wherein the cell is a T cell.

12. The immunoresponsive cell of claim 11, wherein the T cell is selected from the group consisting of a cytotoxic T lymphocyte (CTL), a regulatory T cell, a Natural Killer T (NKT) cell, and combinations thereof.

13. The immunoresponsive cell of claim 1, wherein said immunoresponsive cell is autologous or allogeneic.

14. The immunoresponsive cell of claim 6, wherein the tumor antigen is selected from the group consisting of CD19, MUC16, MUC1, CAIX, CEA, CD8, CD7, CD10, CD20, CD22, CD30, CD33, CLL1, CD34, CD38, CD41, CD44, CD49f, CD56, CD74, CD133, CD138, a cytomegalovirus (CMV) infected cell antigen, EGP-2, EGP-40, EpCAM, Erb-B2, Erb-B3, Erb-B4, FBP, Fetal acetylcholine receptor, folate receptor-α, GD2, GD3, HER-2, hTERT, IL-13R-a2, κ-light chain, KDR, LeY, L1 cell adhesion molecule, MAGE-A1, Mesothelin, ERBB2, MAGEA3, p53, MART1, GP100, Proteinase3 (PR1), Tyrosinase, Survivin, hTERT, EphA2, NKG2D ligands, NY-ESO-1, oncofetal antigen (h5T4), PSCA, PSMA, ROR1, TAG-72, VEGF-R2, WT-1, BCMA, CD123, CD44V6, NKCS1, EGF1R, EGFR-VIII, ERBB, ITGB5, PTPRJ, SLC30A1, EMC10, SLC6A6, TNFRSF1B, CD82, ITGAX, CR1, DAGLB, SEMA4A, TLR2, LTB4R, P2RY13, LILRB2, EMB, CD96, LILRB3, LILRA6, LILRA2, ADGRE2, LILRB4, CD70, CCR1, CCR4, TACI, TRBC1, and TRBC2.

15. The immunoresponsive cell of claim 14, wherein said antigen is CD19.

16. The immunoresponsive cell of claim 1, wherein said IL-1Ra polypeptide comprises a heterologous signal sequence at the amino-terminus.

17. The immunoresponsive cell of claim 16, wherein said heterologous signal sequence is an IL-2 signal sequence.

18. The immunoresponsive cell of claim 8, wherein the antigen-recognizing receptor is a CAR.

19. The immunoresponsive cell of claim 18, wherein the CAR comprises an extracellular antigen-binding domain, a transmembrane domain, and an intracellular signaling domain.

20. The immunoresponsive cell of claim 1, wherein the IL-1Ra peptide comprises (a) an amino acid sequence that is at least about 80% homologous or identical to the sequence set forth in SEQ ID NO: 4 or SEQ ID NO: 21; or (b) the amino acid sequence set forth in SEQ ID NO: 4 or SEQ ID NO: 21.

21. An immunoresponsive cell comprising a modified CD40L.

22. The immunoresponsive cell of claim 21, wherein the modification is selected from the group consisting of knock-down of CD40L, knock-out of CD40L, introduction of one or more mutation in a CD40L gene, modification of the endogenous promoter of a CD40L gene, modification of the endogenous enhancer elements of a CD40L gene, modification of the transcription factors that control CD40L expression, and combinations thereof.

23. A pharmaceutical composition comprising an effective amount of an immunoresponsive cell of claim 1 and a pharmaceutically acceptable excipient.

24. A pharmaceutical composition comprising an effective amount of an immunoresponsive cell of claim 21 and a pharmaceutically acceptable excipient.

25. A method of reducing tumor burden in a subject, and/or treating and/or preventing a neoplasm, and/or lengthening survival of a subject having a neoplasm, and/or reducing at least one symptom of cytokine release syndrome (CRS) in a subject, and/or reducing the level of a chemokine in a subject, the method comprising administering to the subject an immunoresponsive cell of claim 1 or a pharmaceutical composition comprising thereof.

26. A method of reducing tumor burden in a subject, and/or treating and/or preventing a neoplasm, and/or lengthening survival of a subject having a neoplasm, and/or reducing at least one symptom of cytokine release syndrome (CRS) in a subject, and/or reducing the level of a chemokine in a subject, the method comprising administering to the subject an immunoresponsive cell of claim 21 or a pharmaceutical composition comprising thereof.

27. A method of reducing tumor burden in a subject, and/or treating and/or preventing a neoplasm, and/or lengthening survival of a subject having a neoplasm, and/or reducing at least one symptom of cytokine release syndrome (CRS) in a subject, and/or reducing the level of a chemokine in a subject, the method comprising administering to the subject (i) an antibody that binds to CD40L and (ii) an immunoresponsive cell comprising an antigen-recognizing receptor that binds to an antigen.

28. A method of reducing tumor burden in a subject, and/or treating and/or preventing a neoplasm, and/or lengthening survival of a subject having a neoplasm, and/or reducing at least one symptom of cytokine release syndrome (CRS) in a subject, and/or reducing the level of a chemokine in a subject, the method comprising administering to the subject (i) an inhibitor of IL-1 signaling and (ii) an immunoresponsive cell comprising an antigen-recognizing receptor that binds to an antigen.

29. A method for producing an antigen-specific immunoresponsive cell of claim 1, the method comprising introducing into an immunoresponsive cell (a) a first nucleic acid sequence encoding the antigen-recognizing receptor; and (b) a second nucleic sequence encoding the exogenous IL-1Ra polypeptide, wherein each of the first and second nucleic acid sequence optionally operably linked to a promoter element.

30. A method for producing an antigen-specific immunoresponsive cell of claim 21, the method comprising introducing into an immunoresponsive cell (a) a first nucleic acid sequence encoding the antigen-recognizing receptor that binds to an antigen; and (b) a second nucleic sequence encoding the modified CD40L, wherein each of the first and second nucleic acid sequence optionally operably linked to a promoter element.

31. A nucleic acid composition comprising (a) a first nucleic acid sequence encoding an antigen-recognizing receptor and (b) a second nucleic acid sequence encoding an exogenous IL-1Ra polypeptide, each optionally operably linked to a promoter element.

32. A nucleic acid composition comprising (a) a first nucleic acid sequence encoding an antigen-recognizing receptor and (b) a second nucleic acid sequence encoding a modified CD40L, each optionally operably linked to a promoter element.

33. A vector comprising the nucleic acid composition of claim 31.

34. A vector comprising the nucleic acid composition of claim 32.

35. The vector of claim 33, wherein the vector is a retroviral vector.

36. The vector of claim 34, wherein the vector is a retroviral vector.

37. A kit comprising an immunoresponsive cell of claim 1.

38. A kit comprising an immunoresponsive cell of claim 21.

39. A mouse exhibiting one or more cytokine release syndrome (CRS)-related symptom, the mouse comprising:
(a) a tumor cell;
(b) an immunoresponsive cell comprising an antigen-recognizing receptor that binds to an antigen, wherein the immunoresponsive cell is present in an amount that is sufficient to induce one or more CRS-related symptom in the mouse.

40. The mouse of claim 39, wherein
(a) the mouse is an immunocompetent mouse or an immunodeficient mouse; and/or
(b) the tumor cell is a human tumor cell or a murine tumor cell; and/or the immunoresponsive cell is a T cell; and/or
(c) the antigen-recognizing receptor comprised in the immunoresponsive cell is a CAR; and/or
(d) the one or more CRS-related symptom is selected from the group consisting of elevated level of one or more pro-inflammatory cytokine, rapid weight loss, piloerection, reduced activity, general presentation of malaise, mortality and any combination thereof.

41. A method of screening an agent that is capable of preventing, alleviating and/or treating cytokine release syndrome (CRS), comprising (a) administering a test agent to the mouse of claim 39, and (b) measuring one or more CRS-related symptom in the mouse; and wherein alleviation of one or more CRS-related symptoms is indicates that the test agent is likely to be capable of preventing, alleviating and/or treating CRS.
